(19) United States
(12) Patent Application Publication
(10) Pub. No.: US 2025/0207202 A1
(43) Pub. Date: Jun. 26, 2025

(54) METHODS AND COMPOSITIONS FOR IDENTIFYING HOX GENE SIGNATURES TO ASSIGN SPECIFIC AND EFFECTIVE THERAPIES IN ACUTE MYELOID LEUKEMIA AND OTHER CANCERS

(71) Applicant: Ohio State Innovation Foundation, Columbus, OH (US)

(72) Inventors: Christopher OAKES, Columbus, OH (US); James BLACHLY, Columbus, OH (US); John BYRD, Cincinnati, OH (US)

(21) Appl. No.: 18/876,856

(22) PCT Filed: Jul. 3, 2023
(86) PCT No.: PCT/US2023/026829

§ 371 Date: Dec. 19, 2024

Related U.S. Application Data
(60) Provisional application No. 63/357,772, filed on Jul. 1, 2022.

(51) Int. Cl. C12Q 1/6886; C12Q 1/6806; C12Q 1/6809; C12Q 1/6827

(52) U.S. Cl. CPC C12Q 1/6886; C12Q 1/6806; C12Q 1/6809; C12Q 1/6827; C12Q 2600/106; C12Q 2600/154

(57) ABSTRACT

The present disclosure provides kits and/or methods of detecting and identifying epigenetic patterns associated with acute myeloid leukemia and other cancers. The present disclosure also relates to treating, preventing, ameliorating, or reducing acute myeloid leukemia and other cancers.

### CROSS-REFERENCE TO RELATED APPLICATIONS

This application claims priority to, and the benefit of, U.S. Provisional Patent Application No. 63/357,772, filed Jul. 1, 2022, which is incorporated by reference herein in its entirety.

### FIELD

The present disclosure provides kits and/or methods of detecting and identifying epigenetic patterns associated with acute myeloid leukemia and other cancers. The present disclosure also relates to treating, preventing, ameliorating, or reducing acute myeloid leukemia and other cancers.

### BACKGROUND

Acute myeloid leukemia (AML) is a clinically and molecularly heterogeneous disease that is classified using morphologie, immunophenotypic and genetic features. Sequencing of large patient cohorts has uncovered a complex mutational landscape in AML, but still fails to completely explain the biological and clinical heterogeneity in favorable and unfavorable groups. It has recently been identified that non-genetic molecular features, such as epigenetic modifications and patterns, are relatively underexplored characteristics involved in AML.

Epigenetic modifications are important for gene regulation and alterations to epigenetic programming are common in cancer. DNA methylation is a stable, yet reversible epigenetic modification involving the covalent addition of a methyl group to the 5′ carbon of cytosines in cytosine-guanine dinucleotides (CpG). The use of DNA methylation signatures for risk stratification improves the ability to predict clinical outcomes in the context of other well-described genetic, clinical, and demographic features.

Given the limitations described above, there is a need to utilize epigenetic patterns, such as for example DNA methylation, to identify, treat, and/or prevent specific disease states, such as AML and other cancers.

The compositions and methods disclosed herein address these needs.

### SUMMARY

The present disclosure provides methods of identifying epigenetic patterns associated with acute myeloid leukemia and other cancers. The present disclosure also provides kits and method of treating cancer by identifying epigenetic patterns associated with acute myeloid leukemia and other cancers.

In one aspect, disclosed herein is a method of treating a subject with cancer, the method comprising obtaining a tissue sample from the subject, extracting a nucleic acid from the tissue sample, analyzing an epigenetic pattern of the nucleic acid, comparing the epigenetic pattern from the subject to a control panel, categorizing the subject into an epitype selected from epitype 1, epitype 2, epitype 3, epitype 4, epitype 5, epitype 6, epitype 7, epitype 8, epitype 9, epitype 10, epitype 11, epitype 12, or epitype 13 based on the epigenetic pattern, and administering a treatment to the subject according to the at least one epitype.

In one aspect, disclosed herein is a method of identifying a specific disease state, wherein the disease state is associated with a given epigenetic pattern, the method comprising analyzing the epigenetic pattern in a subject without the specific disease or in one or more subjects at varying stages of disease, linking various disease states with epigenetic patterns, linking no disease state with epigenetic patterns, and developing epitypes based on the disease state and the epigenetic patterns.

In some embodiments, the epigenetic pattern comprises a methylation of a deoxyribonucleic acid (DNA) sequence. In some embodiments, the methylation comprises a hypermethylation or a hypomethylation. In some embodiments, the methylation occurs at a cytosine-phosphate-guanosine (CpG) island of the nucleic acid.

In some embodiments, the cancer is an acute myeloid leukemia (AML). In some embodiments, the treatment method comprises regular monitoring by a physician. In some embodiments, the treatment comprises a drug. In some embodiments, the drug is a Menin inhibitor.

In some embodiments, the subject retains a methylation pattern associated with a tumor genetic marker yet lacks the tumor genetic marker. In some embodiments, the genetic marker comprises FLT3-ITD, KMT2A, or NPM1.

In some embodiments, the thirteen epitypes are further divided into 4 superclusters (SC) selected from a transcription factor (TF)-SC, an MLL-SC, a NPM1-SC, or a stem-cell like (SL)-SC. In some embodiments, the TF-SC comprises epitype 1, epitype 2, epitype 3, or epitype 4. In some embodiments, the TF-SC comprises a disruption to one or more transcription factors (TFs). In some embodiments, the MLL-SC comprises epitype 5 or epitype 6. In some embodiments, the MLL-SC comprises a rearrangement of a KMT2A/MLL gene. In some embodiments, the NPM1-SC comprises epitype 7, epitype 8, epitype 9, or epitype 10. In some embodiments, the NPM1-SC comprises at least one NPM1 mutation. In some embodiments, the SL-SC comprises epitype 11, epitype 12, or epitype 13. In some embodiments, the SL-SC displays DNA methylation patterns similar to DNA methylation patterns in hematopoietic stem cells.

In some embodiments, the epigenetic pattern comprises a methylation of a deoxyribonucleic acid (DNA) sequence. In some embodiments, the disease state comprises progression, status, or severity of the disease.

In some embodiments, the hypomethylation occurs at a signal transducer and activator of transcription (STAT) gene.

In one aspect, disclosed herein is a kit for detecting an epigenetic modification of a deoxyribonucleic acid (DNA) sequence from a tissue sample. In some embodiments, the tissue sample is derived from a subject.

In some embodiments, the kit comprises a DNA denaturing reagent. In some embodiments, the kit comprises a DNA conversion reagent. In some embodiments, the DNA conversion reagent converts cytosine to thymine. In some embodiments, the kit comprises a binding buffer, a washing buffer, and an elution buffer.

In some embodiments, the epigenetic modification comprises a methylation modification. In some embodiments, the methylation modification occurs at a cytosine-phosphate-guanosine (CpG) island on a DNA molecule. In some embodiments, the methylation modification on the DNA molecule is further sequenced by methylation iPLEX (Me-iPLEX) technology.

### BRIEF DESCRIPTION OF FIGURES

The accompanying figures, which are incorporated in and constitute a part of this specification, illustrate several aspects described below.

FIGS. 1A 1B, IC, and ID show the training and calibration of the random forest classifier. FIG. 1A show the (left) raw random forest (RF) scores for epitype calls of 1,262 AML patients. The RF classifier was trained on 43 selected CpGs from Illumina array data on set samples with known (reference) epitype calls. 1,262 AML patients were assayed using the AML Me-iPLEX assay which served as test data. (right) Calibrated probability scores generated by multinomial logistic regression resulting in similar probability distributions across epitypes to allow for cross-class comparison. FIG. 1B shows the confusion matrix showing the results of internal cross validation of the random forest model. Internal cross validation of training set data resulted in an 87% probability of correct epitype classification. FIG. 1C shows the comparison of raw forest scores and calibrated probabilities in the training set. Calibration did not result in a reduction in concordance (AUC) while still improving the Brier score and log loss of the classifier (lower values represent higher accuracy). FIG. 1D shows the identification of the minimum class probability cutoff. To assess sample fit to an assigned epitype (class) we compared the sensitivity and specificity of RF calls in training samples from the Me-iPLEX assay that were correctly assigned to the same subtype when using epitype calls from Illumina array data. This was set to a probability cutoff that maintains 100% specificity (0.453), which resulted in a 94.5% sensitivity. Samples falling below this cut off were labeled as unclassifiable (157/1,262).

FIGS. 2A and 2B show the DNA methylation patterns across epitypes in reference training and test (Alliance cohort) samples. DNA methylation levels for 43 CpGs were averaged for all patients within each epitype and displayed as a heatmap. FIG. 2A shows he left heatmap shows CpG methylation levels derived from Illumina array data from 415 samples from the Beat AML and TCGA AML cohorts that serve as reference samples for training the classifier. FIG. 2B shows the right heatmap is the same CpGs (or a neighboring CpG) measured by the Me-iPLEX assay. Individual CpGs were clustered vertically using hierarchical clustering of the average CpG methylation values in the training set.

FIGS. 3A, 3B, and 3C show the classification of 1,262 AML patients using DNA methylation patterns. FIG. 3A shows the t-SNE plot generated using DNA methylation values for 43 CpGs determined using the AML Me-iPLEX assay. AML patients were assigned to 13 DNA methylation epitypes (colors) using a random forest classifier trained on reference epitype samples. The samples with probability scores below threshold were deemed unclassifiable (open circles). FIG. 3B shows the pie chart illustrating the relative proportions of patients classified per epitype and organization into one of four superclusters (SCs). Epitypes are represented by colors indicated in FIG. 3A. FIG. 3C shows the oncoprint displaying the genetic features of epitypes. Patients are grouped by epitype and ordered by the total number of observed genetic aberrations. Mutations and chromosomal aberrations are grouped by function and ordered by overall prevalence. Number of mutations per epitype is indicated.

FIGS. 4A, 4B, 4C, 4D, 4E, and 4F show the epiphenocopying of dominant mutations in epitypes and SHS signatures. FIG. 4A shows the oncoprint illustrating the proportion of E5 patients with the dominant t(v;11) mutation and mutations significantly enriched in patients lacking t(v;11) (X² test, P<0.05). FIGS. 4B and 4C show the oncoprints illustrating the same analysis for E8 exhibiting NPM1 mutations (FIG. 4B) and E12 patients exhibiting complex karyotype (FIG. 4C). For clarity, spliceosome genes enriched in E12 are shown separately in supplement. FIG. 4D shows the classification of the STAT hypomethylation signature (SHS) in 1,221 AML patients. Heatmap of the 29 CpGs that comprise the SHS signature across patients ranked by median DNA methylation value. Median DNA methylation value and dichotomization of SHS positive/negative patients is indicated (red line). FIG. 4E shows the scatterplot displaying the SHS median value versus the FLT3-ITD allelic ratio. The cutoff for SHS positivity (median<0.57) and FLT3-ITD+ (>0.5 allelic ratio) are indicated (red and grey dashed lines, respectively). The density of SHS median values is shown above. FIG. 4F shows the oncoprint of SHS+ patients showing FLT3-ITD and those with gene mutations that are significantly enriched in patients lacking FLT3-ITD (X² test, P<0.05).

FIGS. 5A and 5B show the analysis of spliceosome gene mutations in AML epitypes. FIG. 5A shows the histogram showing the frequency of the 5 most commonly mutated genes involving splicing in AML across 13 epitypes. Epitypes in the stem-like cluster (E11-13) exhibit spliceosome gene mutations in greater than ⅓ of patients. SF3B1 mutations are predominant in E12. FIG. 5B shows the oncoprint of patients in E12 showing those with complex karyotype and spliceosome gene mutations that are enriched in patients lacking complex karyotype. NRAS and WT1 mutations were also enriched in patients lacking complex karyotype and exhibited mutually exclusive patterns with splicing genes.

FIGS. 6A, 6B, and 6C show the classification of the STAT hypomethylation signature (SHS) in 1,221 AML patients. FIG. 6A show the concordance of SHS-positive classification from genome wide data¹⁰ with median SHS DNA methylation value (from Me-iPLEX analysis) in the same samples was assessed by receiver operating characteristic (ROC) curve analysis. A median SHS value of less than 0.57 (57% median methylation) most accurately identified SHS+ cases with a sensitivity of 0.90 and specificity of 0.94. FIG. 6B shows the proportion of SHS+ patients per epitype. FIG. 6C shows the pie charts displaying the number of FLT3-ITD and FLT3-TKD patients in SHS positive and negative groups.

FIGS. 7A and 7B show the overall survival of patients separated by epitype and within ELN risk groups. FIG. 7A shows the overall survival of all patients separated by epitype. FIG. 7B shows the overall survival of patients within the ELN favorable risk group separated by DNA methylation epitype supercluster. Patients in the MILL-SC display significantly shorter overall survival compared with TF-SC and NPM1-SC groups (P<0.0001 and P<0.05, respectively; log-rank test followed by Sidak adjustment for multiple comparisons).

FIGS. 8A, 8B, 8C, and 8D show the overall survival of patients within ELN risk groups separated by epitype. Epitypes were grouped into superclusters where necessary. Statistical differences were determined using log-rank tests followed by Sidak adjustment for multiple pairwise comparisons. FIG. 8A shows that within the ELN favorable risk group, patients in the MLL-SC displayed significantly shorter overall survival than epitypes E2 and E4 (P<0.01 for both). Comparisons to other individual epitypes did not reach statistical significance after adjusting for multiple comparisons. FIG. 8B shows that within the ELN intermediate risk group, patients in the TF-SC displayed significantly longer overall survival than epitypes E7 and E13 (P<0.05 and P<0.01, respectively). FIG. 8C shows that within the ELN adverse risk group, epitypes E12 and E13 displayed poorer overall survival than E11 (P<0.05). FIG. 8D shows the overall survival of all patients separated by SHS and FL73-ITD. Within FLT3-ITD-negative patients, those with SHS+ displayed poorer outcome (grey versus red lines; P<0.001). Patients positive for both SHS and FLT3-ITD (red line) displayed significantly inferior overall survival than all other groups, including versus SHS+/FLT3-ITD-(P<0.0001), SHS-/FLT3-ITD+ (P<0.001), and SHS+/FLT3-ITD-(P<0.001).

FIGS. 9A, 9B, 9C, and 9D show the importance of DNA methylation when combined with other markers in predicting clinical endpoints using a machine-learning model. FIG. 9A shows that the pie charts showing the relative importance of various classes of features included in models to predict overall survival. Top plot includes all standard features including, clinical, demographic, copy-number alterations (CNAs), fusions, and single-nucleotide variants (SNV) plus small insertion/deletions (Indels). The bottom plot in addition includes DNA methylation signatures. FIG. 9B shows that the volcano plot showing the association of all features when combined to predict overall survival. Colors represent feature class from FIG. 9A. Dashed and dotted lines represent FDR-adjusted significance levels of q<0.1 and q<0.05, respectively. FIG. 9C shows the stacked bar plot illustrating the relative importance of various feature classes for specific clinical endpoints when including DNA methylation. FIG. 9D shows the volcano plot showing the association of all features with attainment remission.

FIGS. 10A, 10B, 10C, 10D, and 10E show the additional analyses of combined features of AML patients using multi-stage, random-effects modeling. FIGS. 10A, 10B, 10C, and 10D shows the volcano plots showing the hazard ratio and P-value for all features when combined to predict (FIG. 10A) non-remission death, (FIG. 10B) non-relapse death, (FIG. 10C) relapse, and (FIG. 10D) post-relapse death endpoints. Colors represent feature class designations from FIG. 4. Dashed and dotted lines represent FDR-adjusted (Benjamini-Hochberg) q<0.1 and q<0.05, respectively. Individual features passing q<0.05 are labelled. Size of the circles represents the frequency of the feature among all patients. FIG. 10E shows the concordance between predicted and actual outcomes using internal cross-validation for all clinical endpoints examined. Concordance was analyzed separately using models that included and excluded DNA methylation features (epitype and SHS). Inclusion of DNA methylation features improved concordance in all endpoints examined.

FIGS. 11A, 11B, and 11C show the validation of the impact of DNA methylation on overall survival prediction using external sample cohorts. FIG. 11A shows the receiver operating characteristic (ROC) curve analysis of RFX model predicted versus actual overall survival at one year in the Beat AML cohort. The analysis was performed with and without DNA methylation information (red and blue curves, respectively) on all samples with available clinical, demographic, genetic and epigenetic data (n=207). FIGS. 11B and 11C show the concordance of RFX model predicted versus actual overall survival in the cohorts across various time points when including or excluding DNA methylation information. Analysis of the TCGA AML cohort was performed on all samples with available clinical, demographic, genetic and epigenetic data (n=178). The inclusion of DNA methylation information improved prediction accuracy in all cohorts and time points analyzed; (FIG. 11B) Beat AML, (FIG. 11C) TCGA AML.

FIGS. 12A, 12B, 12C, and 12D shows the epiphenocopying of favorable risk genetic markers redefines favorable risk AML patients. FIG. 12A shows the overall survival of patients within E4 separated by the presence or absence of CEBPA-dm. The E4 epiphenocopy group exhibits significantly more favorable outcome compared to intermediate and advanced ELN risk groups (P<0.0001; log-rank test with Sidak adjustment). FIG. 12B shows the overall survival of patients within E2,3 separated by the presence or absence of t(8;21) or inv(16). The E2,3 epiphenocopy group exhibits significantly more favorable outcome compared to intermediate and advanced ELN risk groups (P<0.0001; log-rank test with Sidak adjustment).

FIG. 12C shows the overall survival of patients with NPM/mutations separated by SHS and FLT3-ITD status. SHS-positive groups (red, black lines) performed significantly poorer than SHS-negative groups (green, grey lines) regardless of FLT3-ITD status, (P<0.0001; log-rank test with Sidak adjustment). FIG. 12D shows the patients assigned to the revised (M)-Favorable risk group demonstrate significantly better overall survival compared to patients formerly classified as ELN favorable but excluded due to unfavorable DNA methylation signatures (P<0.0001; log-rank test).

FIGS. 13A, 13B, 13C, and 13D show that the CEBPA DNA methylation and CEBPA single mutations do not underlie epiphenocopying of CEBPA-dm mutations. FIG. 13A shows the t-SNE plot of epitypes using Me-iPLEX DNA methylation data with CEBPA mutation status annotated across all samples. Dispersion of CEBPA single (monoallelic) mutations (CEBPA-sm) illustrates that CEBPA-sm are randomly distributed among and within epitypes. These findings are consistent with CEBPA-sm not associating with outcome risk.⁶ The frequency of CEBPA-sm within epitypes ranged in frequency from 0-4% of patients, similar to the overall rate of 4% observed in E4. The lack of enrichment of CEBPA-sm in E4 suggests that CEBPA-sm does not underlie E4 epiphenocopying. Epitypes are colored in the inset for reference. FIG. 13B shows the measurement of CEBPA promoter DNA methylation across all samples using the MassARRAY EpiTYPER assay. This analysis revealed high methylation levels were focused in E2 and E3 epitypes, consistent with previous observations in CBF AML.³⁹ Other epitypes exhibited either a low frequency (10-30% in E10-13) or a paucity (<10% in other epitypes) of hypermethylated patients, including E4. The lack of hypermethylation in E4 shows that CEBPA promoter DNA methylation does not underlie E4 epiphenocopying. The position of the MassARRAY amplicon targeting the CEBPA promoter is indicated in FIG. 13C. Five CpGs in the amplicon were averaged per sample. FIG. 13C shows the high-resolution DNA methylation heatmaps showing methylation of single CpGs in individual E4 patients separated by CEBPA mutation status. DNA methylation was measured using the MassARRAY EpiTYPER assay, and the position of amplicons relative to CEBPA and regional conservation between species are shown. Of the E4 epiphenocopy patients (CEBPA wild-type and CEBPA-sm), only 3/12 displayed CEBPA promoter hypermethylation, with 2/3 CEBPA-sm patients showing promoter hypermethylation (right panel). Further evaluation of an enhancer region important for CEBPA expression in myeloid cells⁴⁰ (+42-kb, left panels), failed to detect hypermethylation in all samples tested. FIG. 13D shows that ⅔ E4 patients with CEBPA-sm showed CEBPA promoter DNA hypermethylation, the interaction between CEBPA promoter methylation CEBPA mutation status was further investigated. We did not detect a difference in the degree of CEBPA promoter methylation between CEBPA-sm and CEBPA wild-type patients, indicating that hypermethylation is not a common mechanism driving functional bi-allelic CEBPA loss in the presence of CEBPA-sm.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A “control” is an alternative subject or sample used in an experiment for comparison purposes. A control can be “positive” or “negative.”

As used herein, “categorize”, “categorized”, categorizing”, and any grammatical variations thereof, refers to an act of placing at least two or more entities, such as a subject, into a particular class or group based on a similar feature (such as, for example a specific epigenetic pattern), object, trait, or characteristic. It should be understood that “assort”, “classify”, “compartmentalize”, “rank”, “sort”, “group”, or “distribute” can be used interchangeably with grammatical variations of “categorize”.

As used herein, “monitoring” refers to the actions of observing and checking the progress or quality of a treatment or procedure over a period of time. “Monitoring” also refers to observing the course of a disease or condition, such as a cancer, over a period of time.

As used herein, “diagnose”, “diagnosed”, “diagnosing”, and any grammatical variations thereof as used herein, refers to the act of process of identifying the nature of an illness, disease, disorder, or condition in a subject by examination or monitoring of symptoms.

As used herein, the term “buffer” refers to a solution consisting of a mixture of acid and its conjugate base, or vice versa. The solution is used as a means of keeping the pH at a nearly constant range to be used in a wide variety of chemical and biological applications.

As used herein, the term “drug” refers to a compound or composition that is used as a medicine to have a physiological and/or psychological effect when introduced into the body of a subject. A “prodrug” refers to a compound or composition that after administration or ingestion is metabolized into a pharmaceutically active drug. Prodrugs can also be viewed as compounds or compositions containing specialized nontoxic protective properties used in a transient manner to alter or eliminate undesirable properties of the active drug.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less. A “variant” or a “derivative” of a particular inhibitor may be defined as a chemical or molecular compound having at least 50% identity to a parent or original inhibitor. In some embodiments a variant inhibitor may show, for example, at least 60%, at least 70%, at least 80%, at least 90%, at least 91%, at least 92%, at least 93%, at least 94%, at least 95%, at least 96%, at least 97%, at least 98%, or at least 99% or greater identity relative to a reference parent or original inhibitor.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated.

As used herein, “epigenetic modification” refers to the heritable genetic changes the affect gene expression activity without altering the DNA or RNA sequence. These genetic changes include, but are not limited to DNA or RNA methylation and histone modifications (i.e.: methylation and/or acetylation) that alter DNA or RNA accessibility and structure, thereby regulating gene expression patterns.

The term “methylation” refers to the chemical modification to a molecule by adding a methyl group on a DNA, RNA, or protein molecule. This modification is usually performed by enzymes to regulate gene expression, protein function, and RNA processing.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material. A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

As used herein, a “mutation” refers to changing the structure of a gene, resulting in a variant form that may be transmitted to later generations. A mutation is caused by the alteration of single nucleotides in DNA, or the deletion, insertion, or rearrangement of larger sections of genes. A mutation can lead to the expression of a protein that has been changed physically or functionally leading to lethality, non-lethal dysfunction effects, or no effects.

As used herein, “extract”, “extracting”, “extracted” or any other variations refers to obtaining a resource, substance, or data from an initial source, for example, to include, but not limited to an image, sample, or medical history, wherein the initial source provides further information about the health, condition, and status of a subject or patient.

Methods of Identifying, Treating, and/or Preventing a Cancer

Epigenetics is the study of non-sequence information of chromosomal DNA during cell division and differentiation. The molecular basis of epigenetics is complex and involves modifications of the activation and inactivation of certain genes. Additionally, the chromatin proteins associated with DNA may be activated or silenced. Epigenetic changes are preserved when cells divide. Most epigenetic changes occur within the course of one individual organism's lifetime, but some epigenetic changes are inherited from one generation to the next. One example of an epigenetic modification includes DNA methylation, which refers to a covalent modification of a cytosine nucleotide. In particular, the addition of one or more methyl groups to a cytosine nucleotide in a DNA sequence, thus converting the cytosine to a 5-methylcytosine. DNA methylation plays an important role in regulating expression of genes. Thus, abnormal DNA methylation is one of the mechanisms known to underlie the changes observed in cancers.

Cancers have historically been linked to genetic changes such as DNA mutations. Evidence now indicates that a large number of cancers originate, not from mutations, but from epigenetic changes such as inappropriate DNA methylation. Non-limiting examples of inappropriate methylation includes hypermethylation and hypomethylation. As used herein, “hypermethylation” refers to an increased level or occurrence of methylation to cytosine, and sometimes adenosine, nucleotides relative to a normal state of methylation. As used herein, “hypomethylation” refers to a decreased level or occurrence of methylation to cytosine, and sometimes adenosine, nucleotides relative to a normal state of methylation. In some instances, hypermethylation of genes results in inhibition of expression of tumor suppressor genes or DNA repair genes, allowing for cancers to develop. In other instances, hypomethylation of genes modulates expression, which also contributes to cancer development.

Acute myeloid leukemia (AML) is an aggressive hematological cancer that has been characterized with dysregulated epigenetic mechanisms, which are initiated by recurrent translocations and/or mutations in transcription factors and chromatin regulators. Because of the heterogenous nature of AML, AML patients classified based on risk stratification groups to ensure optimal treatment strategies. However, such risk stratification groups do not account for epigenetic modifications to genes associated with AML.

Thus, the present disclosure provides methods of identifying epigenetic patterns associated with acute myeloid leukemia and other cancers. The present disclosure also provides kits and method of treating cancer by identifying epigenetic patterns associated with acute myeloid leukemia and other cancers.

In one aspect, disclosed herein is a method of treating a subject with cancer, the method comprising obtaining a tissue sample from the subject, extracting a nucleic acid from the tissue sample, analyzing an epigenetic pattern of the nucleic acid, comparing the epigenetic pattern from the subject to a control panel, categorizing the subject into an epitype selected from epitype 1, epitype 2, epitype 3, epitype 4, epitype 5, epitype 6, epitype 7, epitype 8, epitype 9, epitype 10, epitype 11, epitype 12, or epitype 13 based on the epigenetic pattern, and administering a treatment to the subject according to the at least one epitype. As used herein, an “epitype” refers to an epigenetic modification to a specific gene or class of genes.

In one aspect, disclosed herein is a method of identifying a specific disease state, wherein the disease state is associated with a given epigenetic pattern, the method comprising analyzing the epigenetic pattern in a subject without the specific disease or in one or more subjects at varying stages of disease, linking various disease states with epigenetic patterns, linking no disease state with epigenetic patterns, and developing epitypes based on the disease state and the epigenetic patterns. In some embodiments, the specific disease comprises a cancer. In some embodiments, the disease state comprises progression, status, or severity of the disease.

In some embodiments, the method comprises 13 epitypes. In some embodiments, the method comprises less than 13 epitypes. In some embodiments, the method comprises 1, 2, 3, 4, 5, 6, 7, 8, 9, 10, 11, or 12 epitypes. In some embodiments, the method comprises more than 13 epitypes. In some embodiments, the method comprises 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more epitypes.

In some embodiments, the tissue sample comprises a blood sample. In some embodiments, the tissue sample comprises a tissue biopsy. In some embodiments, the tissue sample comprises a urine sample. In some embodiments, the tissue sample comprises a fecal sample.

In some embodiments, the epigenetic pattern comprises a methylation of a deoxyribonucleic acid (DNA) sequence. In some embodiments, the methylation comprises a hypermethylation or a bypomethylation. In some embodiments, the methylation occurs at a cytosine nucleotide. In some embodiments, the methylation occurs at a cytosine-phosphate-guanosine (CpG) island of the nucleic acid. In some embodiments, the methylation occurs at an adenosine nucleotide.

In some embodiments, the cancer comprises an acute myeloid leukemia (AML). In some embodiments, the AML comprises B-cell AML. In some embodiments, the AML comprises T-cell AML. In some embodiments, the cancer includes, but is not limited to acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, the treatment method comprises regular monitoring by a physician. In some embodiments, the treatment comprises a drug. In some embodiments, the drug is a Menin inhibitor. In some embodiments, the treatment further comprises an anti-cancer agent selected from interferons, cytokines (e.g., tumor necrosis factor, interferon α, interferon Y), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DMI, AVASTIN (bevacizumab), ERBITUX (cetuximab), VECTIBIX (panitumumab), RITUXAN (rituximab), BEXXAR (tositumomab)), anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide BC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHER inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin, aminopterin, and hexamethyl melamine.

In some embodiments, the subject retains a methylation pattern associated with a tumor genetic marker yet lacks the tumor genetic marker. In some embodiments, the genetic marker comprises FLT3-ITD, KMT2A, or NPM1.

It should be understood that the epitype of any preceding aspect comprises methylation at least one gene including, but not limited to ZSCAN25, HCCA2, RGS12, HOXB3.1, BEND7, ALS2CL, HMGA1, HOXB-AS3.2, PPPIR18, DNMT3A.2, MLLT10, DNMT3A.1, PRKAG2, TM4SF19, CCDC9B, ZNF438, MED13L, CHML, TULP4, ZZEF, ACOT7, LRPAP1, PALM.2, PALM.1, ESRP2, MEF2B, REC8, PDYN-AS1, GIMAP7, XXYLT1, HIVEP3, WT1, CD34.2, CD34.1, AIM2, A4GALT, CTTN, CELF2, HOXB3.2.2, HOXB3.2.1, and HOXB3.3. In some embodiments, the epitype of any preceding aspect comprises methylation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genes, including but not limited to ZSCAN25, HCCA2, RGS12, HOXB3.1, BEND7, ALS2CL, HMGA1, HOXB-AS3.2, PPPIR18, DNMT3A.2, MLLT10, DNMT3A.1, PRKAG2, TM4SF19, CCDC9B, ZNF438, MED13L, CHML, TULP4, ZZEF, ACOT7, LRPAP1, PALM.2, PALM.1, ESRP2, MEF2B, REC8, PDYN-AS1, GIMAP7, XXYLT1, HIVEP3, WT1, CD34.2, CD34.1, AIM2, A4GALT, CTTN, CELF2, HOXB3.2.2, HOXB3.2.1, or HOXB3.3. In some embodiments, the at least one gene including, but not limited to ZSCAN25, HCCA2, RGS12, HOXB3.1, BEND7, ALS2CL, HMGA1, HOXB-AS3.2, PPPIR18, DNMT3A.2, MLLT10, DNMT3A.1, PRKAG2, TM4SF19, CCDC9B, ZNF438, MED13L, CHML, TULP4, ZZEF, ACOT7, LRPAP1, PALM.2, PALM.1, ESRP2, MEF2B, REC8, PDYN-AS1, GIMAP7, XXYLT1, HIVEP3, WT1, CD34.2, CD34.1, AIM2, A4GALT, CTTN, CELF2, HOXB3.2.2, HOXB3.2.1, and HOXB3.3 comprises a nonlimiting percentage of methylation relative to methylation in a non-cancerous sample. In some embodiments, the at least one gene including, but not limited to ZSCAN25, HCCA2, RGS12, HOXB3.1, BEND7, ALS2CL, HMGA1, HOXB-AS3.2, PPPIR18, DNMT3A.2, MLLT10, DNMT3A.1, PRKAG2, TM4SF19, CCDC9B, ZNF438, MED13L, CHML, TULP4, ZZEF, ACOT7, LRPAP1, PALM.2, PALM.1, ESRP2, MEF2B, REC8, PDYN-AS1, GIMAP7, XXYLT1, HIVEP3, WT1, CD34.2, CD34.1, AIM2, A4GALT, CTTN, CELF2, HOXB3.2.2, HOXB3.2.1, and HOXB3.3 comprises about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more methylation relative to methylation in a non-cancerous sample.

Regarding epitype 1, the following genes comprise a nonlimiting percentage range of methylation relative to methylation in a non-cancerous sample. ZSCAN25 comprises between about 5%-15% methylation, HCCA comprises between about 5%-15% methylation, RGS12 comprises between about 5%-15% methylation, HOXB3.1 comprises between about 5%-20% methylation, BEND7 comprises between about 0%-20% methylation, ALS2CL comprises between about 0%-50% methylation, HMGA1 comprises between about 10%-50% methylation, HOXB-AS3.2 comprises between about 5%-10% methylation, PPPIR18 comprises between about 50%-80% methylation, DNMT3A.2 comprises between about 25%-35% methylation, MLLT10 comprises between about 35%-55% methylation, DNMT3A.1 comprises between about 15%-25% methylation, PRKAG2 comprises between about 10%-20% methylation, TM4SF19 comprises between bout 25%-50% methylation, CCDC9B comprises between about 50%-100% methylation, ZNF438 comprises between about 90%-100% methylation, MED13L comprises between about 90%-100% methylation, CHML comprises between about 85%-100% methylation, TULP4 comprises between about 90%-100% methylation, ZZEF comprises between about 90%-100% methylation, ACOT7 comprises between about 90%-100% methylation, LRPAP1 comprises between about 5-10% methylation, PALM.2 comprises between about 50%-100% methylation, PALM.1 comprises between about 50%-100% methylation, ESRP2 comprises between about 40%-50% methylation, MEF2B comprises between about 10-25% methylation, REC8 comprises between about 50%-85% methylation, PDYN-AS1 comprises between about 25%-100% methylation, GI MAP7 comprises between about 50%-75% methylation, XXYLT1 comprises between about 80%-90% methylation, HIVEP3 50%-85% methylation, WT1 comprises between about 70%-90% methylation, CD34.2 comprises between about 80%-100% methylation, CD34.1 comprises between about 80%-100% methylation, AIM2 comprises between about 15%-100% methylation, A4GALT comprises between about 20%-40% methylation, CTTN comprises between about 30%-75% methylation, CELF2 comprises between about 80%-100% methylation, HOXB-AS3.1 comprises between about 75%-100% methylation, MIRLET7BHG comprises between about 90%-100% methylation, HOXB3.2.2 comprises between about 80%-100% methylation, HOXB3.2.1 comprises between about 50%-100% methylation, and/or HOXB3.3 comprises between about 80%-90% methylation. It has been further contemplated that epitype 1 can comprise additional genes with a non-limiting range of methylation relative to methylation in a non-cancerous sample.

Regarding epitype 2, the following genes comprise a nonlimiting percentage range of methylation relative to methylation in a non-cancerous sample. ZSCAN25 comprises between about 10%-25% methylation, HCCA comprises between about 5%-20% methylation, RGS12 comprises between about 5%-15% methylation, HOXB3.1 comprises between about 10%-20% methylation, BEND7 comprises between about 0%-10% methylation, ALS2CL comprises between about 0%-10% methylation, HMGA1 comprises between about 0%-10% methylation, HOXB-AS3.2 comprises between about 0%-10% methylation, PPPIR18 comprises between about 0%-10% methylation, DNMT3A.2 comprises between about 80%-90% methylation, MLLT10 comprises between about 85%-100% methylation, DNMT3A.1 comprises between about 70%-80% methylation, PRKAG2 comprises between about 25%-35% methylation, TM4SF19 comprises between about 50%-70% methylation, CCDC9B comprises between about 60%-80% methylation, ZNF438 comprises between about 85%-100% methylation, MED13L comprises between about 90%-100% methylation, CHML comprises between about 85%-100% methylation, TULP4 comprises between about 50%-70% methylation, ZZEF comprises between about 10%-20% methylation, ACOT7 comprises between about 15%-30% methylation, LRPAP1 comprises between about 15%-45% methylation, PALM.2 comprises between about 25%-35% methylation, PALM.1 comprises between about 10%-30% methylation, ESRP2 comprises between about 20%-30% methylation, MEF2B comprises between about 5%-15% methylation, REC8 comprises between about 60%-75% methylation, PDYN-AS1 comprises between about 50%-60% methylation, GI MAP7 comprises between about 70%-90% methylation, XXYLT1 comprises between about 60%-80% methylation, HIVEP3 comprises between about 50%-70% methylation, WT1 comprises between about 25%-35% methylation, CD34.2 comprises between about 0%-15% methylation, CD34.1 comprises between about 15%-30% methylation, AIM2 comprises between about 40%-90% methylation, A4GALT comprises between about 15%-25% methylation, CTTN comprises between about 80%-100% methylation, CELF2 comprises between about 90%-100% methylation, HOXB-AS3.1 comprises between about 70%-80% methylation, MI RLET7BHG comprises between about 90%-100% methylation, HOXB3.2.2 comprises between about 85%-100% methylation, HOXB3.2.1 comprises between about 85%-100% methylation, and/or HOXB3.3 comprises between about 70%-95% methylation. It has been further contemplated that epitype 2 can comprise additional genes with a non-limiting range of methylation relative to methylation in a non-cancerous sample.

Regarding epitype 3, the following genes comprise a nonlimiting percentage range of methylation relative to methylation in a non-cancerous sample. ZSCAN25 comprises between about 10%-20% methylation, HCCA comprises between about 70%-80% methylation, RGS12 comprises between about 5%-15% methylation, HOXB3.1 comprises between about 70%-90% methylation, BEND7 comprises between about 0%-10% methylation, ALS2CL comprises between about 0%-15% methylation, HMGA1 comprises between about 0%-10% methylation, HOXB-AS3.2 comprises between about 0%-10% methylation, PPPIR18 comprises between about 0%-5% methylation, DNMT3A.2 comprises between about 60%-70% methylation, MLLT10 comprises between about 85%-95% methylation, DNMT3A.1 comprises between about 40%-60% methylation, PRKAG2 comprises between about 80%-90% methylation, TM4SF19 comprises between about 10%-40% methylation, CCDC9B comprises between about 10%-25% methylation, ZNF438 comprises between about 80%-90% methylation, MED13L comprises between about 90%-100% methylation, CHML comprises between about 80%-90% methylation, TULP4 comprises between about 85%-95% methylation, ZZEF comprises between about 90%-100% methylation, ACOT7 comprises between about 90%-100% methylation, LRPAP1 comprises between about 10%-40% methylation, PALM.2 comprises between about 10%-25% methylation, PALM.1 comprises between about 10%-20% methylation, ESRP2 comprises between about 20%-30% methylation, MEF2B comprises between about 5%-15% methylation, REC8 comprises between about 10%-30% methylation, PDYN-AS1 comprises between about 10%-25% methylation, GI MAP7 comprises between about 40%-50% methylation, XXYLT1 comprises between about 55%-80% methylation, HIVEP3 comprises between about 70%-85% methylation, WT1 comprises between about 70%-80% methylation, CD34.2 comprises between about 0%-10% methylation, CD34.1 comprises between about 20%-30% methylation, AIM2 comprises between about 50%-90% methylation, A4GALT comprises between about 15%-30% methylation, CTTN comprises between about 15%-25% methylation, CELF2 comprises between about 90%-95% methylation, HOXB-AS3.1 comprises between about 75%-85% methylation, MI RLET7BHG comprises between about 90%-100% methylation, HOXB3.2.2 comprises between about 85%-100% methylation, HOXB3.2.1 comprises between about 90%-100% methylation, and/or HOXB3.3 comprises between about 85%-100% methylation. It has been further contemplated that epitype 3 can comprise additional genes with a non-limiting range of methylation relative to methylation in a non-cancerous sample.

Regarding epitype 4, the following genes comprise a nonlimiting percentage range of methylation relative to methylation in a non-cancerous sample. ZSCAN25 comprises between about 5%-10% methylation, HCCA comprises between about 5%-10% methylation, RGS12 comprises between about 10%-15% methylation, HOXB3.1 comprises between about 15%-30% methylation, BEND7 comprises between about 0%-5% methylation, ALS2CL comprises between about 20%-30% methylation, HMGA1 comprises between about 10%-30% methylation, HOXB-AS3.2 comprises between about 0%-10% methylation, PPPIR18 comprises between about 0%-5% methylation, DNMT3A.2 comprises between about 90%-100% methylation, MLLT10 comprises between about 90%-100% methylation, DNMT3A.1 comprises between about 85%-95% methylation, PRKAG2 comprises between about 85%-100% methylation, TM4SF19 comprises between about 80%-95% methylation, CCDC9B comprises between about 80%-90% methylation, ZNF438 comprises between about 90%-100% methylation, MED13L comprises between about 90%-100% methylation, CHML comprises between about 80%-90% methylation, TULP4 comprises between about 15%-25% methylation, ZZEF comprises between about 90%-100% methylation, ACOT7 comprises between about 80%-90% methylation, LRPAP1 comprises between about 20%-50% methylation, PALM.2 comprises between about 70%-80% methylation, PALM.1 comprises between about 50%-80% methylation, ESRP2 comprises between about 75%-85% methylation, MEF2B comprises between about 65%-75% methylation, REC8 comprises between about 65%-75% methylation, PDYN-AS1 comprises between about 70%-80% methylation, GI MAP7 comprises between about 70%-80% methylation, XXYLT1 comprises between about 60%-90% methylation, HIVEP3 comprises between about 75%-85% methylation, WT1 comprises between about 25%-30% methylation, CD34.2 comprises between about 10%-25% methylation, CD34.1 comprises between about 15%-25% methylation, AIM2 comprises between about 50%-90% methylation, A4GALT comprises between about 70%-80% methylation, CTTN comprises between about 75%-90% methylation, CELF2 comprises between about 90%-100% methylation, HOXB-AS3.1 comprises between about 75%-85% methylation, MI RLET7BHG comprises between about 90%-95% methylation, HOXB3.2.2 comprises between about 85%-95% methylation, HOXB3.2.1 comprises between about 85%-95% methylation, and/or HOXB3.3 comprises between about 75%-85% methylation. It has been further contemplated that epitype 4 can comprise additional genes with a non-limiting range of methylation relative to methylation in a non-cancerous sample.

Regarding epitype 5, the following genes comprise a nonlimiting percentage range of methylation relative to methylation in a non-cancerous sample. ZSCAN25 comprises between about 35%-45% methylation, HCCA comprises between about 10%-20% methylation, RGS12 comprises between about 15%-20% methylation, HOXB3.1 comprises between about 5%-20% methylation, BEND7 comprises between about 0%-10% methylation, ALS2CL comprises between about 0%-10% methylation, HMGA1 comprises between about 5%-15% methylation, HOXB-AS3.2 comprises between about 0%-10% methylation, PPPIR18 comprises between about 0%-5% methylation, DNMT3A.2 comprises between about 50%-65% methylation, MLLT10 comprises between about 50%-70% methylation, DNMT3A.1 comprises between about 30%-45% methylation, PRKAG2 comprises between about 40%-60% methylation, TM4SF19 comprises between about 50%-70% methylation, CCDC9B comprises between about 70%-80% methylation, ZNF438 comprises between about 45%-55% methylation, MED13L comprises between about 30%-70% methylation, CHML comprises between about 30%-45% methylation, TULP4 comprises between about 70%-80% methylation, ZZEF comprises between about 90%-100% methylation, ACOT7 comprises between about 90%-100% methylation, LRPAP1 comprises between about 80%-100% methylation, PALM.2 comprises between about 40%-50% methylation, PALM.1 comprises between about 30%-50% methylation, ESRP2 comprises between about 10%-25% methylation, MEF2B comprises between about 0%-10% methylation, REC8 comprises between about 10%-25% methylation, PDYN-AS1 comprises between about 5%-25% methylation, GI MAP7 comprises between about 25%-30% methylation, XXYLT1 comprises between about 25%-40% methylation, HIVEP3 comprises between about 20%-40% methylation, WT1 comprises between about 5%-20% methylation, CD34.2 comprises between about 20%-40% methylation, CD34.1 comprises between about 40%-60% methylation, AIM2 comprises between about 30%-55% methylation, A4GALT comprises between about 30%-40% methylation, CTTN comprises between about 40%-50% methylation, CELF2 comprises between about 20%-40% methylation, HOXB-AS3.1 comprises between about 45%-60% methylation, MI RLET7BHG comprises between about 30%-40% methylation, HOXB3.2.2 comprises between about 60%-70% methylation, HOXB3.2.1 comprises between about 55%-75% methylation, and/or HOXB3.3 comprises between about 40%-55% methylation. It has been further contemplated that epitype 5 can comprise additional genes with a non-limiting range of methylation relative to methylation in a non-cancerous sample.

Regarding epitype 6, the following genes comprise a nonlimiting percentage range of methylation relative to methylation in a non-cancerous sample. ZSCAN25 comprises between about 10%-20% methylation, HCCA comprises between about 5%-15% methylation, RGS12 comprises between about 20%-30% methylation, HOXB3.1 comprises between about 30%-95% methylation, BEND7 comprises between about 0%-10% methylation, ALS2CL comprises between about 10%-20% methylation, HMGA1 comprises between about 10%-30% methylation, HOXB-AS3.2 comprises between about 0%-60% methylation, PPPIR18 comprises between about 0%-10% methylation, DNMT3A.2 comprises between about 60%-90% methylation, MLLT10 comprises between about 60%-90% methylation, DNMT3A.1 comprises between about 50%-80% methylation, PRKAG2 comprises between about 55%-95% methylation, TM4SF19 comprises between about 70%-95% methylation, CCDC9B comprises between about 80%-95% methylation, ZNF438 comprises between about 90%-100% methylation, MED13L comprises between about 80%-100% methylation, CHML comprises between about 70%-85% methylation, TULP4 comprises between about 85%-95% methylation, ZZEF comprises between about 90%-100% methylation, ACOT7 comprises between about 90%-100% methylation, LRPAP1 comprises between about 90%-100% methylation, PALM.2 comprises between about 70%-85% methylation, PALM.1 comprises between about 60%-85% methylation, ESRP2 comprises between about 50%-90% methylation, MEF2B comprises between about 20%-80% methylation, REC8 comprises between about 30%-80% methylation, PDYN-AS1 comprises between about 50%-85% methylation, GI MAP7 comprises between about 65%-90% methylation, XXYLT1 comprises between about 605-90% methylation, HIVEP3 comprises between about 605-90% methylation, WT1 comprises between about 60%-85% methylation, CD34.2 comprises between about 20%-70% methylation, CD34.1 comprises between about 40%-75% methylation, AIM2 comprises between about 30%-60% methylation, A4GALT comprises between about 20%-40% methylation, CTTN comprises between about 20%-40% methylation, CELF2 comprises between about 20%-70% methylation, HOXB-AS3.1 comprises between about 30%-70% methylation, MI RLET7BHG comprises between about 60%-90% methylation, HOXB3.2.2 comprises between about 90%-95% methylation, HOXB3.2.1 comprises between about 85%-100% methylation, and/or HOXB3.3 comprises between about 80%-90% methylation. It has been further contemplated that epitype 6 can comprise additional genes with a non-limiting range of methylation relative to methylation in a non-cancerous sample.

Regarding epitype 7, the following genes comprise a nonlimiting percentage range of methylation relative to methylation in a non-cancerous sample. ZSCAN25 comprises between about 5%-15% methylation, HCCA comprises between about 5%-15% methylation, RGS12 comprises between about 10%-15% methylation, HOXB3.1 comprises between about 5%-10% methylation, BEND7 comprises between about 0%-5% methylation, ALS2CL comprises between about 0%-10% methylation, HMGA1 comprises between about 5%-15% methylation, HOXB-AS3.2 comprises between about 10%-20% methylation, PPPIR18 comprises between about 0%-5% methylation, DNMT3A.2 comprises between about 75%-85% methylation, MLLT10 comprises between about 65%-85% methylation, DNMT3A.1 comprises between about 55%-65% methylation, PRKAG2 comprises between about 85%-95% methylation, TM4SF19 comprises between about 70%-80% methylation, CCDC9B comprises between about 80%-90% methylation, ZNF438 comprises between about 85%-95% methylation, MED13L comprises between about 80%-90% methylation, CHML comprises between about 65%-75% methylation, TULP4 comprises between about 80%-90% methylation, ZZEF comprises between about 95%-100% methylation, ACOT7 comprises between about 90%-100% methylation, LRPAP1 comprises between about 90%-100% methylation, PALM.2 comprises between about 10%-25% methylation, PALM.1 comprises between about 10%-20% methylation, ESRP2 comprises between about 10%-20% methylation, MEF2B comprises between about 5%-15% methylation, REC8 comprises between about 15%-25% methylation, PDYN-AS1 comprises between about 35%-45% methylation, GI MAP7 comprises between about 70%-80% methylation, XXYLT1 comprises between about 40%-70% methylation, HIVEP3 comprises between about 50%-60% between about 60%-70% methylation, CD34.2 comprises between about 55%-75% methylation, CD34.1 comprises between about 80%-90% methylation, AIM2 comprises between about 5%-60% methylation, A4GALT comprises between about 15%-25% methylation, CTTN comprises between about 10%-30% methylation, CELF2 comprises between about 10%-20% methylation, HOXB-AS3.1 comprises between about 5%-15% methylation, MI RLET7BHG comprises between about 5%-15% methylation, HOXB3.2.2 comprises between about 10%-20% methylation, HOXB3.2.1 comprises between about 10%-20% methylation, and/or HOXB3.3 comprises between about 5%-10% methylation. It has been further contemplated that epitype 7 can comprise additional genes with a non-limiting range of methylation relative to methylation in a non-cancerous sample.

Regarding epitype 8, the following genes comprise a nonlimiting percentage range of methylation relative to methylation in a non-cancerous sample. ZSCAN25 comprises between about 5%-15% methylation, HCCA comprises between about 5%-15% methylation, RGS12 comprises between about 20%-30% methylation, HOXB3.1 comprises between about 10%-25% methylation, BEND7 comprises between about 0%-10% methylation, ALS2CL comprises between about 0%-20% methylation, HMGA1 comprises between about 15%-25% methylation, HOXB-AS3.2 comprises between about 50%-70% methylation, PPPIR18 comprises between about 0%-10% methylation, DNMT3A.2 comprises between about 80%-100% methylation, MLLT10 comprises between about 80%-100% methylation, DNMT3A.1 comprises between about 70%-80% methylation, PRKAG2 comprises between about 90%-100% methylation, TM4SF19 comprises between about 70%-90% methylation, CCDC9B comprises between about 80%-90% methylation, ZNF438 comprises between about 90%-100% methylation, MED13L comprises between about 90%-100% methylation, CHML comprises between about 80%-90% methylation, TULP4 comprises between about 85%-100% methylation, ZZEF comprises between about 95%-100% methylation, ACOT7 comprises between about 90%-100% methylation, LRPAP1 comprises between about 90%-100% methylation, PALM.2 comprises between about 40%-60% methylation, PALM.1 comprises between about 40%-55% methylation, ESRP2 comprises between about 75%-85% methylation, MEF2B comprises between about 60%-70% methylation, REC8 comprises between about 60%-75% methylation, PDYN-AS1 comprises between about 75%-90% methylation, GI MAP7 comprises between about 80%-90% methylation, XXYLT1 comprises between about 60%-90% methylation, HIVEP3 comprises between about 80%-85% methylation, WT1 comprises between about 75%-85% methylation, CD34.2 comprises between about 60%-75% methylation, CD34.1 comprises between about 75%-85% methylation, AIM2 comprises between about 10%-60% methylation, A4GALT comprises between about 15%-25% methylation, CTTN comprises between about 10%-25% methylation, CELF2 comprises between about 20%-25% methylation, HOXB-AS3.1 comprises between about 10%-25% methylation, MI RLET7BHG comprises between about 10%-30% methylation, HOXB3.2.2 comprises between about 20%-40% methylation, HOXB3.2.1 comprises between about 20%-35% methylation, and/or HOXB3.3 comprises between about 15%-30% methylation. It has been further contemplated that epitype 8 can comprise additional genes with a non-limiting range of methylation relative to methylation in a non-cancerous sample.

Regarding epitype 9, the following genes comprise a nonlimiting percentage range of methylation relative to methylation in a non-cancerous sample. ZSCAN25 comprises between about 60%-85% methylation, HCCA comprises between about 50%-65% methylation, RGS12 comprises between about 5%-20% methylation, HOXB3.1 comprises between about 5%-15% methylation, BEND7 comprises between about 20%-30% methylation, ALS2CL comprises between about 5%-30% methylation, HMGA1 comprises between about 40%-50% methylation, HOXB-AS3.2 comprises between about 35%-50% methylation, PPPIR18 comprises between about 15%-25% methylation, DNMT3A.2 comprises between about 90%-100% methylation, MLLT10 comprises between about 90%-100% methylation, DNMT3A.1 comprises between about 85%-95% methylation, PRKAG2 comprises between about 90%-100% methylation, TM4SF19 comprises between about 80%-95% methylation, CCDC9B comprises between about 85%-95% methylation, ZNF438 comprises between about 85%-95% methylation, MED13L comprises between about 90%-100% methylation, CHML comprises between about 90%-100% methylation, TULP4 comprises between about 90%-100% methylation, ZZEF comprises between about 90%-100% methylation, ACOT7 comprises between about 90%-100% methylation, LRPAP1 comprises between about 90%-100% methylation, PALM.2 comprises between about 90%-100% methylation, PALM.1 comprises between about 85%-95% methylation, ESRP2 comprises between about 30%-50% methylation, MEF2B comprises between about 15%-20% methylation, REC8 comprises between about 20%-30% methylation, PDYN-AS1 comprises between about 75%-85% methylation, GI MAP7 comprises between about 70%-80% methylation, XXYLT1 comprises between about 80%-90% methylation, HIVEP3 comprises between about 45%-60% methylation, WT1 comprises between about 70%-80% methylation, CD34.2 comprises between about 80%-90% methylation, CD34.1 comprises between about 90%-100% methylation, AIM2 comprises between about 30%-60% methylation, A4GALT comprises between about 50%-60% methylation, CTTN comprises between about 50%-80% methylation, CELF2 comprises between about 10%-20% methylation, HOXB-AS3.1 comprises between about 10%-20% methylation, MI RLET7BHG comprises between about 5%-30% methylation, HOXB3.2.2 comprises between about 20%-30% methylation, HOXB3.2.1 comprises between about 20%-40% methylation, and/or HOXB3.3 comprises between about 15%-30% methylation. It has been further contemplated that epitype 9 can comprise additional genes with a non-limiting range of methylation relative to methylation in a non-cancerous sample.

Regarding epitype 10, the following genes comprise a nonlimiting percentage range of methylation relative to methylation in a non-cancerous sample. ZSCAN25 comprises between about 75%-90% methylation, HCCA comprises between about 80%-90% methylation, RGS12 comprises between about 30%-60% methylation, HOXB3.1 comprises between about 40%-60% methylation, BEND7 comprises between about 25%-35% methylation, ALS2CL comprises between about 65%-75% methylation, HMGA1 comprises between about 80%-95% methylation, HOXB-AS3.2 comprises between about 60%-75% methylation, PPPIR18 comprises between about 10%-25% methylation, DNMT3A.2 comprises between about between about 90%-100% methylation, MLLT10 comprises between about between about 90%-100% methylation, DNMT3A.1 comprises between about 90%-100% methylation, PRKAG2 comprises between about 90%-100% methylation, TM4SF19 comprises between about 80%-100% methylation, CCDC9B comprises between about 85%-100% methylation, ZNF438 comprises between about 90%-100% methylation, MED13L comprises between about 90%-100% methylation, CHML comprises between about 90%-100% methylation, TULP4 comprises between about 90%-100% methylation, ZZEF comprises between about 90%-100% methylation, ACOT7 comprises between about 90%-100% methylation, LRPAP1 comprises between about 90%-100% methylation, PALM.2 comprises between about 90%-100% methylation, PALM.1 comprises between about 90%-100% methylation, ESRP2 comprises between about 80%-90% methylation, MEF2B comprises between about 75%-85% methylation, REC8 comprises between about 65%-80% methylation, PDYN-AS1 comprises between about 80%-90% methylation, GI MAP7 comprises between about 60%-85% methylation, XXYLT1 comprises between about 80%-90% methylation, HIVEP3 comprises between about 60%-75% methylation, WT1 comprises between about 70%-85% methylation, CD34.2 comprises between about 80%-95% methylation, CD34.1 comprises between about 80%-100% methylation, AIM2 comprises between about 50%-75% methylation, A4GALT comprises between about 65%-75% methylation, CTTN comprises between about 65%-85% methylation, CELF2 comprises between about 10%-40% methylation, HOXB-AS3.1 comprises between about 20%-30% methylation, MI RLET7BHG comprises between about 80%-90% methylation, HOXB3.2.2 comprises between about 75%-95% methylation, HOXB3.2.1 comprises between about 80%-90% methylation, and/or HOXB3.3 comprises between about 70%-85% methylation. It has been further contemplated that epitype 10 can comprise additional genes with a non-limiting range of methylation relative to methylation in a non-cancerous sample.

Regarding epitype 11, the following genes comprise a nonlimiting percentage range of methylation relative to methylation in a non-cancerous sample. ZSCAN25 comprises between about 80%-90% methylation, HCCA comprises between about 75%-85% methylation, RGS12 comprises between about 80%-90% methylation, HOXB3.1 comprises between about 60%-70% methylation, BEND7 comprises between about 60%-70% methylation, ALS2CL comprises between about 30%-80% methylation, HMGA1 comprises between about 70%-85% methylation, HOXB-AS3.2 comprises between about 15%-25% methylation, PPPIR18 comprises between about 5%-10% methylation, DNMT3A.2 comprises between about 90%-100% methylation, MLLT10 comprises between about 90%-100% methylation, DNMT3A.1 comprises between about 90%-100% methylation, PRKAG2 comprises between about 90%-100% methylation, TM4SF19 comprises between about 85%-95% methylation, CCDC9B comprises between about 80%-100% methylation, ZNF438 comprises between about 90%-100% methylation, MED13L comprises between about 90%-1005 methylation, CHML comprises between about 90%-100% methylation, TULP4 comprises between about 90%-100% methylation, ZZEF comprises between about 90%-100% methylation, ACOT7 comprises between about 90%-100% methylation, LRPAP1 comprises between about 90%-100% methylation, PALM.2 comprises between about 85%-100% methylation, PALM.1 comprises between about 80%-90% methylation, ESRP2 comprises between about 70%-80% methylation, MEF2B comprises between about 60%-80% methylation, REC8 comprises between about 40%-70% methylation, PDYN-AS1 comprises between about 40%-65% methylation, GI MAP7 comprises between about 10%-20% methylation, XXYLT1 comprises between about 35%-45% methylation, HIVEP3 comprises between about 20%-60% methylation, WT1 comprises between about 40%-50% methylation, CD34.2 comprises between about 15%-25% methylation, CD34.1 comprises between about 30%-40% methylation, AIM2 comprises between about 50%-90% methylation, A4GALT comprises between about 80%-95% methylation, CTTN comprises between about 80%-100% methylation, CELF2 comprises between about 90%-100% methylation, HOXB-AS3.1 comprises between about 65%-80% methylation, MI RLET7BHG comprises between about 90%-100% methylation, HOXB3.2.2 comprises between about 85%-100% methylation, HOXB3.2.1 comprises between about 90%-100% methylation, and/or HOXB3.3 comprises between about 80%-100% methylation. It has been further contemplated that epitype 11 can comprise additional genes with a non-limiting range of methylation relative to methylation in a non-cancerous sample.

Regarding epitype 12, the following genes comprise a nonlimiting percentage range of methylation relative to methylation in a non-cancerous sample. ZSCAN25 comprises between about 60%-75% methylation, HCCA comprises between about 40%-50% methylation, RGS12 comprises between about 55%-75% methylation, HOXB3.1 comprises between about 50%-65% methylation, BEND7 comprises between about 5%-10% methylation, ALS2CL comprises between about 10%-30% methylation, HMGA1 comprises between about 15%-20% methylation, HOXB-AS3.2 comprises between about 10%-20% methylation, PPPIR18 comprises between about 0%-10% methylation, DNMT3A.2 comprises between about between about 85%-100% methylation, MLLT10 comprises between about between about 85%-100% methylation, DNMT3A.1 comprises between about 75%-85% methylation, PRKAG2 comprises between about 90%-100% methylation, TM4SF19 comprises between about 80%-90% methylation, CCDC9B comprises between about 85%-100% methylation, ZNF438 comprises between about 85%-100% methylation, MED13L comprises between about 90%-100% methylation, CHML comprises between about 80%-90% methylation, TULP4 comprises between about 85%-95% methylation, ZZEF comprises between about 95%-100% methylation, ACOT7 comprises between about 90%-100% methylation, LRPAP1 comprises between about 85%-100% methylation, PALM.2 comprises between about 50%-70% methylation, PALM.1 comprises between about 50%-60% methylation, ESRP2 comprises between about 70%-80% methylation, MEF2B comprises between about 55%-65% methylation, REC8 comprises between about 35%-50% methylation, PDYN-AS1 comprises between about 60%-70% methylation, GI MAP7 comprises between about 5%-15% methylation, XXYLT1 comprises between about 15%-25% methylation, HIVEP3 comprises between about 35%-45% methylation, WT1 comprises between about 30%-50% methylation, CD34.2 comprises between about 5%-15% methylation, CD34.1 comprises between about 10%-20% methylation, AIM2 comprises between about 50%-85% methylation, A4GALT comprises between about 70%-80% methylation, CTTN comprises between about 55%-65% methylation, CELF2 comprises between about 85%-95% methylation, HOXB-AS3.1 comprises between about 60%-70% methylation, MI RLET7BHG comprises between about 80%-95% methylation, HOXB3.2.2 comprises between about 85%-100% methylation, HOXB3.2.1 comprises between about 85%-95% methylation, and/or HOXB3.3 comprises between about 75%-100% methylation. It has been further contemplated that epitype 12 can comprise additional genes with a non-limiting range of methylation relative to methylation in a non-cancerous sample.

Regarding epitype 13, the following genes comprise a nonlimiting percentage range of methylation relative to methylation in a non-cancerous sample. ZSCAN25 comprises between about 65%-75% methylation, HCCA comprises between about 50%-65% methylation, RGS12 comprises between about 60%-75% methylation, HOXB3.1 comprises between about 20%-35% methylation, BEND7 comprises between about 20%-35% methylation, ALS2CL comprises between about 5%-20% methylation, HMGA1 comprises between about 15%-25% methylation, HOXB-AS3.2 comprises between about 5%-10% methylation, PPPIR18 comprises between about 0%-10% methylation, DNMT3A.2 comprises between about between about 85%-100% methylation, MLLT10 comprises between about between about 85%-95% methylation, DNMT3A.1 comprises between about 75%-85% methylation, PRKAG2 comprises between about 90%-100% methylation, TM4SF19 comprises between about 85%-95% methylation, CCDC9B comprises between about 75%-85% methylation, ZNF438 comprises between about 85%-95% methylation, MED13L comprises between about 90%-100% methylation, CHML comprises between about 75%-85% methylation, TULP4 comprises between about 85%-95% methylation, ZZEF comprises between about 90%-100% methylation, ACOT7 comprises between about 90%-100% methylation, LRPAP1 comprises between about 80%-100% methylation, PALM.2 comprises between about 60%-70% methylation, PALM.1 comprises between about 50%-65% methylation, ESRP2 comprises between about 15%-45% methylation, MEF2B comprises between about 10%-25% methylation, REC8 comprises between about 10%-20% methylation, PDYN-AS1 comprises between about 15%-30% methylation, GI MAP7 comprises between about 5%-15% methylation, XXYLT1 comprises between about 15%-25% methylation, HIVEP3 comprises between about 15%-30% methylation, WT1 comprises between about 10%-20% methylation, CD34.2 comprises between about 10%-20% methylation, CD34.1 comprises between about 20%-30% methylation, AIM2 comprises between about 55%-80% methylation, A4GALT comprises between about 70%-85% methylation, CTTN comprises between about 70%-90% methylation, CELF2 comprises between about 75%-85% methylation, HOXB-AS3.1 comprises between about 40%-50% methylation, MI RLET7BHG comprises between about 75%-85% methylation, HOXB3.2.2 comprises between about 80%-100% methylation, HOXB3.2.1 comprises between about 80%-95% methylation, and/or HOXB3.3 comprises between about 75%-95% methylation. It has been further contemplated that epitype 13 can comprise additional genes with a non-limiting range of methylation relative to methylation in a non-cancerous sample.

In some embodiments, the epitypes of any preceding aspect are determined from epigenetic markers. In some embodiments, the epitypes of any preceding aspect are determined from genetic markers. In some embodiments, the epitypes of any preceding aspect are determined from any combination of epigenetic markers and genetic markers. For example, one or more genetic markers listed in Table 2 can be used alone or in combination with epigenetic markers to determine the epitype, or can be used along with the epitype to determine the likelihood of having or developing the diseases and/or disorders disclosed herein.

In some embodiments, the epitype of any preceding aspect is associated with a genetic aberration. As used herein, a “genetic aberration” refers to an alteration or change to a DNA sequence, wherein the gene encodes a defective gene product, a normal gene product, or no longer produces a gene product. A genetic aberration includes, but is not limited to a gene mutation, a gene fusion event, a chromosomal aberration, a gene translocation, a gene deletion, a gene duplication, or a gene inversion. In some embodiments, the genetic aberration occurs at one or more of the following genes including, but not limited to ASXL1, BCOR, BRAF, CBL, DNMT3A, ETV6, EZH2, FBXW7, GATA2, IDH1, IKZF1, JAK2, KIT, KRAS, MLL, MPL, NF1, NPM1, NRAS, PHF6, PTEN, PTPN11, RAD21, RUNX1, SF1, SF3A1, SF3B1, SFRS2, STAG2, TET2, TP53, U2AF1, WT1, ZRSR2, CEBPA-sm, CEBPA-dm, FLT3-ITD, FLT3-TKD, IDH2p172, IDH2p140, inv(3)/t(3;3), t(9:22), Monosomy 5, del (5q), Monosomy 7, del (7q), Abnormal chr. 7 (other), Plus8, +8q, del (9q), Abnormal chr. 12, Plus 13, Monosomy 17, abnormal chr. 17p, Monosomy 18, del (18q), Monosomy 20, del (20q), Plus 21, Plus 22, Minus Y, t(8,21), inv(16), t(6;9), Plus 11, +11q, Abnormal chr. 4, Complex karyotype, t(9;11), or t(v; 11)(other).

In some embodiments, the epitypes are further divided into superclusters (SC). In some embodiments, the epitypes are further divided into 2 or more SC. In some embodiments, the epitypes are further divided into 2, 3, 4, 5, 6, 7, 8, 9, 10, 11, 12, 13, 14, 15, 16, 17, 18, 19, 20, or more SC. In some embodiments, the epitypes are further divided into 4 SC.

In some embodiments, the SC comprises epitypes enriched for alterations to genes encoding at least one transcription factor. In some embodiments, said alterations include, but are not limited to a gene fusion event and a gene mutation. In some embodiments, the gene fusion event includes, but is not limited to a PML-RARA gene fusion, an inv(16)/CBFB gene fusion, or an AML-ETO gene fusion. In some embodiments, the gene mutation includes, but is not limited to a CEBPA gene mutation. In some embodiments, the gene fusion event or the gene mutation results in arresting myeloid development.

In some embodiments, the SC comprises epitypes enriched for chromosomal rearrangements generating gene fusion events. In some embodiments, In some embodiments, the chromosomal rearrangements include, but are not limited to rearrangements to the KMT2A (MLL) genes on chromosome 11q23. It should be noted that KMT2A can comprise multiple gene fusion partners in AML, which is described by Winters and Bernt (Winters and Bernt. “MLL-Rearranged Leukemias—An update on Science and Clinical Approaches”. Front. Pediatr. 9 Feb. 2017), which is incorporated herein in its entirety for its teachings of the fusion partners of KMT2A (MLL).

In some embodiments, the SC comprises epitypes enriched in NPM1 gene mutations. In some embodiments, the NPM1 gene mutations occur alone or in combination with other genes, including but not limited to DNMT3A, TET2, IDH1, and/or IDH2. In some embodiments, the epitypes are enriched for gene mutations to DNMT3A, TET2, IDH1, and/or IDH2, but lacking a mutation to NPM1.

In some embodiments, the SC comprises epitypes that lack a mutation pattern, but retain gene mutations associated with genomic instability. In some embodiments, the gene mutations associated with genomic instability includes, but are not limited TP53 mutations and/or complex karyotypes.

In some embodiments, the SC comprises epitypes that display stem cell-like traits and/or characteristics.

In some embodiments, the thirteen epitypes are further divided into 4 SC selected from a transcription factor (TF)-SC, an MLL-SC, a NPM1-SC, or a stem-cell like (SL)-SC.

In some embodiments, the TF-SC includes, but is not limited to epitype 1, epitype 2, epitype 3, and epitype 4. In some embodiments, the TF-SC comprises a disruption and/or mutation to one or more transcription factors (TFs). In some embodiments, the MLL-SC includes, but is not limited to epitype 5 and epitype 6. In some embodiments, the MLL-SC comprises a rearrangement, mutation, and/or translocation of a KMT2A/MLL gene. In some embodiments, the NPM1-SC includes, but is not limited to epitype 7, epitype 8, epitype 9, and epitype 10. In some embodiments, the NPM1-SC comprises at least one NPM1 mutation. In some embodiments, the SL-SC includes, but is not limited to epitype 11, epitype 12, and epitype 13. In some embodiments, the SL-SC displays DNA methylation patterns similar to DNA methylation patterns in hematopoietic stem cells.

Proinflammatory signaling is commonly associated with cancer and is often generated by mutations in tumor cells. In AML, a gain-of-function FLT-internal tandem duplication (FLT-ITD) mutation activates the Janus kinases/signal transducer and activator of transcription (JAK/STAT) pathway and are associated with poor outcomes. Herein, cancers comprising an FLT-ITD mutation further comprise hypomethylation (or decreased methylation) of a signal transducer and activator of transcription (STAT) gene leading to activation of the JAK/STAT pathway. In some embodiments, a cancerous tissue or sample with the FLT-ITD mutation comprises at least a 15% decrease in methylation at the STAT gene relative to a non-cancerous tissue or sample. In some embodiments, a cancerous tissue or sample with the FLT-ITD mutation comprises 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% decreased methylation at the STAT gene relative to a non-cancerous tissue or sample. In some embodiments, the cancerous tissue or sample with the FLY-ITD mutation comprises 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, or 85% methylation relative to a non-cancerous tissue or sample. In some embodiments, the STAT gene comprises STAT1, STAT2, STAT3, STAT4, STAT5A, STAT5B, and/or STAT6. In some embodiments, the hypomethylation (or decreased methylation) occurs at STAT1, STAT2, STAT3, STAT4, STAT5A, STAT5B, STAT6, or combinations thereof.

FLT-ITD mutations were found to be spread across several epitypes. Thus, in some embodiments, the epitype of any preceding aspect is further associated with an FLT-ITD mutation. In some embodiments, the NPM/gene mutation and the FLT-ITD mutation occur within an epitype. In some embodiments, the chromosomal rearrangement(s) and the FLA-ITD mutation occur within an epitype. In some embodiments, the KMT2A (MLL) rearrangement(s) and the FLT-ITD mutation occur within an epitype. In some embodiments, alterations, including but not limited to gene fusion events or gene mutations, to genes encoding at least one transcription and the FLT-ITD mutation occur within an epitype. In some embodiments, the PML-RARA gene fusion and the FLT-ITD mutation occur within an epitype. In some embodiments, the inv(16)/CBFB gene fusion and the FLT-ITD mutation occur within an epitype. In some embodiments, the AML-ETO gene fusion and the FLT-ITD mutation occur within an epitype. In some embodiments, the DNMT3A mutation and the FLT-ITD mutation occur within an epitype. In some embodiments, the TET2 mutation and the FLT-ITD mutation occur within an epitype. In some embodiments, the IDH1 mutation and the FLT-ITD mutation occur within an epitype. In some embodiments, the IDH2 mutation and the FLT-ITD mutation occur within an epitype. In some embodiments, the TP53 mutation and the FLT-ITD mutation occur within an epitype. In some embodiments, the epitype comprises the FLT-ITD mutation.

It should also be noted that cancer patients, including but not limited to AML patients, can relapse after achieving partial or complete remission. As used herein, the term “relapse” refers to the return or reappearance of cancer cells, or display of signs or symptoms of cancer after a period of improvement. Thus, a patient can be categorized into one epitype during the first appearance or signs of cancer, but then be categorized into the same or different epitype after relapse. One non-limiting example includes a patient being categorized into epitype 1 during the first appearance or signs of cancer, but then is categorized into epitype 2 after relapse.

### Kits for Detecting an Epigenetic Pattern

In one aspect, disclosed herein is a kit for detecting an epigenetic modification of a deoxyribonucleic acid (DNA) sequence from a tissue sample. In some embodiments, the tissue sample is derived from a subject.

In some embodiments, the tissue sample comprises a blood sample. In some embodiments, the tissue sample comprises a tissue biopsy. In some embodiments, the tissue sample comprises a urine sample. In some embodiments, the tissue sample comprises a fecal sample.

In some embodiments, the kit comprises a DNA denaturing reagent. In some embodiments, the DNA denaturing reagent comprises a salt, a basic compound, or a chemical compound. In some embodiments, the DNA denaturing reagent comprises sodium hydroxide.

In some embodiments, the DNA denaturing reagent comprises dimethyl sulfoxide (DMSO). In some embodiments, the kit does not comprise a DNA denaturing reagent. In some embodiments, the kit requires heat to denature the DNA.

In some embodiments, the kit comprises a DNA conversion reagent. In some embodiments, the DNA conversion reagent comprises bisulfite compound. In some embodiments, the DNA conversion reagent comprises sodium bisulfite. In some embodiments, the DNA conversion reagent converts cytosine to thymine.

In some embodiments, the kit comprises a binding buffer, a washing buffer, and an elution buffer. In some embodiments, the binding buffer comprises any combination of the following reagents selected from guanidine hydrochloride, guanidine thiocyanate, isopropanol, sodium chloride, or a buffered solution (including, but not limited to 3-(N-morpholino) propanesulfonic acid (MOPS), 4-(2-hydroxyethyl)-1-piperazineethanesulfonic acid (HEPES), phosphate buffered saline (PBS), tris buffered saline (TBS), Tris-HCl, and Tris-Acetate). In some embodiments, the washing buffer comprises any combination of the following reagents selected from ethanol, sodium chloride, or a buffered solution (including, but not limited to MOPS, HEPES, PBS, TBS, Tris-HCl, and Tris-Acetate). In some embodiments, the elution buffer comprises any combination of the following reagents selected from EDTA, ammonium acetate, magnesium acetate, imidazole, sodium chloride, sodium phosphate, or a buffered solution (including, but not limited to MOPS, HEPES, PBS, TBS, Tris-HCl, and Tris-Acetate).

In some embodiments, the epigenetic pattern comprises a methylation of a deoxyribonucleic acid (DNA) sequence. In some embodiments, the methylation occurs at a cytosine nucleotide. In some embodiments, the methylation occurs at a cytosine-phosphate-guanosine (CpG) island of the nucleic acid. In some embodiments, the methylation occurs at an adenosine nucleotide.

In some embodiments, the methylation modification on the DNA molecule is further sequenced by methylation iPLEX (Me-iPLEX) technology. In some embodiments, the methylation modification of the DNA molecule is further sequenced by restrictive enzyme-based sequencing approaches. In some embodiments, the methylation modification of the DNA molecule is further sequenced by affinity enrichment-based sequencing approaches. In some embodiments, the methylation modification of the DNA molecule is further sequenced by bisulfite conversion-based sequencing approaches. In some embodiments, the methylation modification of the DNA molecule is further sequenced by DNA hydroxymethylation sequencing approaches.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Epigenetic Phenocopying Expands Molecular Risk Assessment in Acute Myeloid Leukemia (AML)

### Introduction

Genetic profiling in acute myeloid leukemia (AML) forms the basis for both initial treatment selection and also need for aggressive allogeneic stem cell transplant. To improve this, genome-wide epigenetic signatures have been described that underlie biological features of AML cells and their utility to classify patients. Herein, it is determined whether DNA methylation can add to genetic profiling and other known markers to better assign treatment of AML patients.

### Results

### Targeted, High-Throughput Analysis of AML Epitype Using Methylation-iPLEX

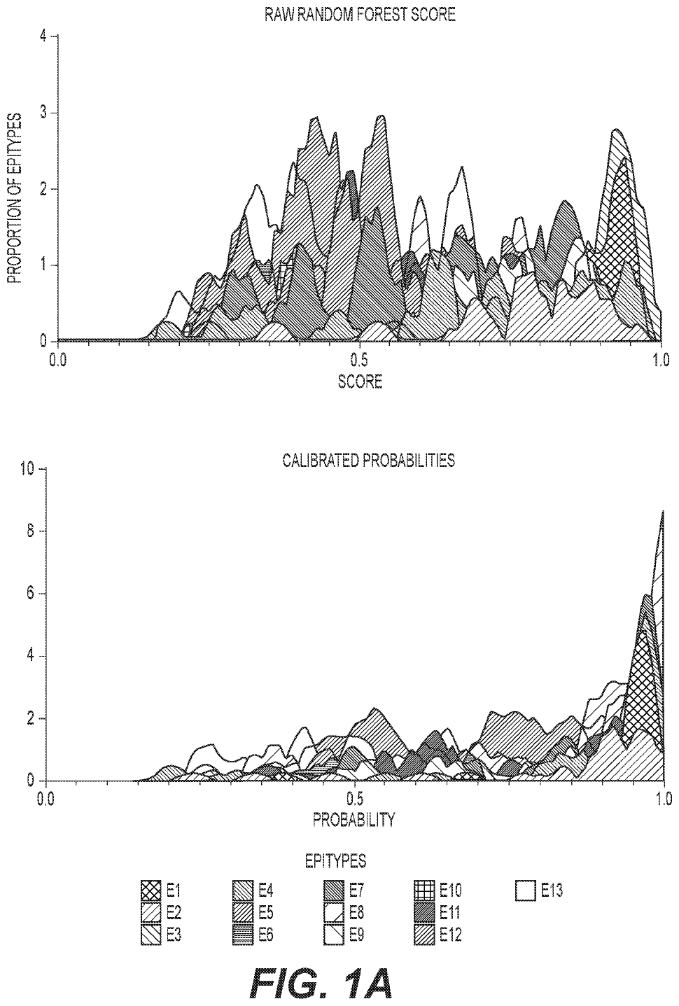

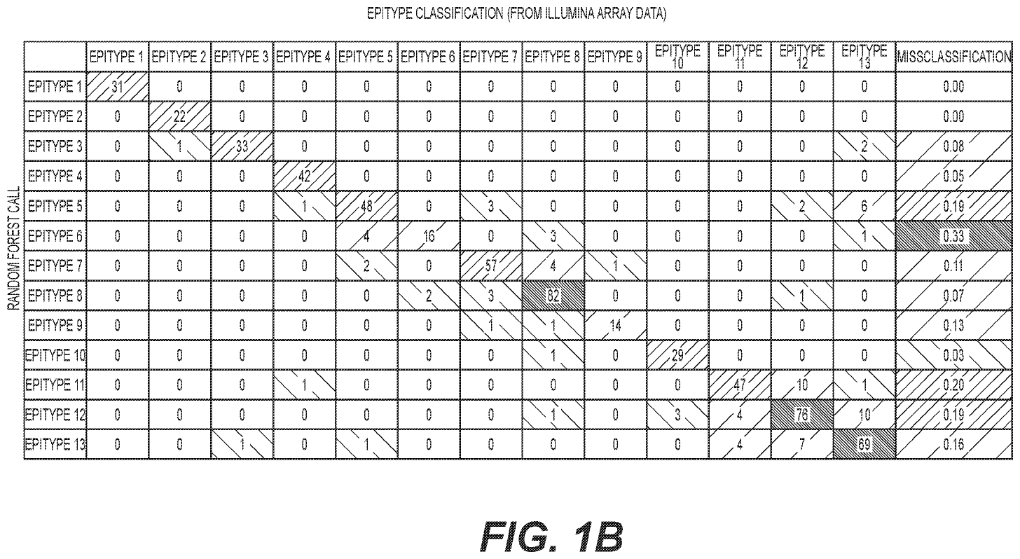

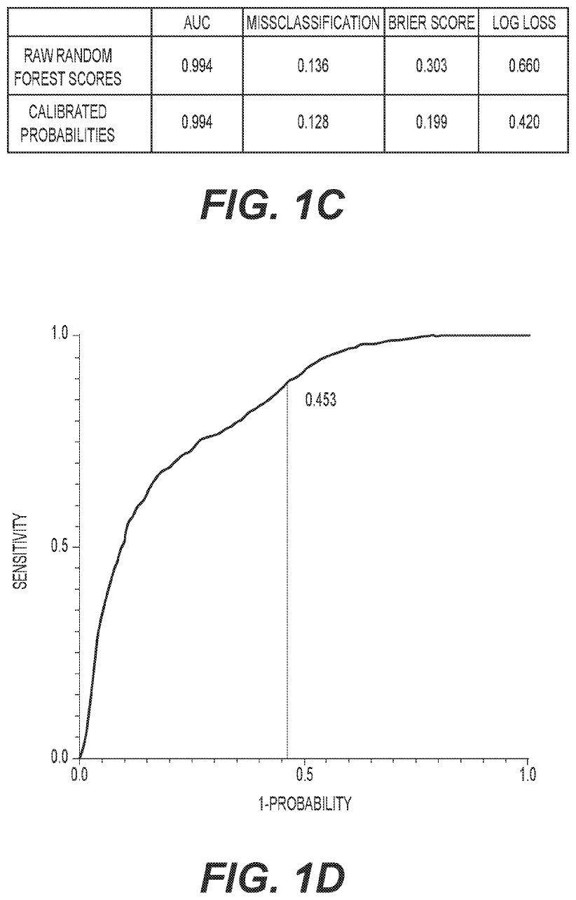

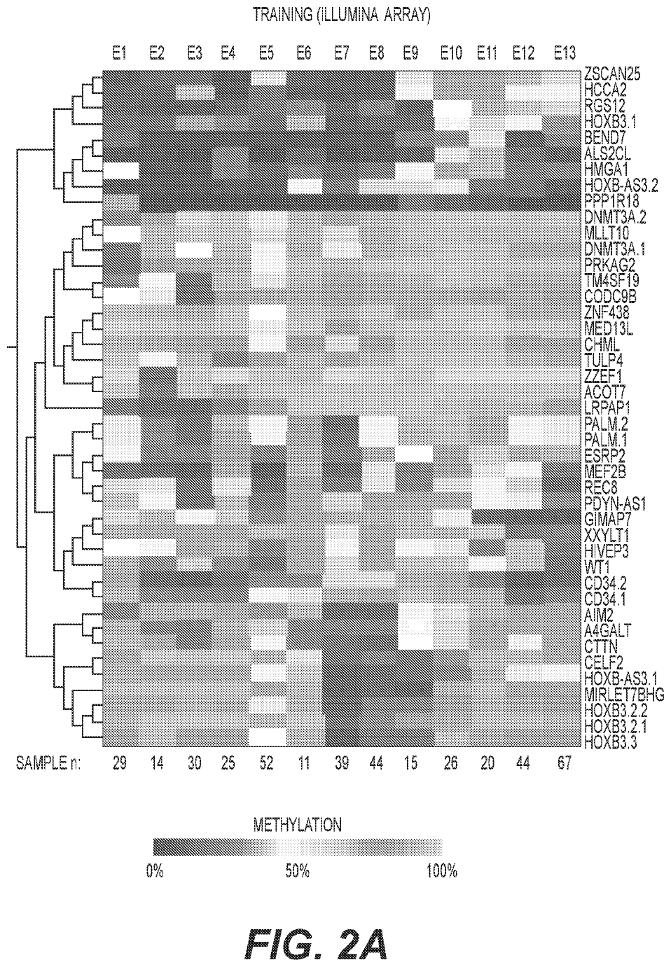

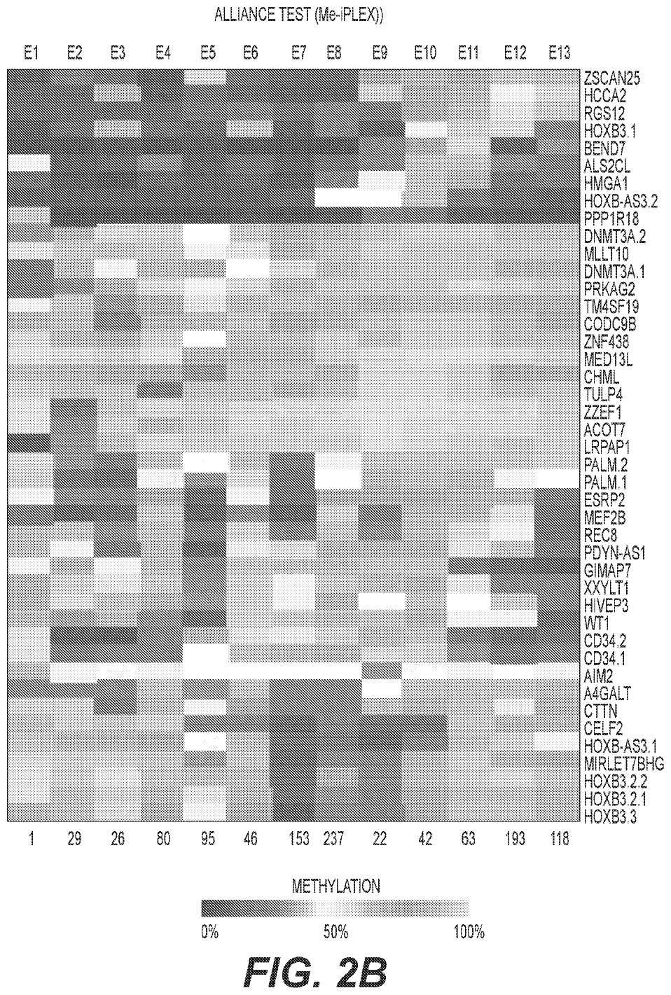

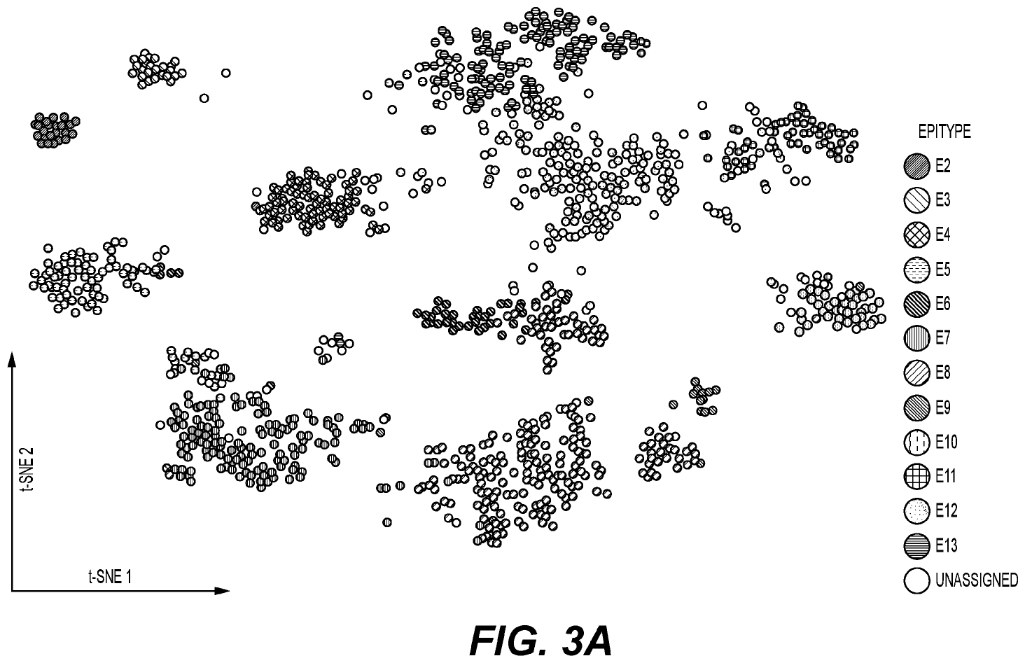

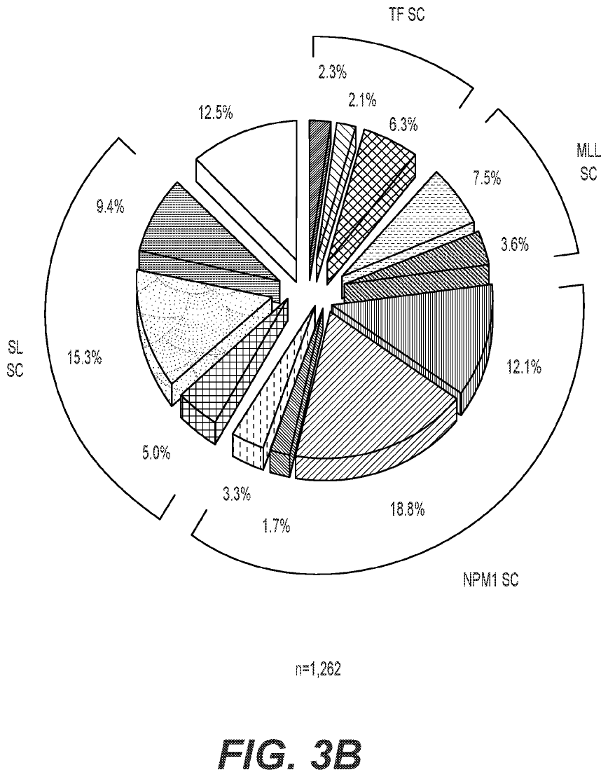

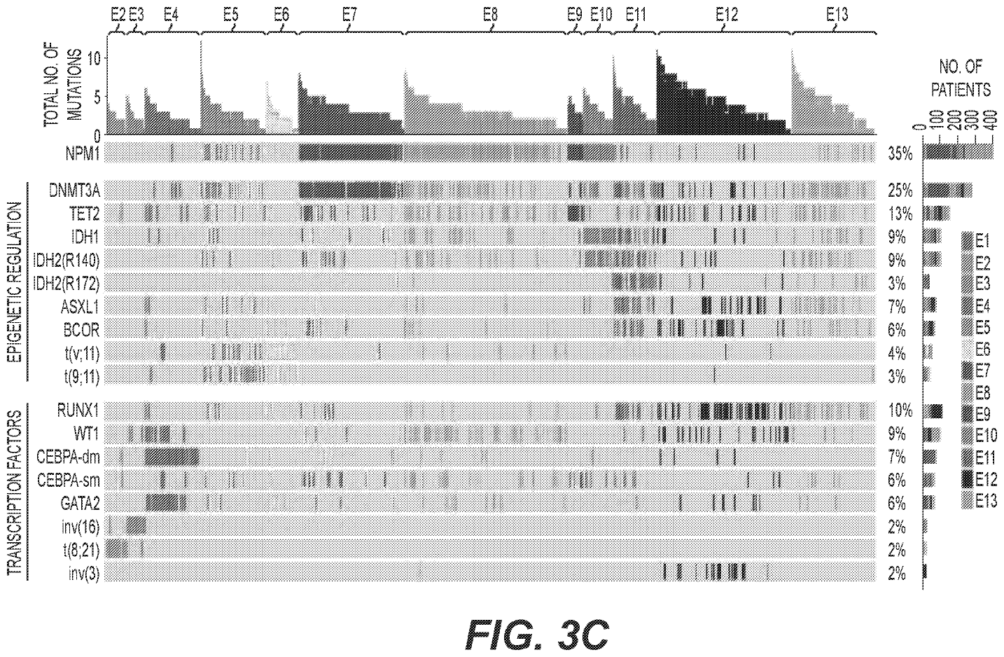

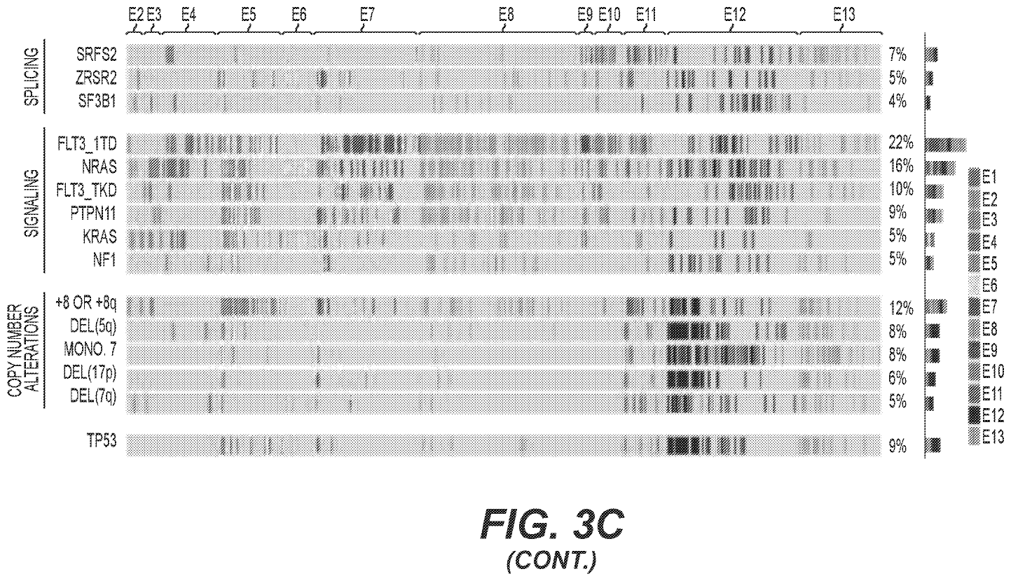

A method using MassARRAY technology that accurately quantifies DNA methylation levels of single CpGs of interest in a multiplexed, high-throughput approach termed methylation-iPLEX (Me-iPLEX) was once described. To develop a Me-iPLEX assay capable of accurately assigning AML patients into one of the 13 epitypes, a panel of 43 CpGs were identified that recapitulated the epitype classification defined from a prior genome-wide study with >90% accuracy (see Methods and FIGS. 1 and 2). The majority of these CpGs were located within or proximal to genes lacking known associations to AML, however a subset was AML related, including WT1, DNMT3A, MLLT10, MEF2B, CD34, and HOXB-AS3 (TABLE 1). A cohort of 1,262 AML patients enrolled on studies conducted by the Alliance for Clinical Trials in Oncology were assayed, assigning a unique epitype in 1,105 patients (87.5%). Visualization of methylation patterns of all samples by t-SNE revealed a high degree of separation between most epitypes in a similar arrangement as found previously, with the majority of unassigned patients clustering on the periphery of known epitypes (FIG. 3A). Thirteen epitypes were grouped into 4 higher-order ‘superclusters’ (SCs) based on similarity of DNA methylation patterns and other biological features. These included the transcription factor (TF)-SC, which incorporates epitypes E1-4 that involve disruption of TFs involved in myeloid development; the MLL-SC, which are enriched in KMT2A/MLL rearrangements (E5,6); the NPM1-SC, which display a high frequency of NPM/mutations (E7-10); and the stem cell-like (SL)-SC, which display developmental DNA methylation states similar to hematopoietic stem cells (E11-13) (FIG. 3B).

### Genetic Composition of Epitypes Reveals Epiphenocopying of Genomic Aberrations

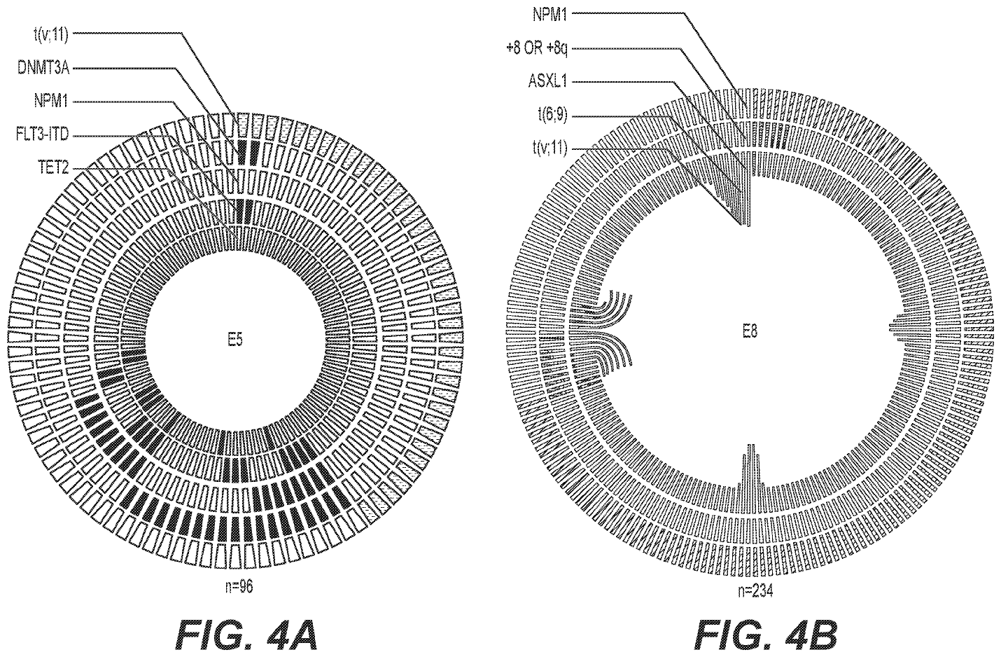

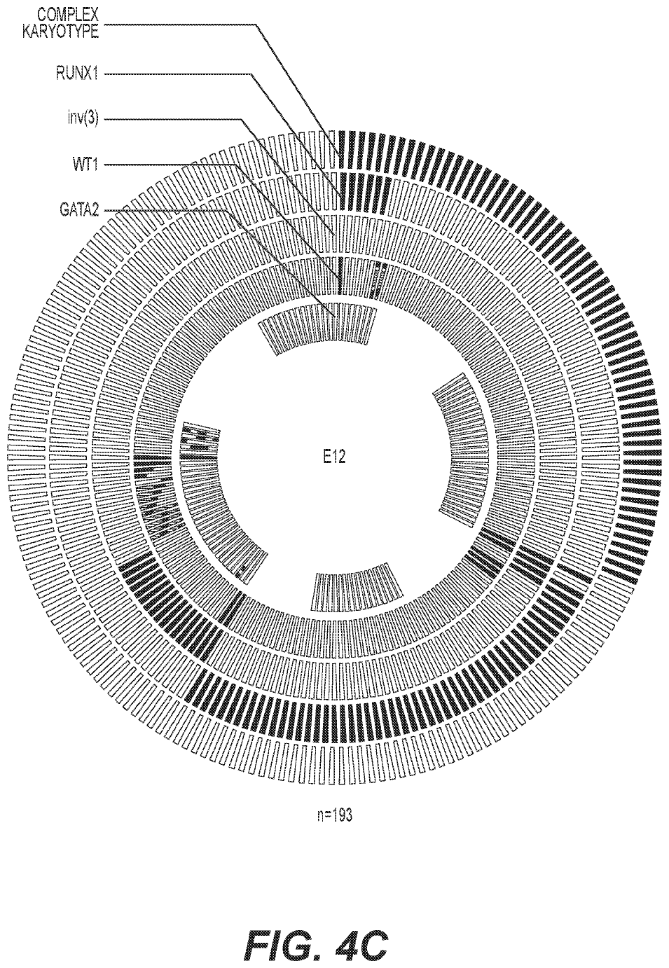

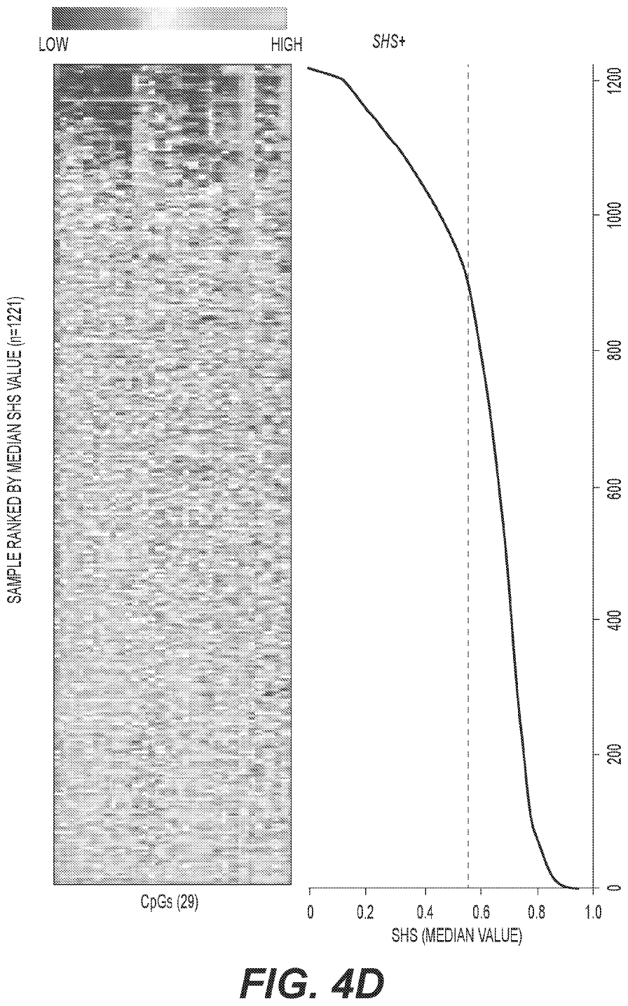

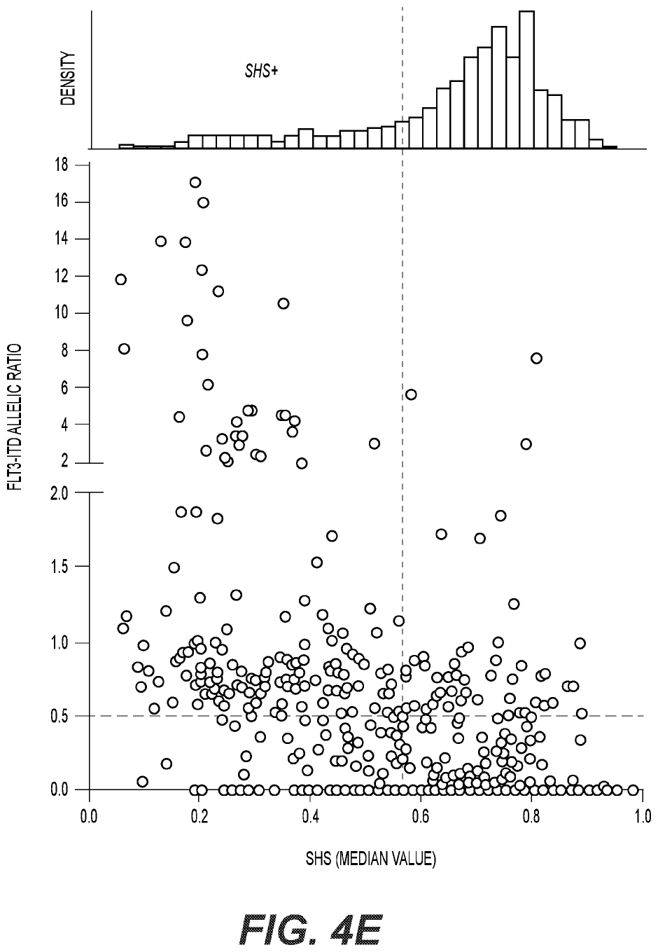

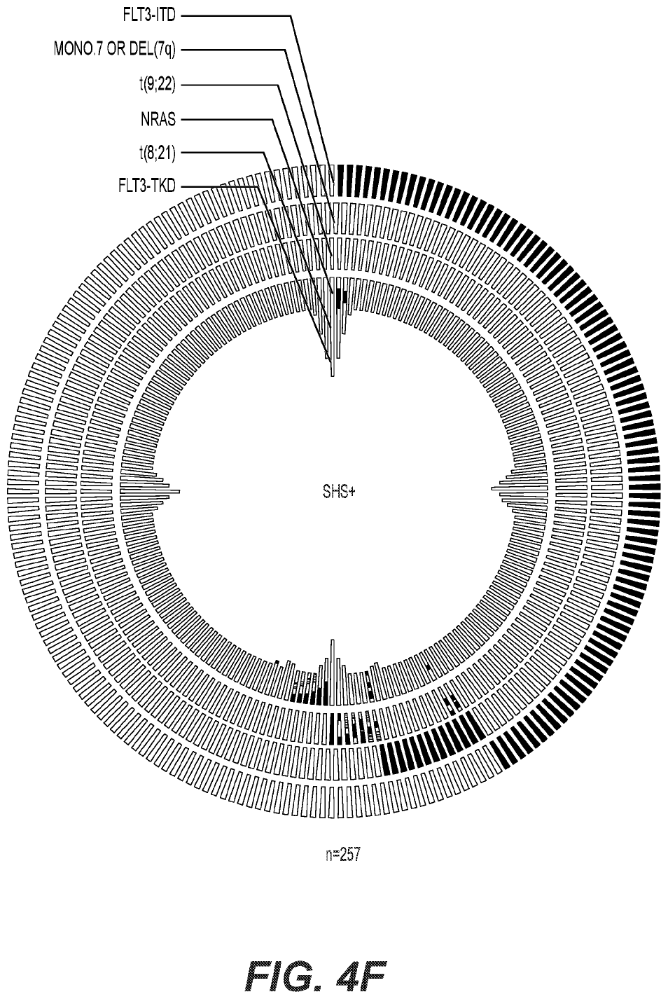

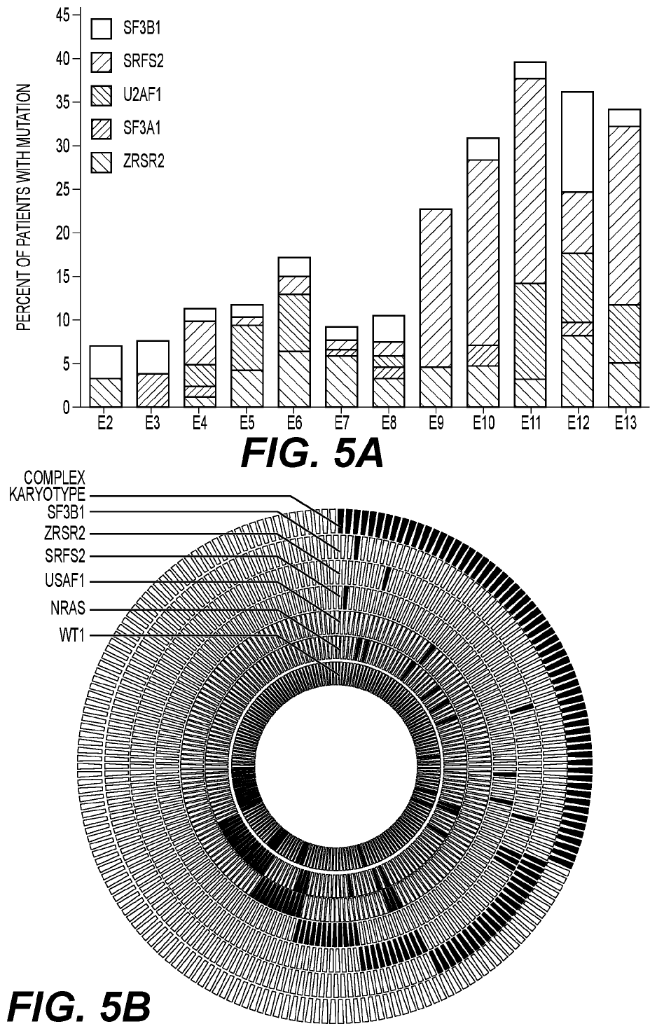

A study identified an association between epitypes and recurrent mutations in AML, however lacked sufficient depth to fully investigate their underlying genetic composition. Investigation of genetic aberrations revealed that the majority of epitypes are associated with a dominant mutation. It was found that t(8;21), inv(16), and CEBPA-dm were strongly associated with E2, E3, and E4 (present in 73%, 88% and 85% of patients), respectively (FIG. 3C, TABLE 2). NPM1 mutations were present in 376/467 (81%) of patients within epitypes E7-10 (NPM1-SC), with co-association of DNMT3A, TET2, and IDH1/2 mutations in 82%, 77% and 98% of E7, E9 and E10, respectively. Double mutations in TET2 were found at approximately twice the rate in E9 than other epitypes (59% vs. 31%, P<0.05). In the MLL-SC epitypes, rearrangements involving KMT2A (MLL) involved 42% and 70% of E5 and E6, respectively. E11 was composed of 86% of patients with IDH1/2, however lacked NPM1 co-mutations contrary to E10. All IDH2-R172 mutations occurred in E11. Lastly, E12 and E13 were not associated with a dominant mutation in the majority of samples (despite showing the highest overall number of mutations per epitype), with DNMT3A occurring in 35% of E13. Next, patients that were assigned to a particular epitype yet lacked the respective dominant mutation, a phenomenon we termed ‘epiphenocopying’, were investigated. E5 and E8 as these epitypes were the focus of the study and retained sufficient epiphenocopies for analysis. Non-KMT24-rearranged E5 patients (60%) were found to be significantly enriched for DNMT3A, NPM1, and FLT3-ITD mutations comprising approximately half of epiphenocopies (FIG. 4A). For E8, it was found that the 71/234 (30%) of patients lacking NPM1 mutations were enriched in ASXL1 mutations and gains of chromosome 8q, and also contained all t(6;9) rearrangements (FIG. 4B). E12 was enriched for patients displaying complex karyotype (CK) in approximately one-third (31.6%) of patients, typically displaying del (17p), del (7q) and/or del (5q), and TP53 mutations (FIG. 4C). It was also found that aberrations in inv(3), RUNX1, WT1 and GATA2 mutations were mutually exclusive of CK in E12 (FIG. 4C). Mutations in spliceosome genes were also enriched in E12, with highly prevalent SF3B1 mutations also mutually exclusive of CK along with other spliceosome components (FIGS. 5A and 5B). The E12 mutational spectrum was uniquely reminiscent of myelodysplastic syndrome (MDS) among epitypes (despite exclusion of patients with identified antecedent MDS in the cohort) and indicates that the constellation of mutations along with CK converge in E12 demonstrating a common underlying biological function of these genetic lesions in AML.

### The STAT Hypomethylation Signature (SHS) Identifies Epiphenocopies of FLT3-ITD

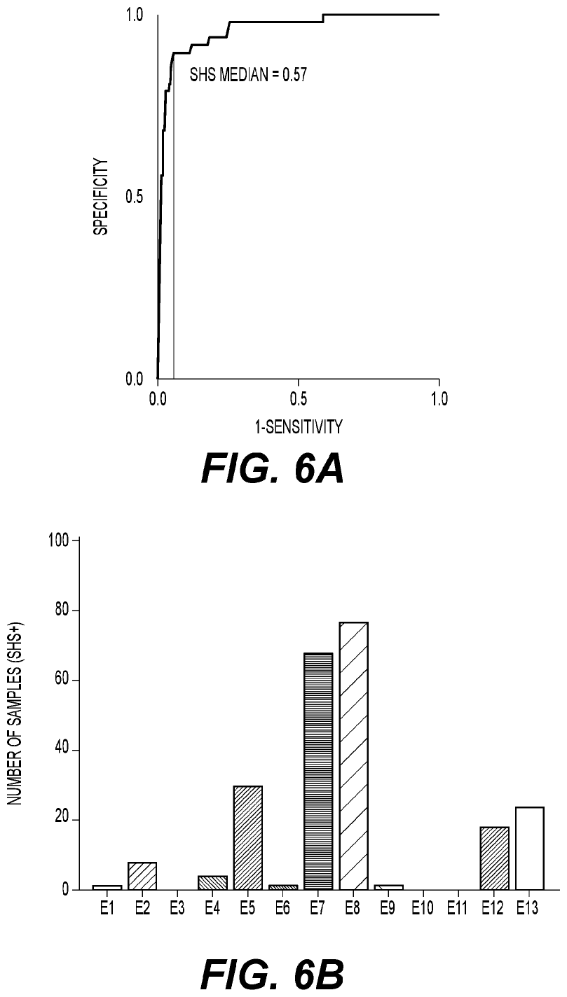

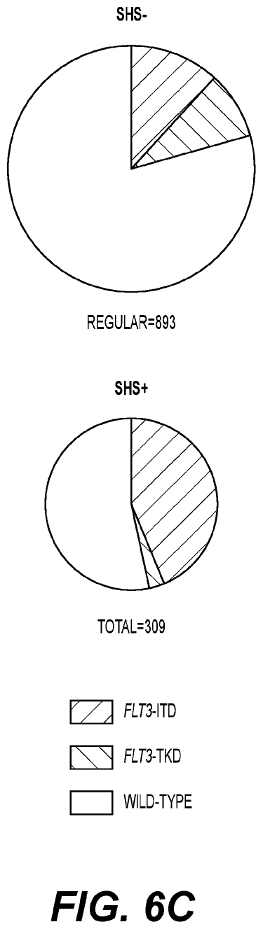

SHS is associated with FLT3-ITD, one of the most frequent genomic markers known to worsen outcome in AML. A novel Me-iPLEX panel was developed to determine SHS status. SHS was measured in 1,221/1,262 patients separating SHS-negative (high methylation) and SHS-positive (lower methylation) groups, the latter comprising 21% of patients (FIG. 4D). SHS-positivity was primarily observed in E5, E7, E8, E11 and E12 (FIG. 6B). A general inverse relationship of FLT3-ITD allelic ratio with SHS median value was found (FIG. 4E). However, some samples were discordant between FLT3-ITD (allelic ratio>0.5) and SHS+, with 11% of SHS-negative patients exhibiting FLT3-ITD+ and 53% of SHS+ patients lacking FLT3-ITD (FIG. 6C). It was found that SHS+ patients that lacked FLT3-ITD (epiphenocopies) were significantly enriched for monosomy 7/del (7q), t(9;22), t(8;21), FLT3-TKD and NRAS mutations versus FLT3-ITD+ patients (FIG. 4F). These results show that these genetic events involve aberrant STAT pathway activation in a similar manner to FLT3-ITD.

### Impact of the DNA Methylation Signatures on Clinical Outcomes in AML

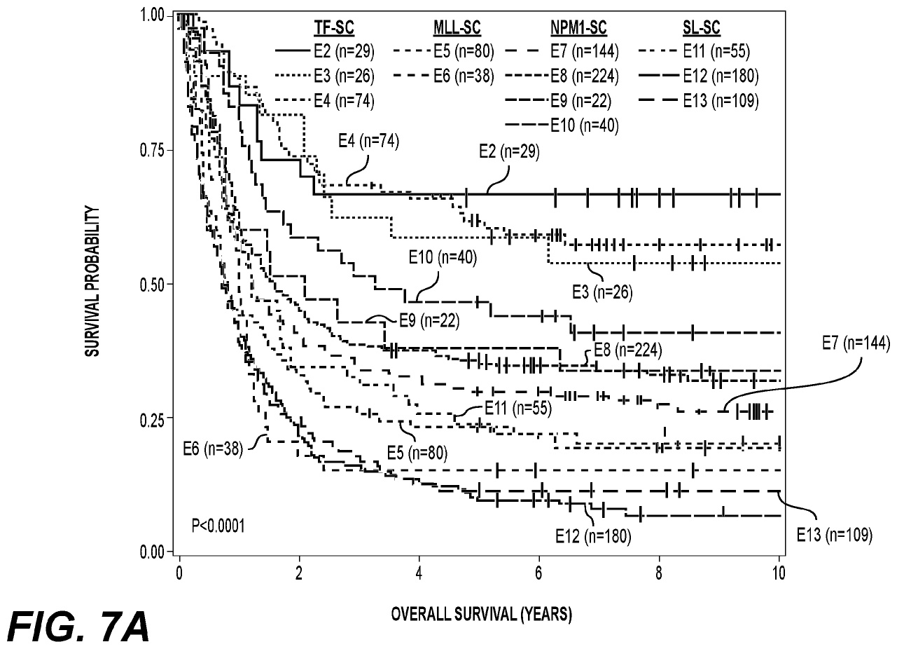

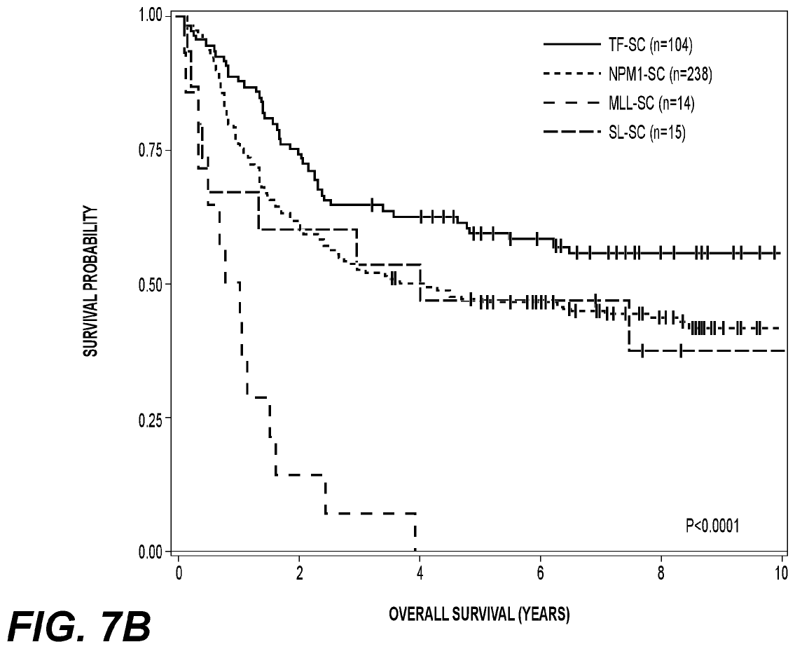

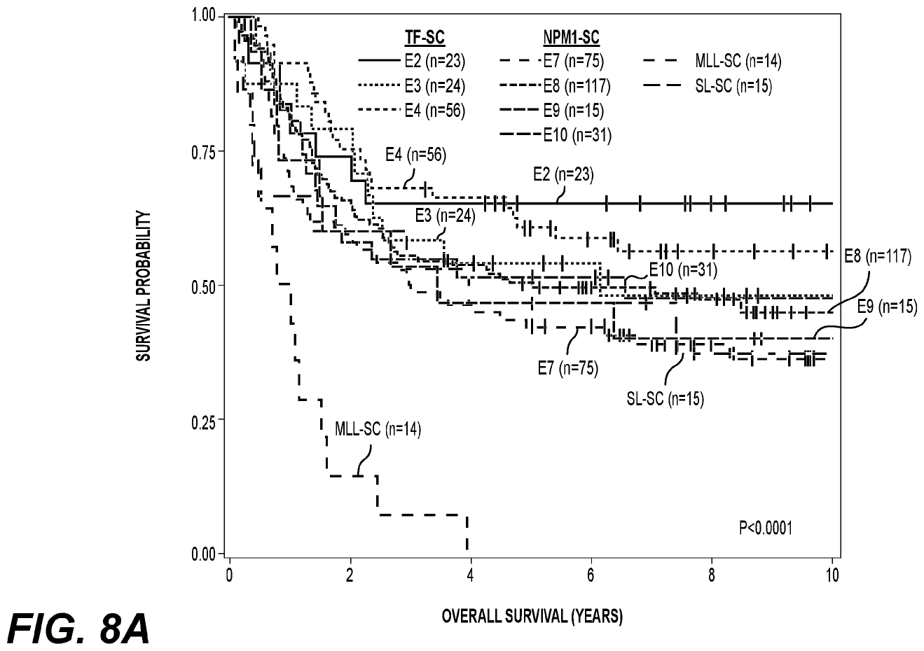

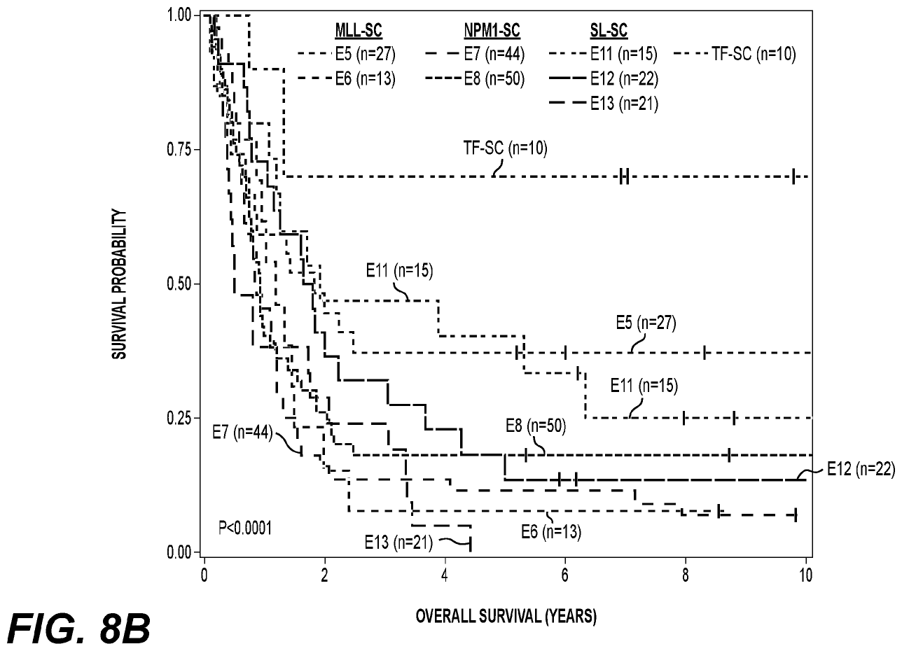

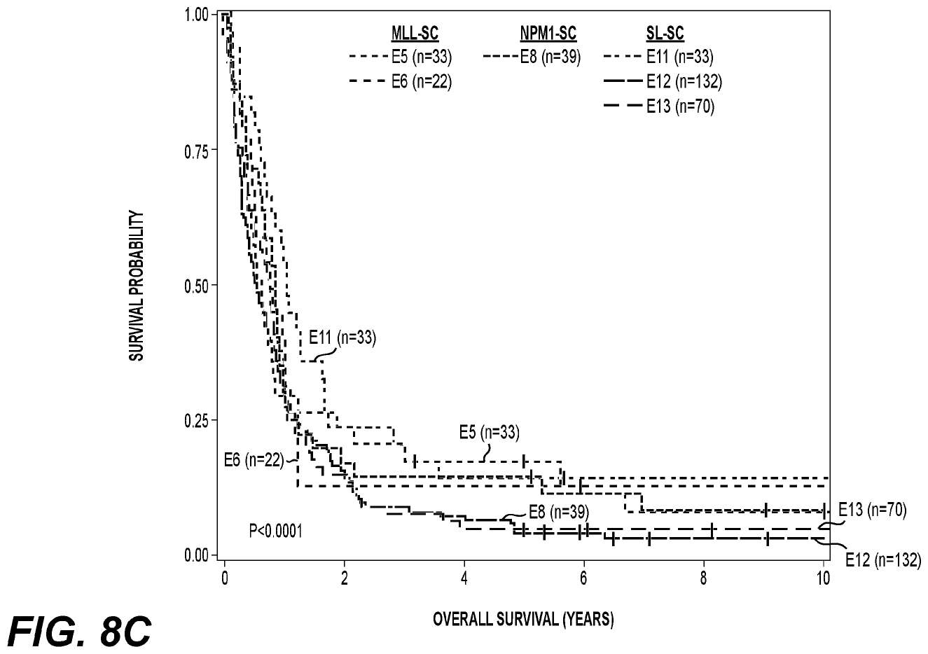

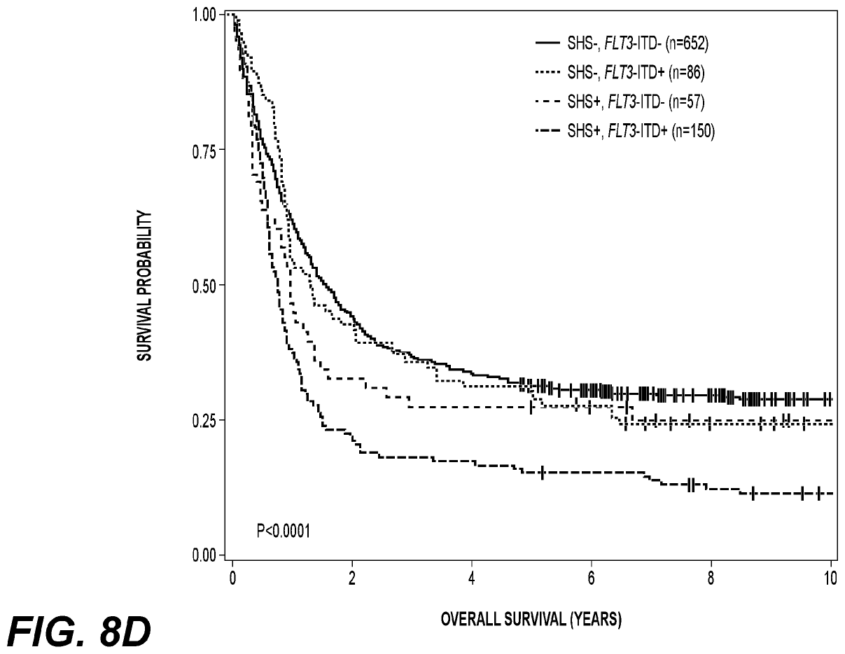

Next, patients with available clinical annotation that were assigned to both a unique epitype and SHS classification (1,021 patients) to examine associations between DNA methylation signatures with demographic, clinical features and outcome were investigated. Patients received similar cytarabine/daunorubicin-based treatment regimens and none underwent allo-HCT in first remission per protocol. Epitypes displayed significant differences between multiple pre-treatment demographic features and hematological parameters (TABLE 3). Epitypes delineated broad differences in clinical outcomes, such as complete remission and relapse, as well as disease-free survival and overall survival (OS) (TABLE 4). In line with the major associated genetic aberrations, epitypes belonging to the TF-SC (E2-4) and NPM1-SC (E7-10) generally displayed favorable and intermediate outcomes, respectively, whereas epitypes E5,6 and E11-13 (MILL- and SC-SCs, respectively) were associated with adverse outcomes (FIG. 7A). To compare epitype with genetic features, OS was assessed by epitype within the ELN risk groups. Epitypes containing fewer patients were grouped by SC within ELN groups. In the ELN favorable group, patients were observed belonging to the MLL-SC displayed less favorable outcome, despite exclusion of KMT2A rearrangements in this risk category (FIGS. 7B and 8A, P<0.0001). Furthermore, it was observed that within the ELN intermediate risk classification, patients belonging to the TF-SC were associated with a more favorable risk despite lacking favorable risk genetic markers (FIG. 8B). Within the ELN adverse risk group, E12,13 performed worse (FIG. 8C). Comparing SHS with FLT3-ITD, we observed that inferior survival associated with FL73-ITD was negated in SHS-negative patients, and SHS-positivity portended poorer survival in FLT3-ITD-negative patients (P<0.0001; FIG. 8D).

### Assessment of the Integrated Impact of DNA Methylation Using Machine Learning

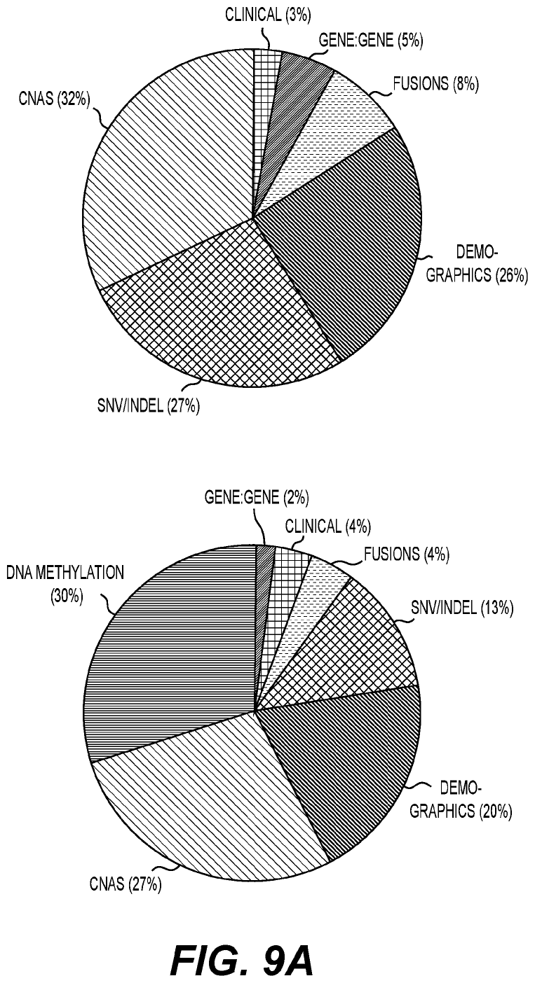

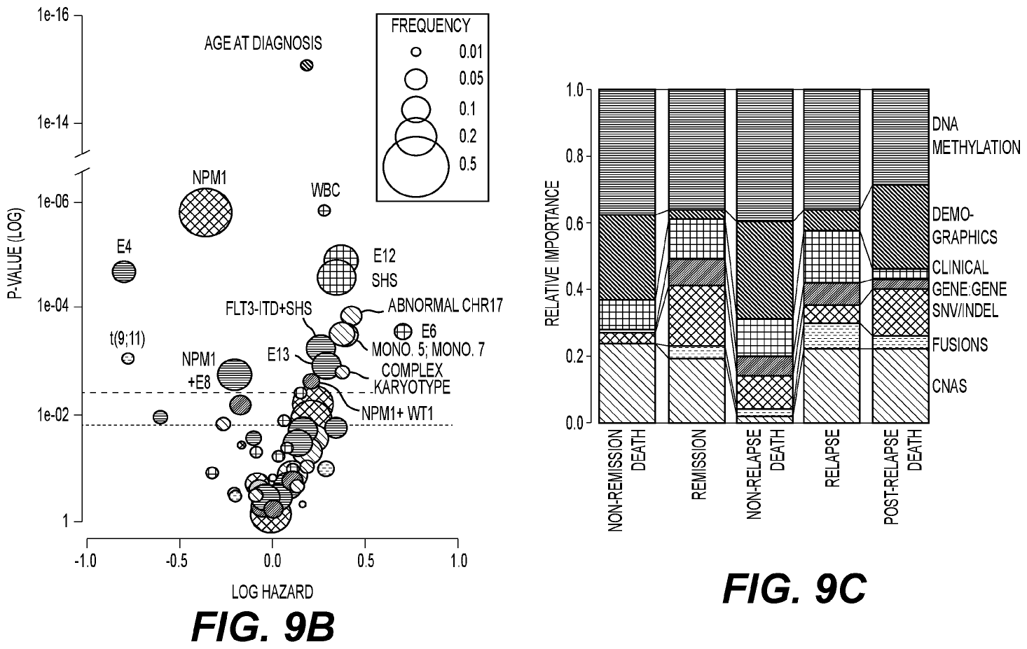

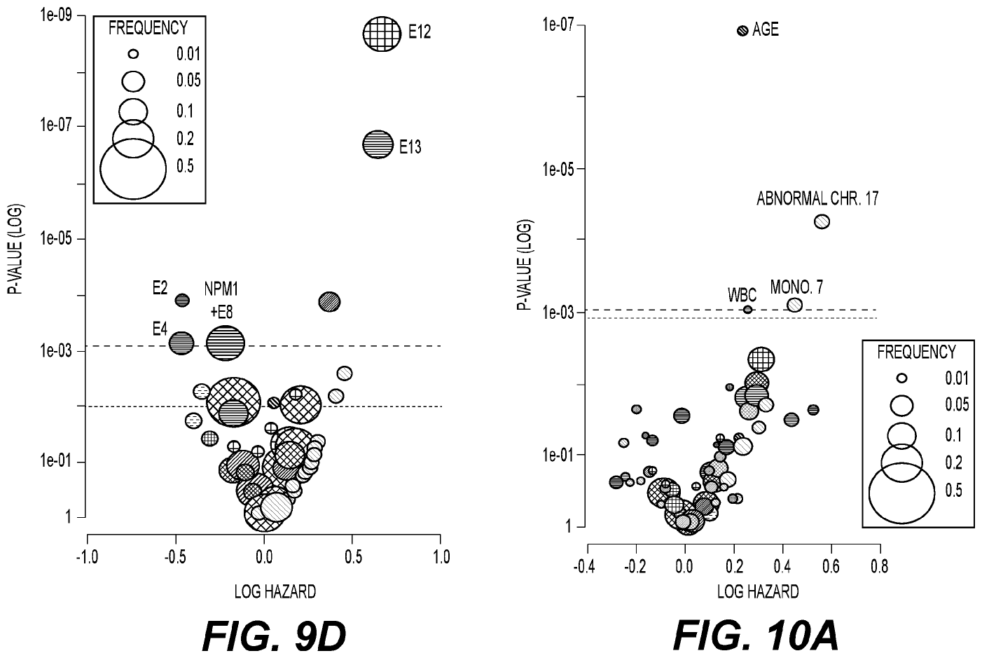

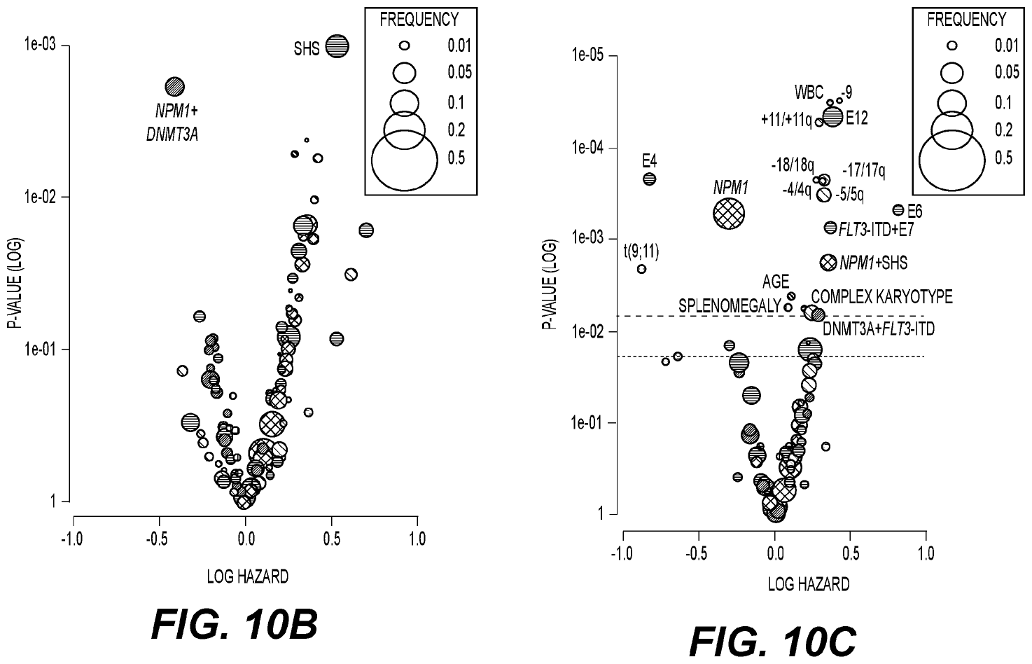

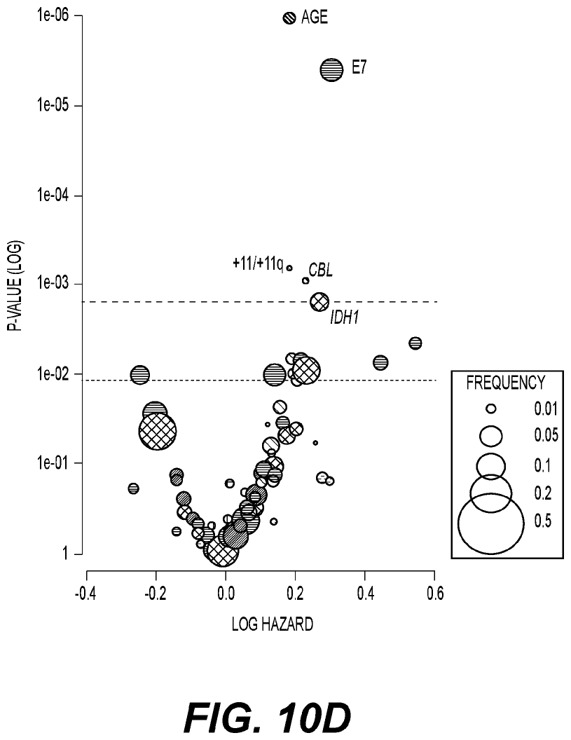

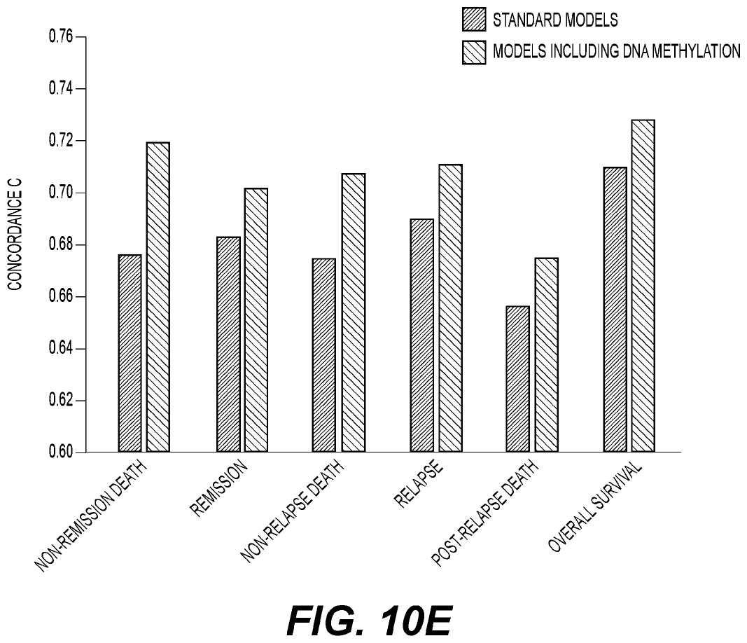

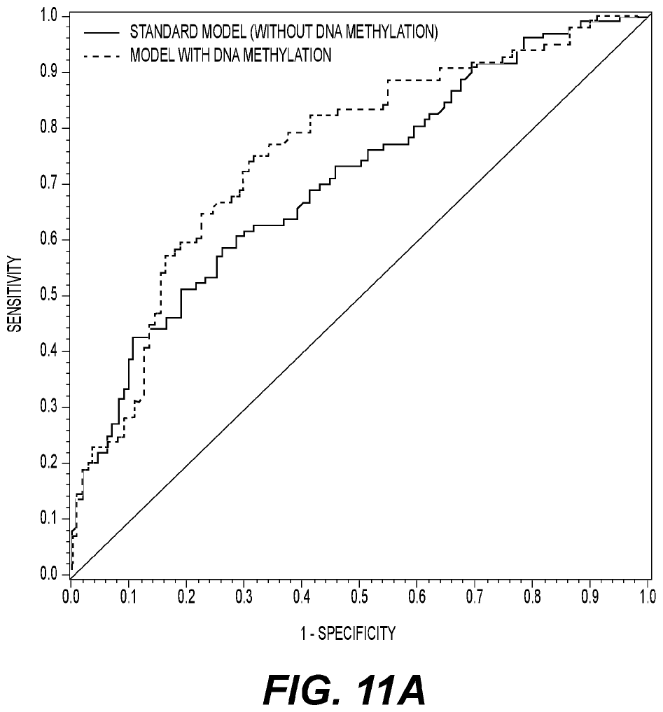

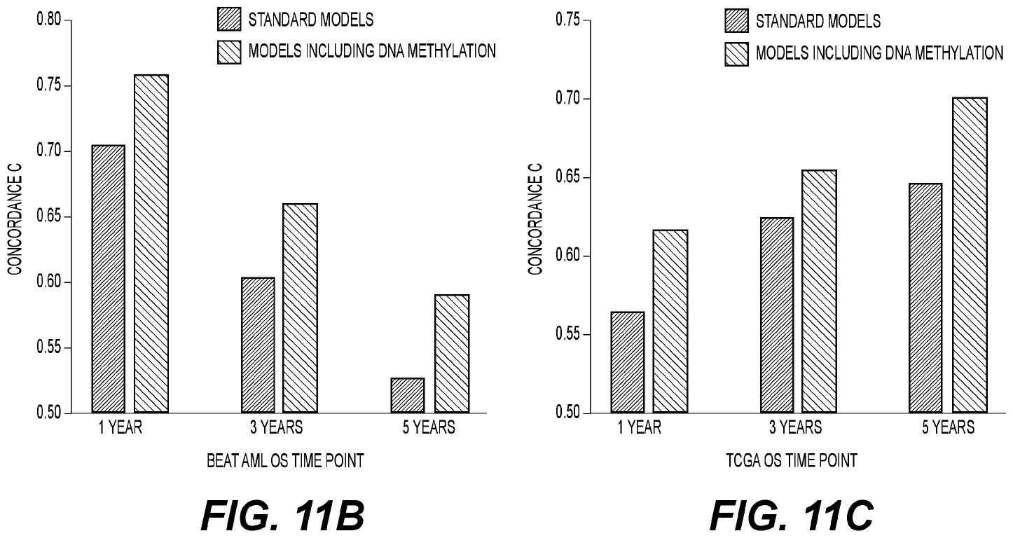

To assess the impact of DNA methylation features among the complex array of other prognostic markers including recurrent genetic events, a multistage random effects (RFX) machine learning model developed by Gerstung et al was employed. This approach is capable of combining a large number of recurrent genetic features along with established clinical and demographic prognostic markers (TABLE 5) and outperforms ELN risk classification in the Alliance AML cohort. By training the algorithm with this wide array of features and outcomes across a large cohort of patients, the algorithm agnostically weights the most important features to build a maximally predictive model for a given clinical endpoint. Here, the multistage RFX algorithm was trained using the Alliance cohort to firstly quantify how much each of 115 individual features across 7 classes contribute to explaining patient-to-patient variation in clinical endpoints, including remission, non-remission death, relapse, non-relapse death, post-relapse death, and OS. When adding epitype and SHS into the algorithm, DNA methylation as a class contributed 30% of the model predicting OS (FIG. 9A). Among all individual features examined, SHS, epitypes and epitype-mutation interactions were among the most significant associations with OS (P<0.0001; FIG. 9B, TABLE 6). DNA methylation notably contributed to all other endpoints examined (FIG. 9C; TABLES 7-11), with various epitypes and/or SHS among the most significant features predicting multiple clinical endpoints (FIGS. 10A, 10B, 10C, and 10D). E12 and E13 were the most significant features for predicting failure to achieve remission; E7, along with age, were the most significant contributors to predicting post-relapse death (FIGS. 9D and 10D). The addition of DNA methylation improved concordance between predicted and actual outcomes for all clinical endpoints examined using internal cross-validation (FIG. 10E). Next the relative predictive power of DNA methylation features was validated in two independent cohorts, the TCGA AML and Beat AML studies, where DNA methylation information and all other requisite data were available. It was found that the inclusion of DNA methylation features increased concordance of predicted versus actual OS in both cohorts (FIG. 11). This work demonstrates that DNA methylation provides important information in predicting patient outcomes, including when combined with a comprehensive set of prognostic markers.

### Integration of DNA Methylation Features to Improve Definition of Favorable Risk AML

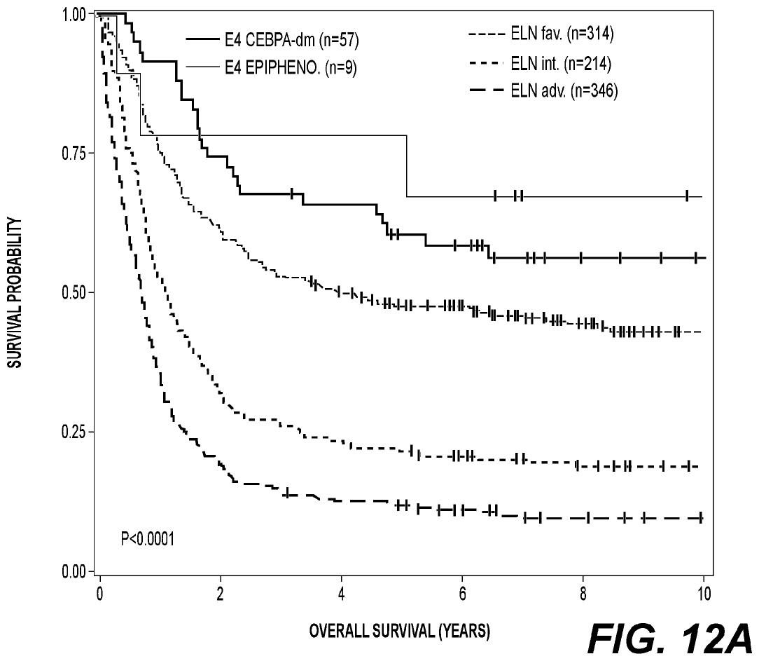

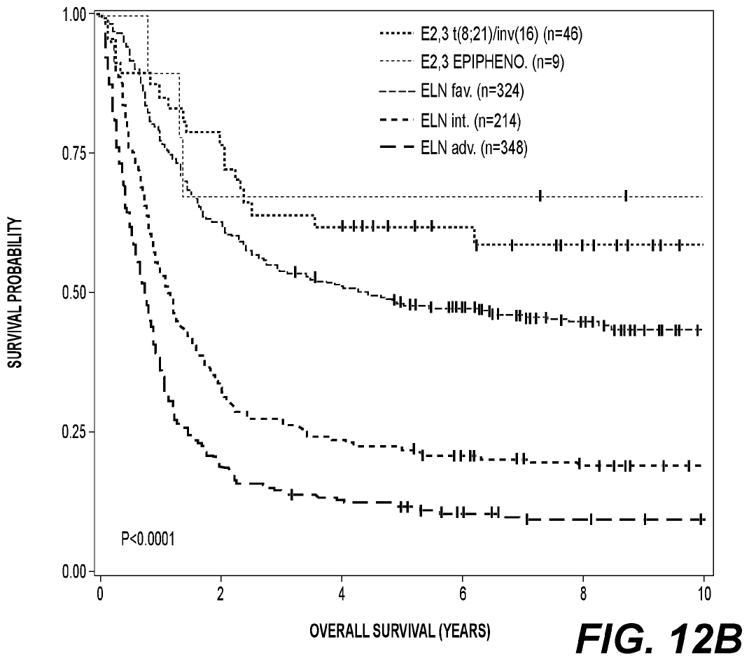

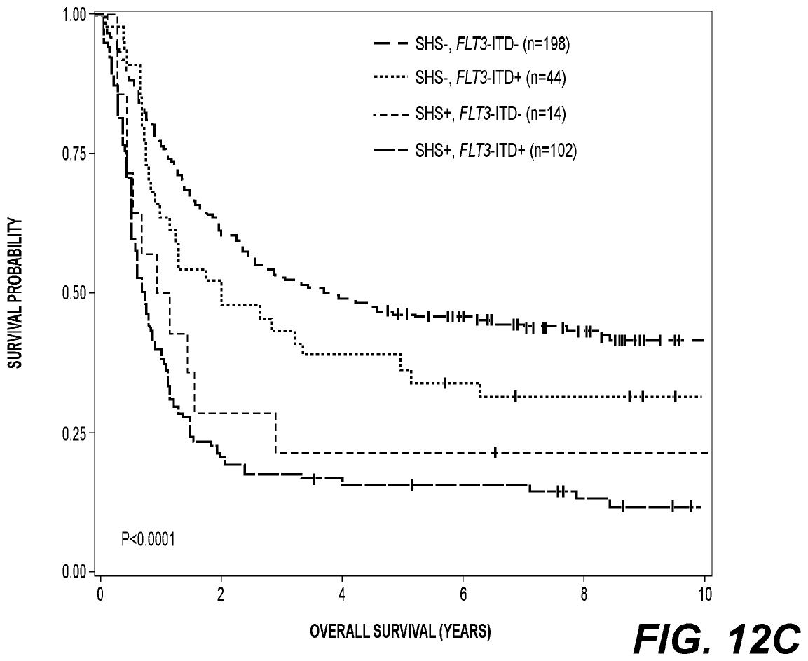

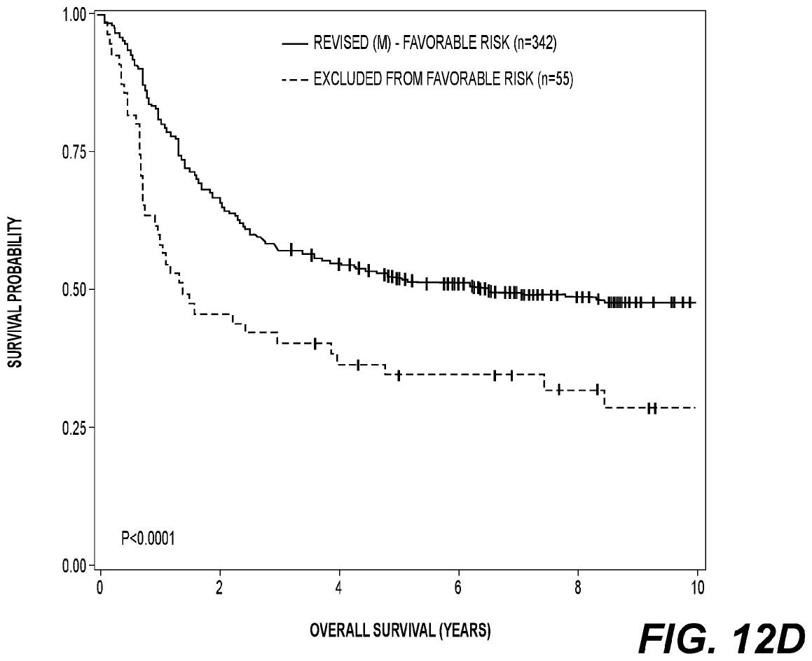

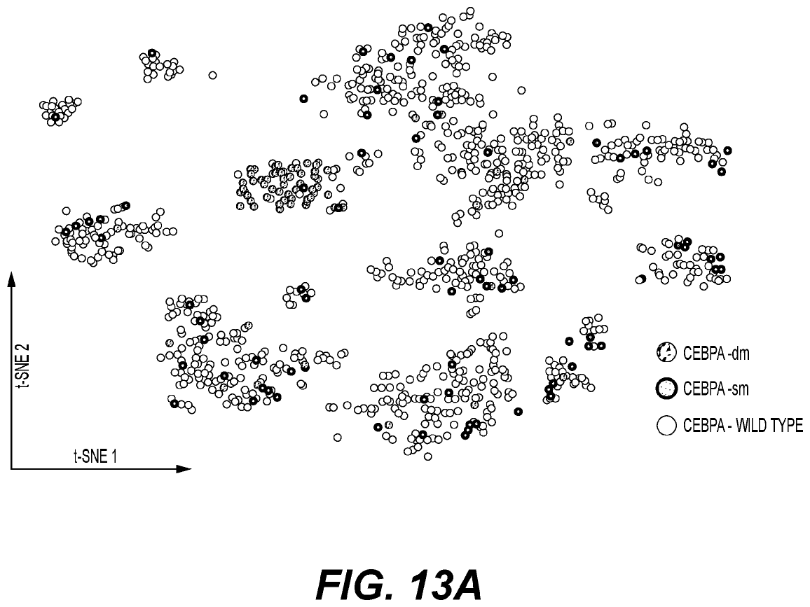

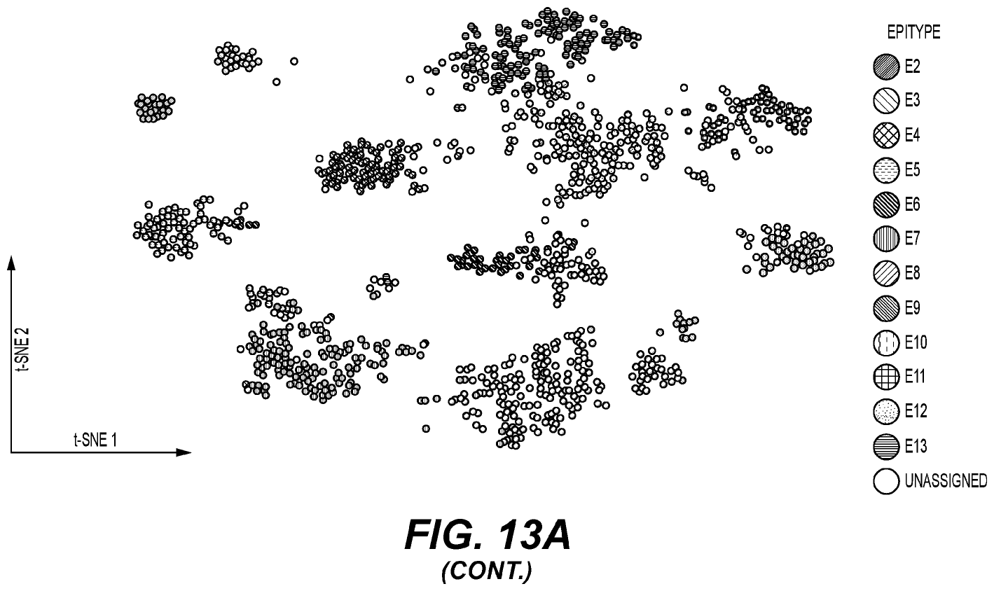

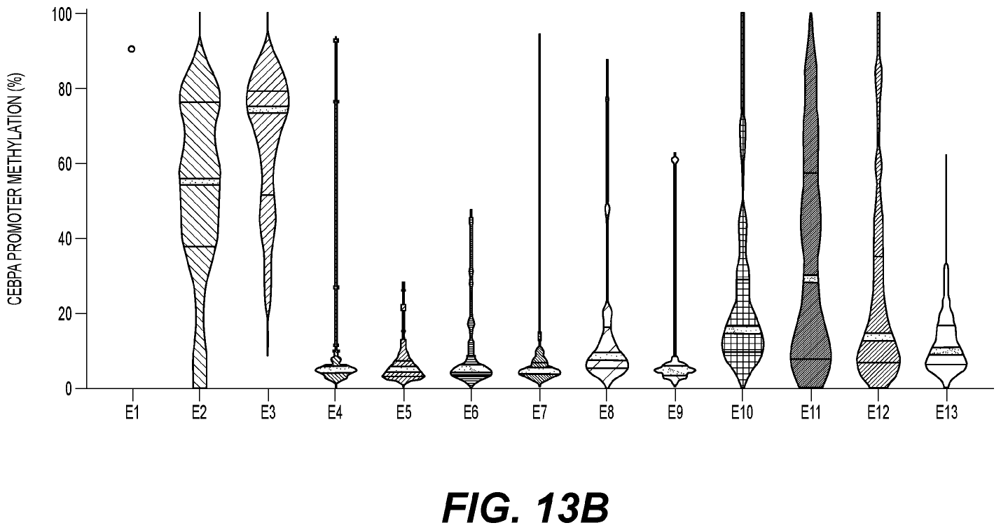

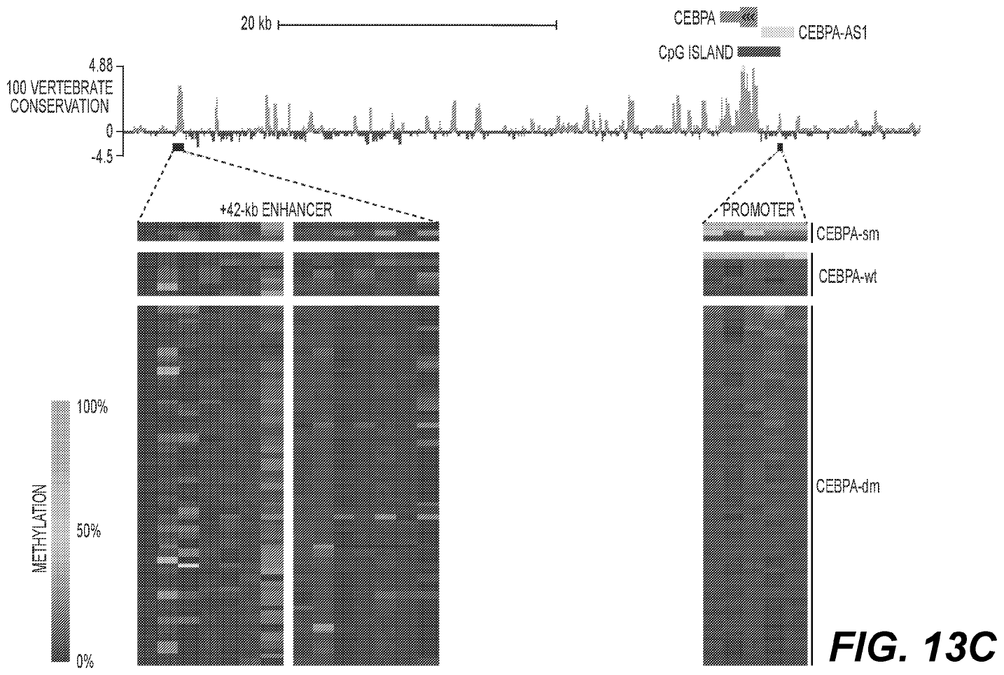

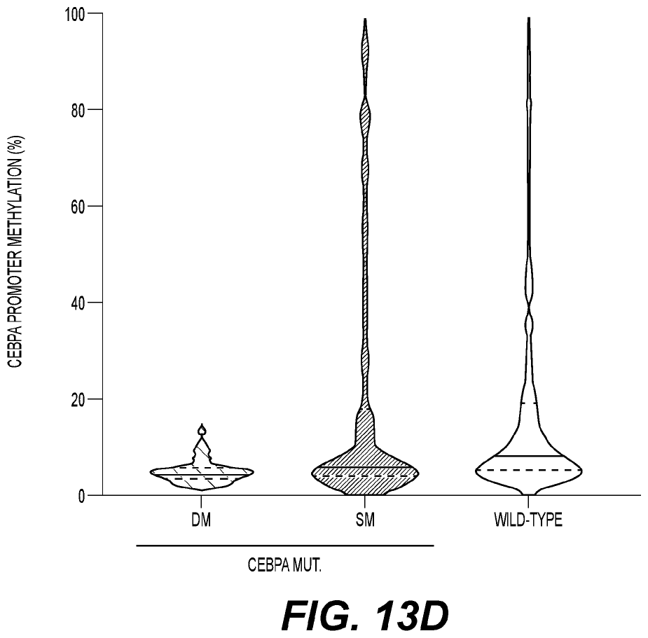

Epiphenocopying of favorable risk markers, such as CEBPA-dm and CBF, could prevent a subset of patients from undergoing unnecessary allo-HCT. We found only 60/72 patients in E4 were CEBPA-dm (FIG. 3C, TABLE 2). Despite all E4 epiphenocopy patients (n=12) being classified as either intermediate (n=6) or adverse risk (n=6), E4 epiphenocopies demonstrated favorable clinical outcomes indistinguishable from CEBPA-dm patients and significantly more favorable than intermediate and adverse risk groups (P<0.0001; FIG. 12A). The underlying basis for E4 epiphenocopies was further investigated and it did not detect an association with monoallelic (single) CEBPA mutations or a role for CEBPA DNA hypermethylation (FIG. 13). For other favorable risk chromosomal rearrangements, 11 patients with methylation patterns were identified with consistent E2-3 but lacking t(8;21) or inv(16) abnormalities. These epiphenocopies also demonstrated favorable outcomes indistinguishable from patients with these chromosomal abnormalities (P<0.0001; FIG. 12B) despite the majority of these patients classified as intermediate risk. It is well appreciated that despite NPM1-mutated patients lacking FLT3-ITD exhibit favorable risk, a subset relapse and die of AML. It was identified that patients displaying SHS-positivity exhibited inferior OS than SHS-negative patients regardless of FLT3-ITD status (P<0.0001; FIG. 12C). Together these results show that epigenetic reprogramming associated with CEBPA, favorable-risk rearrangements, and FLT3-ITD mutations occur despite the lack of these specific genetic events and that risk is more accurately assigned using DNA methylation patterns. Finally, it was sought to integrate the DNA methylation-based classifications to redefine favorable risk in AML. As MLL-SC or SHS+ DNA methylation signatures were associated with inferior outcome (FIGS. 4B and 12C), we identified 55/370 (15%) patients for eviction from the favorable risk category. Next, the epiphenocopies of CBF and CEBPA-dm patients (E2-4) were rescued, which in total resulted in a similar overall number of favorable risk-classified patients (n=342 versus n=370). Kaplan-Meier analysis of survival following restructuring of the favorable risk group showed significantly improved risk prediction using the updated (M-Favorable) risk stratification approach (P<0.0001; FIG. 12D). These analyses illustrate the significant impact of incorporating DNA methylation features into AML risk stratification.

### Discussion

Herein, this disclosure describes a relatively simple approach for generating prognostic information on AML patients that captures many of the standard molecular markers obtained using a variety of individual analyses, including karyotype, cytogenetic and various DNA sequencing approaches. Importantly the DNA methylation-based approach captures information not identified through current diagnostics. Thus, in addition to supporting the identification of a particular genetic marker in a given patient, DNA methylation information supersedes genetic classification in many instances leading to reassignment of patent risk for patients treated with standard intensive chemotherapy. Furthermore, DNA methylation signatures demonstrated broad prognostic importance in comprehensive machine-learning models predicting multiple endpoints, including remission, relapse, and survival.

The finding show that the DNA methylation signature associated with a dominant genetic marker is often more predictive than the marker itself. One possibility for epiphenocopy equivalence is technical type-II (false-negative) error for the genetic marker. Genetic features are well characterized in Alliance cohorts, including central review of karyotype and cytogenetic data for all patients. In addition, duplicate sequencing approaches for major AML-associated genes (detailed in the SM) make an excess type II error in this study contributing to the observed epiphenocopies unlikely. Secondly, epiphenocopies may arise from genomic changes that may be cryptic in standard analyses. Indeed, several publications have identified cryptic structural variants involving the fusion gene partners of inv(16) and t(8;21) were associated with favorable outcome. These genetic alterations can be either too small for standard detection or masked by other complex genetic events. Somatic variants may be missed in standard analyses, especially for loss-of-function mutations, and discerning the functional significance and somatic origin of mutations can be problematic.

A third potential basis for epiphenocopies is functional convergence of lower frequency genetic events or other features that generate DNA methylation signatures congruent with the dominant epitype mutation. It was found that E5 epiphenocopy (MLL-like) patterns were enriched in patients with NPM1, DNMT3A and FLT3-ITD mutations, which together portend poor outcome. E8 (NPM1-like) epiphenocopies, were enriched for ASXL1 mutations, t(6;9) aberrations and 8q gains. As NPM1 mutations are associated with HOX gene activation and all patients in E8 exhibit HOXB hypomethylation, likely the above genomic events are equally triggering aberrant HOX expression. Indeed, some E8 patients clustered alongside E6 (most commonly MLL-rearranged) (FIG. 3A) and MLL rearrangements activate HOX genes. E12 displayed a highly complex mixture of chromosomal aberrations and mutations reminiscent of MDS, and hints at functional convergence of genetic events involved in CK with recurrent mutations in RUNX1 and WT1, as well as inv(3) aberrations. Finally, it was found that SHS epiphenocopies FLT3-ITD, representing alternative mechanisms of STAT pathway activation involving t(9;22), t(8;21) aberrations and WT1 mutations. Conversely, SHS-negativity in FLT3-ITD+ indicates that FLT3-ITD has failed to reprogram of STAT binding sites in these patients. STAT binding site reprogramming may depend on the interaction of FLT3 activation and mutations in other epigenetic modifiers. For example, DNMT3A mutations may destabilize chromatin integrity, facilitating STAT-dependent hypomethylation, whereas hypermethylation resulting from IDH1/2 mutations may avert STAT-dependent reprogramming. Together these results show that epitypes unite various genetic features potentially simplifying genetic complexity.

Combining these results from various analyses, favorable AML has been refined by excluding those with unfavorable DNA methylation signatures and rescuing epiphenocopies of favorable risk genetics, identifying patients that have a significantly more favorable survival (median survival of 6.5 years versus 18 months; FIG. 12D, P<0.0001). This approach for DNA methylation-based classification is rapid and requires low input and is feasible for a clinical setting. Furthermore, AML epitypes are highly stable throughout disease course including following relapse. Incorporation of DNA methylation signatures into risk stratification strategies, including knowledge bank/machine learning approaches, will lead to improved accuracy for predicting benefit associated with HSCT and to support current classification approaches.

### Example 2: Novel Approach for the Identification of HOX Gene-Driven Acute Myeloid Leukemia for Specific Treatment Using Menin Inhibitors

Acute myeloid leukemia (AML) is the most common acute leukemia in adults and has a high mortality rate with standard treatment. An important factor contributing to poor survival is the high degree of underlying biological heterogeneity of AML. Novel precision medicine approaches have been tailored to genetically-defined subsets of AML and have led to improved patient outcomes, including IDH inhibitors for IDH1/2-mutated patents and FLT3 inhibitors targeting FLT3-mutated AML. It has been recognized that rearrangements of the lysine methyltransferase 2A (KMT2A) gene, previously known as mixed-lineage leukemia (MLL), along with mutations in the NPM1 gene, drive activation of HOX genes that critically contribute the pathogenesis in a subset of AML patients. The ability of MLL oncofusions or mutated NPM1 proteins to promote HOX gene activation depends on a cofactor, MENIN, encoding an epigenetic modifier that directly binds to the MLL protein complex and is also critical for leukemogenesis in these patients. This knowledge led to the design of small molecule oral Menin inhibitors and initiation of early phase clinical trials in relapsed AML with positive initial results.

Maximizing the effectiveness and breadth of novel precision therapy approaches for AML relies on accurately predicting the phenotype of tumor cells, which is the product of an array of biological characteristics in addition to genetic mutations. Epigenetic features provide an additional layer of information that dictates how genes are used in a given cell, connecting tumor genetic events to patterns of gene expression and thus controlling the tumor cell phenotype. Using genome-wide profiling of DNA methylation, an epigenetic mark, it was uncovered that genetically-defined subsets of AML can be expanded to include patients that have nearly identical phenotypes despite lacking a specific genetic mutation or rearrangement. These patients were called ‘epiphenocopies’, as the patient retains the DNA methylation pattern associated with the tumor genetic marker yet lacks the specific marker. Indeed, it has been found that epiphenocopies display clinical outcomes in most instances that are indistinguishable from patients that retain the respective genetic mutation. A study of greater than 1,400 AML patients was completed confirming previous studies that have showed NPM/mutations combined with KMT2A rearrangements comprise approximately 35% of AML patients. Importantly, an additional 15-20% of AML patients was identified as epiphenocopy NPM1 mutations and KMT2A rearrangements, substantially expanding the proportion of patients that could targeted with a Menin inhibitor. NPM1 and KMT2A epiphenocopies were also confirmed to display HOX gene activation.

DNA methylation mentioned above is specifically referring to the addition of a methyl group to the 5′ position of cytosine in DNA. In mammals, DNA methylation occurs almost exclusively at cytosine/guanine sequence pairs (CpG dinucleotides). When genes are in an active state, CpGs in the vicinity of a gene promoter are unmethylated, otherwise they are largely methylated throughout the genome. To determine the DNA methylation patterns in AML, 649 patients were assayed and analyzed using Illumina methylation arrays that profile >800,000 CpGs across the human genome. From these data, bioinformatic approaches were used to identify a panel of CpGs that separated patients into 13 distinct methylation subgroups, we termed ‘epitypes’. The MassARRAY iPLEX technology from Agena Biosciences was modified to analyze CpG methylation in a high-throughput, cost-effective manner (termed Methylation-iPLEX). This method was applied to measure the methylation levels of a targeted panel of 42 CpGs in three multiplexed reactions. The accuracy of the AML Methylation-iPLEX was compared and validated to other methods, including genome-wide methylation-based data, and found that it faithfully recapitulated individual CpG methylation levels and epitype classifications. This technique was applied to classify greater than 1,400 AML patients into individual epitypes, resulting in >90% of cases classified.

This invention surrounds the concept that 6/13 epitypes are associated with a high proportion of patients harboring NPM1 mutations or KMT2A rearrangements, however not all patients in these epitypes have these mutations, and thus are designated epiphenocopies as described above. These patients comprise >60% of all AML patients analyzed. They distinctly show loss of DNA methylation at HOX genomic loci and expression of HOX genes regardless of genetic profiles, making DNA methylation-based identification an ideal approach for selection of patients for treatment with a Menin inhibitor.

The clear advantage of this approach is identifying patients suitable for Menin inhibitor treatment regardless of genetic mutations, substantially increasing the proportion of overall patients targetable. Furthermore, measuring DNA methylation epitype is rapid (can be completed in less than a workday), requires very little input material, is cost-effective, robust and high-throughput (if needed). This improves on existing diagnostic approaches measuring genetic and gene expression features. It has been shown that measurement of AML epitype is transferrable to other platforms involving various sequencing technologies. In summary, this invention increases the proportion of AML patients that can benefit from targeted Menin inhibition establishing a useful companion biomarker for the use of Menin inhibitors.

In addition to the 13 DNA methylation subtypes (epitypes), a DNA methylation signature was identified that is associated with FLT3-ITD mutations. FLT3-ITD mutations in AML are associated with poor overall outcomes, including frequent relapse and inferior disease-free and overall survival. Patients with FL73-ITD mutations are treated with specific small molecule inhibitors that target/block FLT3. The analysis of the FLT3-ITD mutation-associated signature show evidence of STAT pathway activation, thus the signature was termed the STAT hypomethylation signature (SHS). The SHS signature is also found to be present in a subset of patients that lack FLT3-ITD mutations, i.e. epiphenocopies of FLT3-ITD. These epiphenocopies display poor outcomes similar to patients with FLT3-ITD mutations. As SHS+ patient in the absence of FLT-ITD show evidence of FLT3/STAT pathway activation, these patients will be enriched in responders to therapies that block FLT3 or other therapies that target the STAT pathway.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
Annotation of CpG panel targeted by the AML Epityping Me-iPLEX assay

| | | | | | Position | Matched |
|---|---|---|---|---|---|---|
| | | | | | relative | to |
| Assay | Gene | Illumina | | Position | to | Illumina |
| Name | Symbol | ID | Chromosome | (hg19) | gene | cg |
| ACOT7 | ACOT7 | cg16034168 | 1 | 6336711 | Body | Y |
| HIVEP3 | HIVEP3 | cg03884592 | 1 | 42384474 | 1stExon | Y |
| AIM2 | AIM2 | cg17515347 | 1 | 159047163 | TSS1500 | Y |
| CD34.1 | CD34 | cg03583857 | 1 | 208085022 | TSS1500 | Y |
| CD34.2 | CD34 | cg26266618 | 1 | 208085043 | TSS1500 | Y |
| CHML | CHML | cg15775914 | 1 | 241799084 | 1stExon | Y |
| DNMT3A.2 | DNMT3A | cg23903708 | 2 | 25527266 | Body | Y |
| DNMT3A.1 | DNMT3A | cg10239163 | 2 | 25527366 | Body | Y |
| ALS2CL | ALS2CL | cg25104512 | 3 | 46735454 | TSS1500 | Y |
| XXYLT1 | XXYLT1 | cg21937377 | 3 | 194868750 | Body | Y |
| TM4SF19 | TM4SF19 | cg01883662 | 3 | 196065289 | TSS200 | Y |
| RGS12 | RGS12 | cg01919885 | 4 | 3365330 | Body | Y |
| LRPAP1 | LRPAP1 | cg04857395 | 4 | 3516637 | Body | Y |
| PPP1R18 | PPP1R18 | cg25659902 | 6 | 30652202 | Body | Y |
| HMGA1 | HMGA1 | cg20294304 | 6 | 34203153 | TSS1500 | N |
| TULP4 | TULP4 | cg00393348 | 6 | 158733508 | TSS200 | Y |
| ZSCAN25 | ZSCAN25 | cg07375256 | 7 | 99222196 | Body | Y |
| GIMAP7 | GIMAP7 | cg08637514 | 7 | 150212707 | 5′UTR | Y |
| PRKAG2 | PRKAG2 | cg17192599 | 7 | 151504864 | 5′UTR | Y |
| CELF2 | CELF2 | cg11002119 | 10 | 11137788 | Body | Y |
| BEND7 | BEND7 | cg 19695507 | 10 | 13526193 | Body | Y |
| MLLT10 | MLLT10 | cg12225526 | 10 | 21796388 | 5′Upstream | N |
| ZNF438 | ZNF438 | cg00428179 | 10 | 31322131 | TSS1500 | Y |
| HCCA2 | HCCA2 | cg20299572 | 11 | 1750763 | Body | Y |
| WT1 | WT1 | cg03052301 | 11 | 32459954 | Body | Y |
| CTTN | CTTN | cg09352338 | 11 | 70266139 | Body | Y |
| MED13L | MED13L | cg12220034 | 12 | 116457644 | Body | Y |
| REC8 | REC8 | cg18628371 | 14 | 24641189 | TSS200 | N |
| CCDC9B | CCDC9B | cg12732548 | 15 | 40631573 | Body | Y |
| ESRP2 | ESRP2 | cg08694699 | 16 | 68270282 | TSS200 | Y |
| ZZEF1 | ZZEF1 | cg08166720 | 17 | 3960489 | Body | Y |
| HOXB3.1 | HOXB3 | cg01990102 | 17 | 46646444 | Body | Y |
| HOXB3.2.1 | HOXB3 | cg13293524 | 17 | 46651822 | TSS200 | Y |
| HOXB3.2.2 | HOXB3 | cg04117801 | 17 | 46651867 | TSS200 | Y |
| HOXB3.3 | HOXB3 | cg24767968 | 17 | 46651945 | TSS200 | Y |
| HOXB-AS3.1 | HOXB-AS3 | cg07676709 | 17 | 46673442 | TSS200 | Y |
| HOXB-AS3.2 | HOXB-AS3 | cg21816532 | 17 | 46680006 | Body | N |
| PALM.1 | PALM | cg27183173 | 19 | 728040 | Body | Y |
| PALM.2 | PALM | cg07876162 | 19 | 728176 | Body | Y |
| MEF2B | MEF2B | cg 12558012 | 19 | 19281197 | Body | N |
| PDYN-AS1 | PDYN-AS1 | cg07210840 | 20 | 1927816 | 5′UTR | Y |
| A4GALT | A4GALT | cg15429214 | 22 | 43166281 | 5′Upstream | N |
| MIRLET7BHG | MIRLET7BHG | cg18066206 | 22 | 46459890 | Body | N |

TABLE 2
Distribution of genetic aberrations between epigenetic subtypes

| Mutation | | Total | E1 | E2 | E3 | E4 | E5 | E6 |
|---|---|---|---|---|---|---|---|---|
| Total | Status | n = 1,105 | n = 1 | n = 29 | n = 26 | n = 80 | n = 95 | n = 46 |
| ASXL1 | Mut. (%) | 74 (7) | 0 (0) | 0 (0) | 0 (0) | 2 (3) | 5 (5) | 1 (2) |
| | WT (%) | 1031 (93) | 1 (100) | 29 (100) | 26 (100) | 78 (98) | 90 (95) | 45 (98) |
| BCOR | Mut. (%) | 67 (6) | 0 (0) | 0 (0) | 0 (0) | 1 (1) | 2 (2) | 4 (9) |
| | WT (%) | 1038 (94) | 1 (100) | 29 (100) | 26 (100) | 79 (99) | 93 (98) | 42 (91) |
| BRAF | Mut. (%) | 7 (1) | 0 (0) | 0 (0) | 0 (0) | 0 (0) | 0 (0) | 0 (0) |
| | WT (%) | 1098 (99) | 1 (100) | 29 (100) | 26 (100) | 80 (100) | 95 (100) | 46 (100) |
| CBL | Mut. (%) | 23 (2) | 0 (0) | 0 (0) | 0 (0) | 1 (1) | 1 (1) | 0 (0) |
| | WT (%) | 1082 (98) | 1 (100) | 29 (100) | 26 (100) | 79 (99) | 94 (99) | 46 (100) |
| DNMT3A | Mut. (%) | 280 (25) | 0 (0) | 0 (0) | 0 (0) | 4 (5) | 21 (22) | 0 (0) |
| | WT (%) | 825 (75) | 1 (100) | 29 (100) | 26 (100) | 76 (95) | 74 (78) | 46 (100) |
| ETV6 | Mut. (%) | 26 (2) | 0 (0) | 0 (0) | 0 (0) | 0 (0) | 1 (1) | 2 (4) |
| | WT (%) | 1079 (98) | 1 (100) | 29 (100) | 26 (100) | 80 (100) | 94 (99) | 44 (96) |
| EZH2 | Mut. (%) | 28 (3) | 0 (0) | 0 (0) | 0 (0) | 5 (6) | 1 (1) | 1 (2) |
| | WT (%) | 1077 (97) | 1 (100) | 29 (100) | 26 (100) | 75 (94) | 94 (99) | 45 (98) |
| FBXW7 | Mut. (%) | 1 (0) | 0 (0) | 0 (0) | 0 (0) | 0 (0) | 0 (0) | 0 (0) |
| | WT (%) | 1099 (100) | 1 (100) | 29 (100) | 26 (100) | 80 (100) | 95 (100) | 46 (100) |
| GATA2 | Mut. (%) | 61 (6) | 0 (0) | 0 (0) | 0 (0) | 30 (38) | 2 (2) | 4 (9) |
| | WT (%) | 1044 (94) | 1 (100) | 29 (100) | 26 (100) | 50 (63) | 93 (98) | 42 (91) |
| IDH1 | Mut. (%) | 97 (9) | 0 (0) | 0 (0) | 0 (0) | 2 (3) | 3 (3) | 1 (2) |
| | WT (%) | 1007 (91) | 1 (100) | 29 (100) | 26 (100) | 78 (98) | 92 (97) | 45 (98) |
| IKZF1 | Mut. (%) | 23 (2) | 0 (0) | 1 (3) | 0 (0) | 5 (6) | 1 (1) | 0 (0) |
| | WT (%) | 1082 (98) | 1 (100) | 28 (97) | 26 (100) | 75 (94) | 94 (99) | 46 (100) |
| JAK2 | Mut. (%) | 5 (0) | 0 (0) | 1 (4) | 0 (0) | 0 (0) | 0 (0) | 0 (0) |
| | WT (%) | 1056 (100) | 1 (100) | 27 (96) | 26 (100) | 76 (100) | 92 (100) | 43 (100) |
| KIT | Mut. (%) | 42 (4) | 0 (0) | 9 (31) | 4 (15) | 5 (6) | 3 (3) | 2 (4) |
| | WT (%) | 1040 (96) | 1 (100) | 20 (69) | 22 (85) | 74 (94) | 90 (97) | 44 (96) |
| KRAS | Mut. (%) | 60 (5) | 0 (0) | 4 (14) | 3 (12) | 4 (5) | 9 (9) | 13 (28) |
| | WT (%) | 1043 (95) | 1 (100) | 25 (86) | 23 (88) | 75 (95) | 86 (91) | 33 (72) |
| MLL | Mut. (%) | 17 (2) | 0 (0) | 1 (3) | 0 (0) | 2 (3) | 1 (1) | 1 (2) |
| | WT (%) | 1088 (98) | 1 (100) | 28 (97) | 26 (100) | 78 (98) | 94 (99) | 45 (98) |
| MPL | Mut. (%) | 12 (1) | 1 (100) | 1 (6) | 1 (5) | 1 (1) | 1 (1) | 0 (0) |
| | WT (%) | 907 (99) | 0 (0) | 15 (94) | 18 (95) | 67 (99) | 75 (99) | 36 (100) |
| NF1 | Mut. (%) | 50 (6) | 0 (0) | 0 (0) | 0 (0) | 2 (3) | 3 (5) | 0 (0) |
| | WT (%) | 725 (94) | 1 (100) | 13 (100) | 18 (100) | 62 (97) | 58 (95) | 26 (100) |
| NPM1 | Mut. (%) | 400 (37) | 0 (0) | 0 (0) | 0 (0) | 1 (1) | 17 (18) | 1 (2) |
| | WT (%) | 692 (63) | 1 (100) | 29 (100) | 26 (100) | 79 (99) | 78 (82) | 45 (98) |
| NRAS | Mut. (%) | 173 (16) | 0 (0) | 2 (7) | 11 (42) | 12 (15) | 12 (13) | 12 (26) |
| | WT (%) | 932 (84) | 1 (100) | 27 (93) | 15 (58) | 68 (85) | 83 (87) | 34 (74) |
| PHF6 | Mut. (%) | 30 (3) | 0 (0) | 1 (3) | 0 (0) | 0 (0) | 0 (0) | 0 (0) |
| | WT (%) | 1075 (97) | 1 (100) | 28 (97) | 26 (100) | 80 (100) | 95 (100) | 46 (100) |
| PTEN | Mut. (%) | 6 (1) | 0 (0) | 0 (0) | 0 (0) | 0 (0) | 1 (1) | 0 (0) |
| | WT (%) | 1099 (99) | 1 (100) | 29 (100) | 26 (100) | 80 (100) | 94 (99) | 46 (100) |
| PTPN11 | Mut. (%) | 104 (9) | 0 (0) | 0 (0) | 2 (8) | 0 (0) | 13 (14) | 2 (4) |
| | WT (%) | 1001 (91) | 1 (100) | 29 (100) | 24 (92) | 80 (100) | 82 (86) | 44 (96) |
| RAD21 | Mut. (%) | 26 (2) | 0 (0) | 1 (3) | 0 (0) | 3 (4) | 1 (1) | 0 (0) |
| | WT (%) | 1079 (98) | 1 (100) | 28 (97) | 26 (100) | 77 (96) | 94 (99) | 46 (100) |
| RUNX1 | Mut. (%) | 116 (10) | 0 (0) | 0 (0) | 0 (0) | 2 (3) | 3 (3) | 4 (9) |
| | WT (%) | 989 (90) | 1 (100) | 29 (100) | 26 (100) | 78 (98) | 92 (97) | 42 (91) |
| SF1 | Mut. (%) | 9 (1) | 0 (0) | 0 (0) | 0 (0) | 0 (0) | 0 (0) | 0 (0) |
| | WT (%) | 1096 (99) | 1 (100) | 29 (100) | 26 (100) | 80 (100) | 95 (100) | 46 (100) |
| SF3A1 | Mut. (%) | 10 (1) | 0 (0) | 0 (0) | 1 (4) | 1 (1) | 0 (0) | 0 (0) |
| | WT (%) | 1095 (99) | 1 (100) | 29 (100) | 25 (96) | 79 (99) | 95 (100) | 46 (100) |
| SF3B1 | Mut. (%) | 40 (4) | 0 (0) | 1 (3) | 1 (4) | 1 (1) | 1 (1) | 1 (2) |
| | WT (%) | 1065 (96) | 1 (100) | 28 (97) | 25 (96) | 79 (99) | 94 (99) | 45 (98) |
| SFRS2 | Mut. (%) | 78 (7) | 0 (0) | 0 (0) | 0 (0) | 4 (5) | 1 (1) | 1 (2) |
| | WT (%) | 1021 (93) | 1 (100) | 29 (100) | 25 (100) | 76 (95) | 94 (99) | 45 (98) |
| STAG2 | Mut. (%) | 35 (3) | 0 (0) | 0 (0) | 0 (0) | 1 (1) | 0 (0) | 2 (4) |
| | WT (%) | 1070 (97) | 1 (100) | 29 (100) | 26 (100) | 79 (99) | 95 (100) | 44 (96) |
| TET2 | Mut. (%) | 145 (13) | 0 (0) | 1 (3) | 0 (0) | 6 (8) | 8 (8) | 5 (11) |
| | WT (%) | 960 (87) | 1 (100) | 28 (97) | 26 (100) | 74 (93) | 87 (92) | 41 (89) |
| TP53 | Mut. (%) | 94 (9) | 0 (0) | 0 (0) | 0 (0) | 0 (0) | 12 (13) | 1 (2) |
| | WT (%) | 1011 (91) | 1 (100) | 29 (100) | 26 (100) | 80 (100) | 83 (87) | 45 (98) |
| U2AF1 | Mut. (%) | 43 (4) | 0 (0) | 0 (0) | 0 (0) | 2 (3) | 5 (5) | 3 (7) |
| | WT (%) | 1062 (96) | 1 (100) | 29 (100) | 26 (100) | 78 (98) | 90 (95) | 43 (93) |
| WT1 | Mut. (%) | 94 (9) | 0 (0) | 0 (0) | 3 (12) | 13 (16) | 0 (0) | 0 (0) |
| | WT (%) | 1011 (91) | 1 (100) | 29 (100) | 23 (88) | 67 (84) | 95 (100) | 46 (100) |
| ZRSR2 | Mut. (%) | 53 (5) | 0 (0) | 1 (3) | 0 (0) | 1 (1) | 4 (4) | 3 (7) |
| | WT (%) | 1052 (95) | 1 (100) | 28 (97) | 26 (100) | 79 (99) | 91 (96) | 43 (93) |
| CEBPA-sm | Mut. (%) | 62 (7) | 0 (0) | 1 (14) | 0 (0) | 3 (4) | 5 (6) | 2 (5) |
| | WT (%) | 831 (93) | 1 (100) | 6 (86) | 2 (100) | 68 (96) | 72 (94) | 37 (95) |
| CEBPA-dm | Mut. (%) | 76 (9) | 0 (0) | 1 (14) | 0 (0) | 60 (85) | 1 (1) | 1 (3) |
| | WT (%) | 817 (91) | 1 (100) | 6 (86) | 2 (100) | 11 (15) | 76 (99) | 38 (97) |
| FLT3-ITD | Mut. (%) | 239 (22) | 1 (100) | 0 (0) | 0 (0) | 10 (13) | 15 (16) | 2 (4) |
| | WT (%) | 865 (78) | 0 (0) | 29 (100) | 26 (100) | 70 (88) | 80 (84) | 43 (96) |
| FLT3-TKD | Mut. (%) | 105 (10) | 0 (0) | 1 (4) | 2 (8) | 1 (1) | 15 (16) | 0 (0) |
| | WT (%) | 984 (90) | 1 (100) | 27 (96) | 24 (92) | 77 (99) | 80 (84) | 45 (100) |
| IDH2 p172 | Mut. (%) | 38 (3) | 0 (0) | 0 (0) | 0 (0) | 0 (0) | 0 (0) | 0 (0) |
| | WT (%) | 1067 (97) | 1 (100) | 29 (100) | 26 (100) | 80 (100) | 95 (100) | 46 (100) |
| IDH2 p140 | Mut. (%) | 97 (9) | 0 (0) | 0 (0) | 0 (0) | 0 (0) | 4 (4) | 1 (2) |
| | WT (%) | 1008 (91) | 1 (100) | 29 (100) | 26 (100) | 80 (100) | 91 (96) | 45 (98) |
| inv(3)/t(3; 3) | Pos. | 22 (2) | 0 (0) | 0 (0) | 0 (0) | 0 (0) | 0 (0) | 0 (0) |
| | Neg. | 1083 (98) | 1 (100) | 29 (100) | 26 (100) | 80 (100) | 95 (100) | 46 (100) |
| t(9; 22) | Pos. | 14 (1) | 0 (0) | 0 (0) | 0 (0) | 1 (1) | 0 (0) | 0 (0) |
| | Neg. | 1091 (99) | 1 (100) | 29 (100) | 26 (100) | 79 (99) | 95 (100) | 46 (100) |
| Monosomy | Pos. | 90 (8) | 0 (0) | 0 (0) | 0 (0) | 2 (3) | 5 (5) | 0 (0) |
| 5, del(5q) | Neg. | 1015 (92) | 1 (100) | 29 (100) | 26 (100) | 78 (98) | 90 (95) | 46 (100) |
| monosomy | Pos. | 89 (8) | 0 (0) | 0 (0) | 0 (0) | 0 (0) | 3 (3) | 0 (0) |
| 7 | Neg. | 1016 (92) | 1 (100) | 29 (100) | 26 (100) | 80 (100) | 92 (97) | 46 (100) |
| del(7q) | Pos. | 55 (5) | 0 (0) | 2 (7) | 0 (0) | 2 (3) | 1 (1) | 2 (4) |
| | Neg. | 1050 (95) | 1 (100) | 27 (93) | 26 (100) | 78 (98) | 94 (99) | 44 (96) |
| Abnormal | Pos. | 12 (1) | 0 (0) | 0 (0) | 0 (0) | 2 (3) | 4 (4) | 1 (2) |
| chr. 7 | Neg. | 1093 (99) | 1 (100) | 29 (100) | 26 (100) | 78 (98) | 91 (96) | 45 (98) |
| (other) | | | | | | | | |
| Plus 8, +8q | Pos. | 132 (12) | 1 (100) | 2 (7) | 2 (8) | 0 (0) | 33 (35) | 4 (9) |
| | Neg. | 973 (88) | 0 (0) | 27 (93) | 24 (92) | 80 (100) | 62 (65) | 42 (91) |
| del(9q) | Pos. | 23 (2) | 0 (0) | 1 (3) | 0 (0) | 5 (6) | 3 (3) | 1 (2) |
| | Neg. | 1082 (98) | 1 (100) | 28 (97) | 26 (100) | 75 (94) | 92 (97) | 45 (98) |
| Abnormal | Pos. | 42 (4) | 0 (0) | 0 (0) | 0 (0) | 1 (1) | 7 (7) | 0 (0) |
| chr. 12 | Neg. | 1063 (96) | 1 (100) | 29 (100) | 26 (100) | 79 (99) | 88 (93) | 46 (100) |
| Plus 13 | Pos. | 24 (2) | 0 (0) | 0 (0) | 0 (0) | 0 (0) | 2 (2) | 0 (0) |
| | Neg. | 1081 (98) | 1 (100) | 29 (100) | 26 (100) | 80 (100) | 93 (98) | 46 (100) |
| Monosomy | Pos. | 69 (6) | 0 (0) | 0 (0) | 0 (0) | 0 (0) | 2 (2) | 0 (0) |
| 17, | Neg. | 1036 (94) | 1 (100) | 29 (100) | 26 (100) | 80 (100) | 93 (98) | 46 (100) |
| abnormal | | | | | | | | |
| chr. 17p | | | | | | | | |
| Monosomy | Pos. | 27 (2) | 0 (0) | 0 (0) | 0 (0) | 0 (0) | 0 (0) | 0 (0) |
| 18, del(18q) | Neg. | 1078 (98) | 1 (100) | 29 (100) | 26 (100) | 80 (100) | 95 (100) | 46 (100) |
| Monosomy | Pos. | 30 (3) | 0 (0) | 0 (0) | 1 (4) | 0 (0) | 2 (2) | 0 (0) |
| 20, del(20q) | Neg. | 1075 (97) | 1 (100) | 29 (100) | 25 (96) | 80 (100) | 93 (98) | 46 (100) |
| Plus 21 | Pos. | 26 (2) | 0 (0) | 2 (7) | 0 (0) | 0 (0) | 2 (2) | 2 (4) |
| | Neg. | 1079 (98) | 1 (100) | 27 (93) | 26 (100) | 80 (100) | 93 (98) | 44 (96) |
| Plus 22 | Pos. | 26 (2) | 0 (0) | 0 (0) | 5 (19) | 0 (0) | 1 (1) | 1 (2) |
| | Neg. | 1079 (98) | 1 (100) | 29 (100) | 21 (81) | 80 (100) | 94 (99) | 45 (98) |
| Minus Y | Pos. | 35 (3) | 0 (0) | 13 (45) | 1 (4) | 2 (3) | 2 (2) | 2 (4) |
| | Neg. | 1070 (97) | 1 (100) | 16 (55) | 25 (96) | 78 (98) | 93 (98) | 44 (96) |
| t(8; 21) | Pos. | 22 (2) | 0 (0) | 21 (72) | 1 (4) | 0 (0) | 0 (0) | 0 (0) |
| | Neg. | 1083 (98) | 1 (100) | 8 (28) | 25 (96) | 80 (100) | 95 (100) | 46 (100) |
| inv(16) | Pos. | 24 (2) | 0 (0) | 1 (3) | 23 (88) | 0 (0) | 0 (0) | 0 (0) |
| | Neg. | 1081 (98) | 1 (100) | 28 (97) | 3 (12) | 80 (100) | 95 (100) | 46 (100) |
| t(6; 9) | Pos. | 7 (1) | 0 (0) | 0 (0) | 0 (0) | 0 (0) | 1 (1) | 0 (0) |
| | Neg. | 1098 (99) | 1 (100) | 29 (100) | 26 (100) | 80 (100) | 94 (99) | 46 (100) |
| Plus 11, +11q | Pos. | 29 (3) | 0 (0) | 0 (0) | 0 (0) | 0 (0) | 1 (1) | 0 (0) |
| | Neg. | 1076 (97) | 1 (100) | 29 (100) | 26 (100) | 80 (100) | 94 (99) | 46 (100) |
| Abnormal | Pos. | 17 (2) | 0 (0) | 0 (0) | 0 (0) | 0 (0) | 2 (2) | 0 (0) |
| chr. 4 | Neg. | 1088 (98) | 1 (100) | 29 (100) | 26 (100) | 80 (100) | 93 (98) | 46 (100) |
| Complex | Pos. | 131 (12) | 0 (0) | 0 (0) | 1 (4) | 0 (0) | 14 (15) | 2 (4) |
| karyotype | Neg. | 974 (88) | 1 (100) | 29 (100) | 25 (96) | 80 (100) | 81 (85) | 44 (96) |
| t(9; 11) | Pos. | 34 (3) | 0 (0) | 0 (0) | 0 (0) | 1 (1) | 24 (25) | 7 (15) |
| | Neg. | 1071 (97) | 1 (100) | 29 (100) | 26 (100) | 79 (99) | 71 (75) | 39 (85) |
| t(v; 11) | Pos. | 49 (4) | 0 (0) | 0 (0) | 0 (0) | 2 (3) | 14 (15) | 24 (52) |
| (other) | Neg. | 1056 (96) | 1 (100) | 29 (100) | 26 (100) | 78 (98) | 81 (85) | 22 (48) |

TABLE 2
Distribution of genetic aberrations between epigenetic subtypes

| Mutation | | E7 | E8 | ES | E10 | E11 | E12 | E13 | |
|---|---|---|---|---|---|---|---|---|---|
| Total | Status | n = 153 | n = 237 | n = 22 | n = 42 | n = 63 | n = 193 | n = 118 | P-value |
| ASXL1 | Mut. (%) | 1 | 4 | 0 | 2 | 13 | 24 | 22 | <0.0001 |
| | | (1) | (2) | (0) | (5) | (21) | (12) | (19) | |
| | WT (%) | 152 | 233 | 22 | 40 | 50 | 169 | 96 | |
| | | (99) | (98) | (100) | (95) | (79) | (88) | (81) | |
| BCOR | Mut. (%) | 7 | 7 | 0 | 0 | 10 | 25 | 11 | <0.0001 |
| | | (5) | (3) | (0) | (0) | (16) | (13) | (9) | |
| | WT (%) | 146 | 230 | 22 | 42 | 53 | 168 | 107 | |
| | | (95) | (97) | (100) | (100) | (84) | (87) | (91) | |
| BRAF | Mut. (%) | 4 | 0 | 1 | 0 | 0 | 1 | 1 | 0.0895 |
| | | (3) | (0) | (5) | (0) | (0) | (1) | (1) | |
| | WT (%) | 149 | 237 | 21 | 42 | 63 | 192 | 117 | |
| | | (97) | (100) | (95) | (100) | (100) | (99) | (99) | |
| CBL | Mut. (%) | 5 | 7 | 0 | 0 | 0 | 6 | 3 | 0.7286 |
| | | (3) | (3) | (0) | (0) | (0) | (3) | (3) | |
| | WT (%) | 148 | 230 | 22 | 42 | 63 | 187 | 115 | |
| | | (97) | (97) | (100) | (100) | (100) | (97) | (97) | |
| DNMT3A | Mut. (%) | 126 | 45 | 4 | 6 | 16 | 18 | 40 | <0.0001 |
| | | (82) | (19) | (18) | (14) | (25) | (9) | (34) | |
| | WT (%) | 27 | 192 | 18 | 36 | 47 | 175 | 78 | |
| | | (18) | (81) | (82) | (86) | (75) | (91) | (66) | |
| ETV6 | Mut. (%) | 4 | 3 | 0 | 1 | 1 | 12 | 2 | 0.0772 |
| | | (3) | (1) | (0) | (2) | (2) | (6) | (2) | |
| | WT (%) | 149 | 234 | 22 | 41 | 62 | 181 | 116 | |
| | | (97) | (99) | (100) | (98) | (98) | (94) | (98) | |
| EZH2 | Mut. (%) | 3 | 3 | 2 | 0 | 2 | 5 | 6 | 0.162 |
| | | (2) | (1) | (9) | (0) | (3) | (3) | (5) | |
| | WT (%) | 150 | 234 | 20 | 42 | 61 | 188 | 112 | |
| | | (98) | (99) | (91) | (100) | (97) | (97) | (95) | |
| FBXW7 | Mut. (%) | 0 | 0 | 0 | 0 | 0 | 1 | 0 | 0.9663 |
| | | (0) | (0) | (0) | (0) | (0) | (1) | (0) | |
| | WT (%) | 152 | 236 | 22 | 42 | 61 | 191 | 118 | |
| | | (100) | (100) | (100) | (100) | (100) | (99) | (100) | |
| GATA2 | Mut. (%) | 4 | 8 | 0 | 0 | 1 | 9 | 3 | <0.0001 |
| | | (3) | (3) | (0) | (0) | (2) | (5) | (3) | |
| | WT (%) | 149 | 229 | 22 | 42 | 62 | 184 | 115 | |
| | | (97) | (97) | (100) | (100) | (98) | (95) | (97) | |
| IDH1 | Mut. (%) | 7 | 24 | 1 | 21 | 16 | 8 | 14 | <0.0001 |
| | | (5) | (10) | (5) | (50) | (26) | (4) | (12) | |
| | WT (%) | 146 | 213 | 21 | 21 | 46 | 185 | 104 | |
| | | (95) | (90) | (95) | (50) | (74) | (96) | (88) | |
| IKZF1 | Mut. (%) | 1 | 1 | 0 | 0 | 3 | 10 | 1 | 0.0067 |
| | | (1) | (0) | (0) | (0) | (5) | (5) | (1) | |
| | WT (%) | 152 | 236 | 22 | 42 | 60 | 183 | 117 | |
| | | (99) | (100) | (100) | (100) | (95) | (95) | (99) | |
| JAK2 | Mut. (%) | 0 | 1 | 0 | 1 | 1 | 1 | 0 | 0.3299 |
| | | (0) | (0) | (0) | (2) | (2) | (1) | (0) | |
| | WT (%) | 150 | 228 | 21 | 40 | 57 | 183 | 112 | |
| | | (100) | (100) | (100) | (98) | (98) | (99) | (100) | |
| KIT | Mut. (%) | 5 | 7 | 0 | 0 | 0 | 6 | 1 | <0.0001 |
| | | (3) | (3) | (0) | (0) | (0) | (3) | (1) | |
| | WT (%) | 144 | 223 | 22 | 41 | 60 | 185 | 114 | |
| | | (97) | (97) | (100) | (100) | (100) | (97 | (99) | |
| KRAS | Mut. (%) | 7 | 8 | 2 | 0 | 2 | 5 | 3 | <0.0001 |
| | | (5) | (3) | (9) | (0) | (3) | (3) | (3) | |
| | WT (%) | 146 | 229 | 20 | 42 | 60 | 188 | 115 | |
| | | (95) | (97) | (91) | (100) | (97) | (97) | (97) | |
| MLL | Mut. (%) | 2 | 2 | 0 | 1 | 3 | 4 | 0 | 0.6373 |
| | | (1) | (1) | (0) | (2) | (5) | (2) | (0) | |
| | WT (%) | 151 | 235 | 22 | 41 | 60 | 189 | 118 | |
| | | (99) | (99) | (100) | (98) | (95) | (98) | (100) | |
| MPL | Mut. (%) | 0 | 3 | 0 | 0 | 2 | 0 | 2 | <0.0001 |
| | | (0) | (1) | (0) | (0) | (4) | (0) | (2) | |
| | WT (%) | 129 | 200 | 21 | 36 | 55 | 163 | 92 | |
| | | (100) | (99) | (100) | (100) | (96) | (100) | (98) | |
| NF1 | Mut. (%) | 4 | 14 | 0 | 0 | 1 | 19 | 7 | 0.0032 |
| | | (3) | (8) | (0) | (0) | (2) | (15) | (9) | |
| | WT (%) | 111 | 155 | 22 | 34 | 49 | 109 | 67 | |
| | | (97) | (92) | (100) | (100) | (98) | (85) | (91) | |
| NPM1 | Mut. (%) | 138 | 163 | 22 | 37 | 4 | 5 | 12 | <0.0001 |
| | | (91) | (70) | (100) | (90) | (7) | (3) | (10) | |
| | WT (%) | 13 | 71 | 0 | 4 | 57 | 185 | 104 | |
| | | (9) | (30) | (0) | (10) | (93) | (97 | (90) | |
| NRAS | Mut. (%) | 26 | 41 | 2 | 5 | 6 | 32 | 12 | 0.0083 |
| | | (17) | (17) | (9) | (12) | (10) | (17) | (10) | |
| | WT (%) | 127 | 196 | 20 | 37 | 57 | 161 | 106 | |
| | | (83) | (83) | (91) | (88) | (90) | (83) | (90) | |
| PHF6 | Mut. (%) | 1 | 7 | 0 | 0 | 7 | 9 | 5 | 0.0014 |
| | | (1) | (3) | (0) | (0) | (11) | (5) | (4) | |
| | WT (%) | 152 | 230 | 22 | 42 | 56 | 184 | 113 | |
| | | (99) | (97) | (100) | (100) | (89) | (95) | (96) | |
| PTEN | Mut. (%) | 1 | 1 | 0 | 0 | 1 | 1 | 1 | 0.9923 |
| | | (1) | (0) | (0) | (0) | (2) | (1) | (1) | |
| | WT (%) | 152 | 236 | 22 | 42 | 62 | 192 | 117 | |
| | | (99) | (100) | (100) | (100) | (98) | (99) | (99) | |
| PTPN11 | Mut. (%) | 17 | 40 | 2 | 5 | 4 | 17 | 2 | <0.0001 |
| | | (11) | (17) | (9) | (12) | (6) | (9) | (2) | |
| | WT (%) | 136 | 197 | 20 | 37 | 59 | 176 | 116 | |
| | | (89) | (83) | (91) | (88) | (94) | (91) | (98) | |
| RAD21 | Mut. (%) | 4 | 11 | 1 | 1 | 0 | 2 | 2 | 0.4146 |
| | | (3) | (5) | (5) | (2) | (0) | (1) | (2) | |
| | WT (%) | 149 | 226 | 21 | 41 | 63 | 191 | 116 | |
| | | (97) | (95) | (95) | (98) | (100) | (99) | (98) | |
| RUNX1 | Mut. (%) | 6 | 7 | 0 | 1 | 13 | 59 | 21 | <0.0001 |
| | | (4) | (3) | (0) | (2) | (21) | (31) | (18) | |
| | WT (%) | 147 | 230 | 22 | 41 | 50 | 134 | 97 | |
| | | (96) | (97) | (100) | (98) | (79) | (69) | (82) | |
| SF1 | Mut. (%) | 0 | 2 | 1 | 1 | 0 | 2 | 3 | 0.3272 |
| | | (0) | (1) | (5) | (2) | (0) | (1) | (3) | |
| | WT (%) | 153 | 235 | 21 | 41 | 63 | 191 | 115 | |
| | | (100) | (99) | (95) | (98) | (100) | (99) | (97) | |
| SF3A1 | Mut. (%) | 1 | 3 | 0 | 1 | 0 | 3 | 0 | 0.7524 |
| | | (1) | (1) | (0) | (2) | (0) | (2) | (0) | |
| | WT (%) | 152 | 234 | 22 | 41 | 63 | 190 | 118 | |
| | | (99) | (99) | (100) | (98) | (100) | (98) | (100) | |
| SF3B1 | Mut. (%) | 2 | 7 | 0 | 1 | 1 | 22 | 2 | <0.0001 |
| | | (1) | (3) | (0) | (2) | (2) | (11) | (2) | |
| | WT (%) | 151 | 230 | 22 | 41 | 62 | 171 | 116 | |
| | | (99) | (97) | (100) | (98) | (98) | (89) | (98) | |
| SFRS2 | Mut. (%) | 2 | 4 | 4 | 9 | 15 | 14 | 24 | <0.0001 |
| | | (1) | (2) | (18) | (21) | (24) | (7) | (21) | |
| | WT (%) | 150 | 232 | 18 | 33 | 48 | 178 | 92 | |
| | | (99) | (98) | (82) | (79) | (76) | (93) | (79) | |
| STAG2 | Mut. (%) | 2 | 7 | 0 | 1 | 4 | 5 | 13 | 0.0005 |
| | | (1) | (3) | (0) | (2) | (6) | (3) | (11) | |
| | WT (%) | 151 | 230 | 22 | 41 | 59 | 188 | 105 | |
| | | (99) | (97) | (100) | (98) | (94) | (97) | (89) | |
| TET2 | Mut. (%) | 21 | 35 | 17 | 2 | 5 | 22 | 23 | <0.0001 |
| | | (14) | (15) | (77) | (5) | (8) | (11) | (19) | |
| | WT (%) | 132 | 202 | 5 | 40 | 58 | 171 | 95 | |
| | | (86) | (85) | (23) | (95) | (92) | (89) | (81) | |
| TP53 | Mut. (%) | 3 | 5 | 0 | 0 | 6 | 46 | 21 | <0.0001 |
| | | (2) | (2) | (0) | (0) | (10) | (24) | (18) | |
| | WT (%) | 150 | 232 | 22 | 42 | 57 | 147 | 97 | |
| | | (98) | (98) | (100) | (100) | (90) | (76) | (82) | |
| U2AF1 | Mut. (%) | 0 | 3 | 0 | 0 | 7 | 15 | 8 | 0.0003 |
| | | (0) | (1) | (0) | (0) | (11) | (8) | (7) | |
| | WT (%) | 153 | 234 | 22 | 42 | 56 | 178 | 110 | |
| | | (100) | (99) | (100) | (100) | (89) | (92) | (93) | |
| WT1 | Mut. (%) | 5 | 42 | 0 | 0 | 2 | 24 | 5 | <0.0001 |
| | | (3) | (18) | (0) | (0) | (3) | (12) | (4) | |
| | WT (%) | 148 | 195 | 22 | 42 | 61 | 169 | 113 | |
| | | (97) | (82) | (100) | (100) | (97) | (88) | (96) | |
| ZRSR2 | Mut. (%) | 9 | 8 | 1 | 2 | 2 | 16 | 6 | 0.5255 |
| | | (6) | (3) | (5) | (5) | (3) | (8) | (5) | |
| | WT (%) | 144 | 229 | 21 | 40 | 61 | 177 | 112 | |
| | | (94) | (97) | (95) | (95) | (97) | (92) | (95) | |
| CEBPA-sm | Mut. (%) | 11 | 17 | 4 | 4 | 5 | 3 | 7 | 0.1745 |
| | | (8) | (9) | (20) | (11) | (8) | (2) | (9) | |
| | WT (%) | 126 | 179 | 16 | 33 | 54 | 162 | 75 | |
| | | (92) | (91) | (80) | (89) | (92) | (98) | (91) | |
| CEBPA-dm | Mut. (%) | 4 | 4 | 0 | 1 | 0 | 4 | 0 | <0.0001 |
| | | (3) | (2) | (0) | (3) | (0) | (2) | (0) | |
| | WT (%) | 133 | 192 | 20 | 36 | 59 | 161 | 82 | |
| | | (97) | (98) | (100) | (97) | (100) | (98) | (100) | |
| FLT3-ITD | Mut. (%) | 61 | 81 | 10 | 14 | 7 | 25 | 13 | <0.0001 |
| | | (40) | (34) | (45) | (33) | (11) | (13) | (11) | |
| | WT (%) | 92 | 156 | 12 | 28 | 56 | 168 | 105 | |
| | | (60) | (66) | (55) | (67) | (89) | (87) | (89) | |
| FLT3-TKD | Mut. (%) | 18 | 39 | 2 | 3 | 1 | 17 | 6 | 0.0001 |
| | | (12) | (17) | (9) | (7) | (2) | (9) | (5) | |
| | WT (%) | 134 | 194 | 20 | 39 | 60 | 173 | 110 | |
| | | (88) | (83) | (91) | (93) | (98) | (91) | (95) | |
| IDH2 p172 | Mut. (%) | 1 | 1 | 0 | 1 | 22 | 6 | 7 | <0.0001 |
| | | (1) | (0) | (0) | (2) | (35) | (3) | (6) | |
| | WT (%) | 152 | 236 | 22 | 41 | 41 | 187 | 111 | |
| | | (99) | (100) | (100) | (98) | (65) | (97) | (94) | |
| IDH2 p140 | Mut. (%) | 11 | 19 | 1 | 19 | 18 | 6 | 18 | <0.0001 |
| | | (7) | (8) | (5) | (45) | (29) | (3) | (15) | |
| | WT (%) | 142 | 218 | 21 | 23 | 45 | 187 | 100 | |
| | | (93) | (92) | (95) | (55) | (71) | (97) | (85) | |
| inv(3)/t(3; 3) | Pos. | 0 | 1 | 0 | 0 | 0 | 21 | 0 | <0.0001 |
| | | (0) | (0) | (0) | (0) | (0) | (11) | (0) | |
| | Neg. | 153 | 236 | 22 | 42 | 63 | 172 | 118 | |
| | | (100) | (100) | (100) | (100) | (100) | (89) | (100) | |
| t(9; 22) | Pos. | 0 | 0 | 0 | 0 | 1 | 6 | 6 | 0.0068 |
| | | (0) | (0) | (0) | (0) | (2) | (3) | (5) | |
| | Neg. | 153 | 237 | 22 | 42 | 62 | 187 | 112 | |
| | | (100) | (100) | (100) | (100) | (98) | (97) | (95) | |
| Monosomy | Pos. | 1 | 4 | 0 | 0 | 6 | 52 | 20 | <0.0001 |
| 5, del(5q) | | (1) | (2) | (0) | (0) | (10) | (27) | (17) | |
| | Neg. | 152 | 233 | 22 | 42 | 57 | 141 | 98 | |
| | | (99) | (98) | (100) | (100) | (90) | (73) | (83) | |
| monosomy | Pos. | 1 | 1 | 0 | 0 | 4 | 51 | 29 | <0.0001 |
| 7 | | (1) | (0) | (0) | (0) | (6) | (26) | (25) | |
| | Neg. | 152 | 236 | 22 | 42 | 59 | 142 | 89 | |
| | | (99) | (100) | (100) | (100) | (94) | (74) | (75) | |
| del(7q) | Pos. | 2 | 2 | 0 | 0 | 8 | 25 | 11 | <0.0001 |
| | | (1) | (1) | (0) | (0) | (13) | (13) | (9) | |
| | Neg. | 151 | 235 | 22 | 42 | 55 | 168 | 107 | |
| | | (99) | (99) | (100) | (100) | (87 | (87) | (91) | |
| Abnormal | Pos | 2 | 0 | 0 | 0 | 0 | 2 | 1 | 0.2214 |
| chr. 7 | | (1) | (0) | (0) | (0) | (0) | (1) | (1) | |
| (other) | Neg. | 151 | 237 | 22 | 42 | 63 | 191 | 117 | |
| | | (99) | (100) | (100) | (100) | (100) | (99) | (99) | |
| Plus 8, +8q | Pos. | 10 | 20 | 0 | 1 | 14 | 25 | 20 | <0.0001 |
| | | (7) | (8) | (0) | (2) | (22) | (13) | (17) | |
| | Neg. | 143 | 217 | 22 | 41 | 49 | 168 | 98 | |
| | | (93) | (92) | (100) | (98) | (78) | (87) | (83) | |
| del(9q) | Pos. | 7 | 3 | 0 | 0 | 0 | 0 | 3 | 0.0571 |
| | | (5) | (1) | (0) | (0) | (0) | (0) | (3) | |
| | Neg. | 146 | 234 | 22 | 42 | 63 | 193 | 115 | |
| | | (95) | (99) | (100) | (100) | (100) | (100) | (97) | |
| Abnormal | Pos. | 0 | 1 | 0 | 0 | 3 | 21 | 9 | <0.0001 |
| chr. 12 | | (0) | (0) | (0) | (0) | (5) | (11) | (8) | |
| | Neg. | 153 | 236 | 22 | 42 | 60 | 172 | 109 | |
| | | (100) | (100) | (100) | (100) | (95) | (89) | (92) | |
| Plus 13 | Pos. | 1 | 1 | 0 | 0 | 2 | 8 | 10 | 0.0003 |
| | | (1) | (0) | (0) | (0) | (3) | (4) | (8) | |
| | Neg. | 152 | 236 | 22 | 42 | 61 | 185 | 108 | |
| | | (99) | (100) | (100) | (100) | (97) | (96) | (92) | |
| Monosomy | Pos. | 4 | 2 | 0 | 0 | 4 | 43 | 14 | <0.0001 |
| 17, | | (3) | (1) | (0) | (0) | (6) | (22) | (12) | |
| abnormal | Neg. | 149 | 235 | 22 | 42 | 59 | 150 | 104 | |
| chr. 17p | | (97) | (99) | (100) | (100) | (94) | (78) | (88) | |
| Monosomy | Pos | 1 | 0 | 0 | 0 | 3 | 18 | 5 | <0.0001 |
| | | (1) | (0) | (0) | (0) | (5) | (9) | (4) | |
| 18, del(18q) | Neg. | 152 | 237 | 22 | 42 | 60 | 175 | 113 | |
| | | (99) | (100) | (100) | (100) | (95) | (91) | (96) | |
| Monosomy | Pos. | 2 | 2 | 0 | 0 | 2 | 15 | 6 | 0.0014 |
| 20, del(20q) | | (1) | (1) | (0) | (0) | (3) | (8) | (5) | |
| | Neg. | 151 | 235 | 22 | 42 | 61 | 178 | 112 | |
| | | (99) | (99) | (100) | (100) | (97) | (92) | (95) | |
| Plus 21 | Pos | 2 | 2 | 0 | 0 | 4 | 8 | 4 | 0.1082 |
| | | (1) | (1) | (0) | (0) | (6) | (4) | (3) | |
| | Neg. | 151 | 235 | 22 | 42 | 59 | 185 | 114 | |
| | | (99) | (99) | (100) | (100) | (94) | (96) | (97) | |
| Plus 22 | Pos. | 1 | 0 | 0 | 0 | 2 | 12 | 4 | <0.0001 |
| | | (1) | (0) | (0) | (0) | (3) | (6) | (3) | |
| | Neg. | 152 | 237 | 22 | 42 | 61 | 181 | 114 | |
| | | (99) | (100) | (100) | (100) | (97) | (94) | (97) | |
| Minus Y | Pos. | 0 | 2 | 0 | 0 | 1 | 5 | 7 | <0.0001 |
| | | (0) | (1) | (0) | (0) | (2) | (3) | (6) | |
| | Neg. | 153 | 235 | 22 | 42 | 62 | 188 | 111 | |
| | | (100) | (99) | (100) | (100) | (98) | (97) | (94) | |
| t(8; 21) | Pos. | 0 | 0 | 0 | 0 | 0 | 0 | 0 | <0.0001 |
| | | (0) | (0) | (0) | (0) | (0) | (0) | (0) | |
| | Neg. | 153 | 237 | 22 | 42 | 63 | 193 | 118 | |
| | | (100) | (100) | (100) | (100) | (100) | (100) | (100) | |
| inv(16) | Pos. | 0 | 0 | 0 | 0 | 0 | 0 | 0 | <0.0001 |
| | | (0) | (0) | (0) | (0) | (0) | (0) | (0) | |
| | Neg. | 153 | 237 | 22 | 42 | 63 | 193 | 118 | |
| | | (100) | (100) | (100) | (100) | (100) | (100) | (100) | |
| t(6; 9) | Pos. | 0 | 6 | 0 | 0 | 0 | 0 | 0 | 0.0945 |
| | | (0) | (3) | (0) | (0) | (0) | (0) | (0) | |
| | Neg. | 153 | 231 | 22 | 42 | 63 | 193 | 118 | |
| | | (100) | (97) | (100) | (100) | (100) | (100) | (100) | |
| Plus 11, +11q | Pos. | 0 | 3 | 0 | 0 | 5 | 13 | 7 | 0.0001 |
| | | (0) | (1) | (0) | (0) | (8) | (7) | (6) | |
| | Neg. | 153 | 234 | 22 | 42 | 58 | 180 | 111 | |
| | | (100) | (99) | (100) | (100) | (92) | (93) | (94) | |
| Abnormal | Pos. | 0 | 1 | 0 | 0 | 0 | 9 | 5 | 0.0066 |
| chr. 4 | | (0) | (0) | (0) | (0) | (0) | (5) | (4) | |
| | Neg. | 153 | 236 | 22 | 42 | 63 | 184 | 113 | |
| | | (100) | (100) | (100) | (100) | (100) | (95) | (96) | |
| Complex | Pos. | 5 | 7 | 0 | 1 | 9 | 62 | 30 | <0.0001 |
| karyotype | | (3) | (3) | (0) | (2) | (14) | (32) | (25) | |
| | Neg. | 148 | 230 | 22 | 41 | 54 | 131 | 88 | |
| | | (97) | (97) | (100) | (98) | (86) | (68) | (75) | |
| t(9; 11) | Pos. | 0 | 0 | 0 | 0 | 0 | 1 | 1 | <0.0001 |
| | | (0) | (0) | (0) | (0) | (0) | (1) | (1) | |
| | Neg. | 153 | 237 | 22 | 42 | 63 | 192 | 117 | |
| | | (100) | (100) | (100) | (100) | (100) | (99) | (99) | |
| t(v; 11) | Pos. | 1 | 6 | 0 | 0 | 0 | 2 | 0 | <0.0001 |
| (other) | | (1) | (3) | (0) | (0) | (0) | (1) | (0) | |
| | Neg. | 152 | 231 | 22 | 42 | 63 | 191 | 118 | |
| | | (99) | (97) | (100) | (100) | (100) | (99) | (100) | |

TABLE 3
Summary of clinical characteristics of AML patients separated by epitype

| | Epitype 2 | | Epitype 3 | | Epitype 4 | | Epitype 5 | | Epitype 6 | | Epitype 7 | | Epitype 8 | |
|---|---|---|---|---|---|---|---|---|---|---|---|---|---|---|
| Characteristic | n = 29 | | n = 26 | | n = 74 | | n = 80 | | n = 38 | | n = 144 | | n = 224 | |
| Age (years) | | | | | | | | | | | | | | |
| Median | 36 | | 40 | | 39 | | 52 | | 40 | | 51 | | 49 | |
| Range | 23-72 | | 19-74 | | 17-68 | | 17-78 | | 19-84 | | 18-84 | | 17-81 | |
| Age group, no. (%) | | | | | | | | | | | | | | |
| Younger | 27 | (93) | 21 | (81) | 70 | (95) | 57 | (71) | 31 | (82) | 114 | (79) | 190 | (85) |
| Older | 2 | (7) | 5 | (19) | 4 | (5) | 23 | (29) | 7 | (18) | 30 | (21) | 34 | (15) |
| Sex, no. (%) | | | | | | | | | | | | | | |
| Male | 17 | (59) | 12 | (46) | 47 | (64) | 39 | (49) | 20 | (53) | 76 | (53) | 105 | (47) |
| Female | 12 | (41) | 14 | (54) | 27 | (36) | 41 | (51) | 18 | (47) | 68 | (47) | 119 | (53) |
| Race, no. (%) | | | | | | | | | | | | | | |
| White | 21 | (72) | 20 | (80) | 61 | (82) | 67 | (86) | 27 | (73) | 128 | (89) | 203 | (91) |
| Non-white | 8 | (28) | 5 | (20) | 13 | (18) | 11 | (14) | 10 | (27) | 16 | (11) | 19 | (9) |
| Hemoglobin (g/dL) | 1 unknown | | 1 unknown | | 1 unknown | | 3 unknown | | 1 unknown | | 5 unknown | | 6 unknown | |
| Median | 9.2 | | 9.1 | | 9.5 | | 9.5 | | 9.3 | | 9.4 | | 9.0 | |
| Range | 2.9-12.6 | | 5.5-11.6 | | 4.9-13.4 | | 6.2-14.7 | | 5.7-14.8 | | 2.3-13.7 | | 4.2-14.0 | |
| Platelet count (×10⁹/L) | | | | | | | 2 unknown | | 1 unknown | | | | 6 unknown | |
| Median | 37 | | 37 | | 39 | | 59 | | 43 | | 61 | | 56 | |
| Range | 7-102 | | 7-191 | | 10-266 | | 8-242 | | 8-137 | | 8-387 | | 12-648 | |
| WBC count (×10⁹/L) | | | | | | | 1 unknown | | 1 unknown | | | | 4 unknown | |
| Median | 14.0 | | 33.8 | | 23.5 | | 43.5 | | 40.8 | | 41.0 | | 25.9 | |
| Range | 1.8-257.0 | | 0.4-244.3 | | 2.2-295.0 | | 1.1-149.1 | | 1.1-268.0 | | 0.9-308.8 | | 0.8-355.0 | |
| % Blood Blasts | | | | | | | | | 1 unknown | | | | 1 unknown | |
| Median | 70 | | 42 | | 76 | | 57 | | 66 | | 61 | | 54 | |
| Range | 12-90 | | 5-85 | | 7-98 | | 0-95 | | 0-99 | | 0-95 | | 0-97 | |
| % Bone Marrow Blasts | | | | | | | | | 1 unknown | | | | 2 unknown | |
| Median | 54 | | 48 | | 68 | | 80 | | 80 | | 74 | | 64 | |
| Range | 24-87 | | 24-79 | | 19-93 | | 6-96 | | 38-97 | | 22-97 | | 0-95 | |
| Extramedullary | 6 | (21) | 8 | (33) | 18 | (24) | 19 | (26) | 16 | (42) | 52 | (38) | 60 | (29) |

Involvement, no. (%)

TABLE 3
Summary of clinical characteristics of AML patients separated by epitype

| | Epitype 9 | | Epitype 10 | | Epitype 11 | | Epitype 12 | | Epitype 13 | | |
|---|---|---|---|---|---|---|---|---|---|---|---|
| Characteristic | n = 22 | | n = 40 | | n = 55 | | n = 180 | | n = 109 | | P-value |
| Age (years) | | | | | | | | | | | <001 |
| Median | 64 | | 58 | | 55 | | 57 | | 61 | | |
| Range | 46-79 | | 21-83 | | 17-82 | | 19-82 | | 21-85 | | |
| Age group, no. (%) | | | | | | | | | | | <001 |
| Younger | 9 | (41) | 20 | (50) | 33 | (60) | 101 | (56) | 52 | (48) | |
| Older | 13 | (59) | 20 | (50) | 22 | (40) | 79 | (44) | 57 | (52) | |
| Sex, no. (%) | | | | | | | | | | | .03 |
| Male | 11 | (50) | 24 | (60) | 32 | (58) | 103 | (57) | 76 | (70) | |
| Female | 11 | (50) | 16 | (40) | 23 | (42) | 77 | (43) | 33 | (30) | |
| Race, no. (%) | | | | | | | | | | | .006 |
| White | 21 | (95) | 37 | (95) | 46 | (85) | 158 | (90) | 97 | (92) | |
| Non-white | 1 | (5) | 2 | (5) | 8 | (15) | 18 | (10) | 9 | (8) | |
| Hemoglobin (g/dL) | | | | | 2 unknown | | 6 unknown | | 8 unknown | | .02 |
| Median | 9.9 | | 9.6 | | 8.9 | | 9.0 | | 9.2 | | |
| Range | 5.4-15.0 | | 5.6-14.1 | | 5.0-12.8 | | 4.6-14.4 | | 3.0-25.1 | | |
| Platelet count (×10⁹/L) | | | | | 2 unknown | | 3 unknown | | 5 unknown | | <.001 |
| Median | 56 | | 65 | | 81 | | 60 | | 65 | | |
| Range | 20-433 | | 11-850 | | 6-245 | | 4-317 | | 4-266 | | |
| WBC count (×10⁹/L) | | | | | l unknown | | 2 unknown | | 5 unknown | | <.001 |
| Median | 41.9 | | 41.4 | | 12.9 | | 9.7 | | 13.2 | | |
| Range | 2.8-450.0 | | 1.5-343.6 | | 0.7-248.0 | | 0.6-203.8 | | 1.1-434.1 | | |
| % Blood Blasts | | | | | | | 1 unknown | | 3 unknown | | <001 |
| Median | 80 | | 82 | | 70 | | 40 | | 27 | | |
| Range | 17-98 | | 8-99 | | 0-99 | | 0-97 | | 0-96 | | |
| % Bone Marrow Blasts | | | | | | | 2 unknown | | 2 unknown | | <001 |
| Median | 84 | | 81 | | 78 | | 57 | | 48 | | |
| Range | 38-99 | | 0-94 | | 25-94 | | 12-96 | | 16-95 | | |
| Extramedullary | 4 | (19) | 10 | (27) | 10 | (19) | 30 | (17) | 19 | (19) | .003 |

Involvement, no. (%)

TABLE 4
Summary of outcome characteristics of AML patients separated by epitype

| | Subtype 2 | | Subtype 3 | | Subtype 4 | | Subtype 5 | |
|---|---|---|---|---|---|---|---|---|
| Characteristic | n = 29 | | n = 26 | | n = 74 | | n = 80 | |
| Early Death, no (%) | 0 | (0) | 0 | (0) | 0 | (0) | 0 | (0) |
| CR, no.(%) | 24 | (83) | 23 | (88) | 69 | (93) | 56 | (70) |
| Death in CR, no.(%) | 2 | (8) | 2 | (9) | 3 | (4) | 9 | (16) |
| Relapse Rate, no.(%) | 11 | (46) | 14 | (61) | 30 | (43) | 34 | (61) |
| Number Expired, no.(%) | 12 | (41) | 12 | (46) | 32 | (43) | 65 | (81) |
| Disease-Free Survival (DFS) | | | | | | | | |
| Median (years) | 7.1 | | 1.7 | | NR | | 0.7 | |
| % Disease-free at 1 year | 63 | (40-78) | 61 | (38-77) | 68 | (56-78) | 45 | (31-57) |
| % Disease-free at 3 years | 50 | (29-68) | 39 | (20-58) | 55 | (43-66) | 25 | (15-37) |
| % Disease-free at 5 years | 50 | (29-68) | 35 | (17-54) | 52 | (40-63) | 25 | (15-37) |
| Overall Survival (OS) | | | | | | | | |
| Median (years) | 13.3 | | NR | | NR | | 0.8 | |
| % Alive at 1 year | 86 | (67-95) | 88 | (68-96) | 88 | (78-93) | 44 | (33-54) |
| % Alive at 3 years | 66 | (45-80) | 62 | (40-77) | 68 | (56-77) | 25 | (16-35) |
| % Alive at 5 years | 66 | (45-80) | 58 | (37-74) | 61 | (49-71) | 21 | (13-31) |

TABLE 4
Summary of outcome characteristics of AML patients separated by epitype

| | Subtype 6 | | Subtype 7 | | Subtype 8 | | Subtype 9 | |
|---|---|---|---|---|---|---|---|---|
| Characteristic | n = 38 | | n = 144 | | n = 224 | | n = 22 | |
| Early Death, no (%) | 0 | (0) | 0 | (0) | 0 | (0) | 0 | (0) |
| CR, no.(%) | 25 | (66) | 122 | (85) | 166 | (74) | 17 | (77) |
| Death in CR, no.(%) | 0 | (0) | 14 | (11) | 19 | (11) | 2 | (12) |
| Relapse Rate, no.(%) | 23 | (92) | 75 | (61) | 108 | (65) | 12 | (71) |
| Number Expired, no.(%) | 34 | (89) | 109 | (76) | 153 | (71) | 16 | (73) |
| Disease-Free Survival (DFS) | | | | | | | | |
| Median (years) | 0.7 | | 0.9 | | 1.2 | | 1.1 | |
| % Disease-free at 1 year | 16 | (5-33) | 46 | (37-54) | 53 | (45-60) | 59 | (33-78) |
| % Disease-free at 3 years | 8 | (1-22) | 32 | (24-41) | 33 | (26-40) | 29 | (11-51) |
| % Disease-free at 5 years | 8 | (1-22) | 32 | (23-40) | 30 | (23-27) | 29 | (11-51) |
| Overall Survival (OS) | | | | | | | | |
| Median (years) | 1.0 | | 1.2 | | 1.5 | | 1.8 | |
| % Alive at 1 year | 53 | (36-67) | 57 | (49-65) | 61 | (54-67) | 59 | (36-76) |
| % Alive at 3 years | 13 | (5-26) | 32 | (25-40) | 37 | (31-43) | 41 | (21-60) |
| % Alive at 5 years | 13 | (5-26) | 28 | (21-36) | 34 | (28-40) | 36 | (17-56) |

| | Subtype 10 | | Subtype 11 | | Subtype 12 | | Subtype 13 | | |
|---|---|---|---|---|---|---|---|---|---|
| Characteristic | n = 40 | | n = 55 | | n = 180 | | n = 109 | | P-value |
| Early Death, no (%) | 0 | (0) | 0 | (0) | 0 | (0) | 2 | (2) | .11 |
| CR, no.(%) | 31 | (78) | 39 | (71) | 65 | (36) | 39 | (36) | <.001 |
| Death in CR, no.(%) | 3 | (10) | 3 | (8) | 3 | (5) | 8 | (21) | .16 |
| Relapse Rate, no.(%) | 19 | (61) | 31 | (79) | 61 | (94) | 27 | (69) | <.001 |
| Number Expired, no.(%) | 25 | (63) | 45 | (82) | 172 | (96) | 99 | (91) | <.001 |
| Disease-Free Survival (DFS) | | | | | | | | | <.001 |
| Median (years) | 1.8 | | 1.3 | | 0.7 | | 0.7 | | |
| % Disease-free at 1 year | 71 | (52-84) | 59 | (42-72) | 32 | (21-44) | 36 | (21-51) | |
| % Disease-free at 3 years | 39 | (22-55) | 21 | (10-34) | 8 | (3-16) | 13 | (5-25) | |
| % Disease-free at 5 years | 35 | (19-52) | 13 | (5-25) | 2 | (0-7) | 10 | (3-22) | |
| Overall Survival (OS) | | | | | | | | | <.001 |
| Median (years) | 3.1 | | 1.3 | | 0.7 | | 0.8 | | |
| % Alive at 1 year | 83 | (67-91) | 62 | (48-73) | 41 | (33-48) | 40 | (31-49) | |
| % Alive at 3 years | 50 | (34-64) | 31 | (19-43) | 14 | (9-19) | 16 | (10-23) | |
| % Alive at 5 years | 45 | (29-59) | 22 | (12-33) | 8 | (4-12) | 9 | (5-15) | |

TABLE 5
Individual features assessed using
multistage random effects modeling

| Feature name | Feature class |
|---|---|
| Bone Marrow Blast count | Clinical |
| ECOG Performance status | Clinical |
| Hemoglobin | Clinical |
| LDH | Clinical |
| Peripheral Blood Blast count | Clinical |
| platelets | Clinical |
| Splenomegaly | Clinical |
| tAML | Clinical |
| WBC | Clinical |
| abnormal chr 12 (monosomy 12, del(12p), | CNA |
| abnormal 12p) | |
| abnormal chr 17 (monosomy 17, del(17p), | CNA |
| abnormal 17p) | |
| abnormal chr 3q | CNA |
| abnormal chr 4 (monosomy 4, del(4p), | CNA |
| abnormal 4p) | |
| abnormal chr 7 (other) | CNA |
| complex karyotype | CNA |
| del(7q) | CNA |
| del(9q) | CNA |
| minusY | CNA |
| monosomy 18, del(18q) | CNA |
| monosomy 20, del(20q) | CNA |
| monosomy 5, del(5q) | CNA |
| monosomy 7 | CNA |
| plus11, +11q | CNA |
| plus13 | CNA |
| plus21 | CNA |
| plus22 | CNA |
| plus8, +8q | CNA |
| Age of diagnosis | Demographics |
| Gender | Demographics |
| E10 | DNA methylation |
| E11 | DNA methylation |
| E12 | DNA methylation |
| E13 | DNA methylation |
| E2 | DNA methylation |
| E3 | DNA methylation |
| E4 | DNA methylation |
| E5 | DNA methylation |
| E6 | DNA methylation |
| E7 | DNA methylation |
| E8 | DNA methylation |
| E9 | DNA methylation |
| SHS | DNA methylation |
| inv(16) | Fusions |
| t(6; 9) | Fusions |
| t(8; 21) | Fusions |
| t(9; 11) | Fusions |
| t(v; 11) | Fusions |
| ASXL1 | SNV/Indel |
| BCOR | SNV/Indel |
| BRAF | SNV/Indel |
| CBL | SNV/Indel |
| CEBPA-dm | SNV/Indel |
| CEBPA-sm | SNV/Indel |
| DNMT3A | SNV/Indel |
| ETV6 | SNV/Indel |
| EZH2 | SNV/Indel |
| FLT3-ITD | SNV/Indel |
| FLT3-TKD | SNV/Indel |
| GATA2 | SNV/Indel |
| IDH1 | SNV/Indel |
| IDH2 p140 | SNV/Indel |
| IDH2 p172 | SNV/Indel |
| IKZF1 | SNV/Indel |
| JAK2 | SNV/Indel |
| KIT | SNV/Indel |
| KRAS | SNV/Indel |
| MLL | SNV/Indel |
| MPL | SNV/Indel |
| NF1 | SNV/Indel |
| NPM1 | SNV/Indel |
| NRAS | SNV/Indel |
| PHF6 | SNV/Indel |
| PTEN | SNV/Indel |
| PTPN11 | SNV/Indel |
| RAD21 | SNV/Indel |
| RUNX1 | SNV/Indel |
| SF1 | SNV/Indel |
| SF3A1 | SNV/Indel |
| SF3B1 | SNV/Indel |
| SRFS2 | SNV/Indel |
| STAG2 | SNV/Indel |
| TET2 | SNV/Indel |
| TP53 | SNV/Indel |
| U2AF1 | SNV/Indel |
| WT1 | SNV/Indel |
| ZRSR2 | SNV/Indel |

TABLE 6
Association of features with overall survival using multistage random effects modelling

| | | beta | | | | | | | |
|---|---|---|---|---|---|---|---|---|---|
| | | (log- | hazard | | | sd | | Q-value | Q-value |
| Feature name | Feature class | hazard) | exp(beta) | n | sd | (var) | P-value | (B-Y) | (B-H) |
| Age of diagnosis | Demographics | 0.2169 | 1.2422 | 1021 | 0.0268 | 0.0281 | 0 | 0 | 0 |
| E4 | DNA methylation | −0.8166 | 0.4419 | 74 | 0.1943 | 0.256 | 0 | 0.0038 | 0.0007 |
| E12 | DNA methylation | 0.3695 | 1.4471 | 180 | 0.0857 | 0.1366 | 0 | 0.0029 | 0.0005 |
| NPM1 | SNV/Indel | −0.3656 | 0.6938 | 382 | 0.0769 | 0.1299 | 0 | 0.0005 | 0.0001 |
| SHS | DNA methylation | 0.344 | 1.4105 | 213 | 0.0832 | 0.1186 | 0 | 0.0043 | 0.0008 |
| WBC | Clinical | 0.2746 | 1.316 | 1007 | 0.0575 | 0.0639 | 0 | 0.0005 | 0.0001 |
| abnormal chr 17 | CNA | 0.425 | 1.5295 | 63 | 0.1135 | 0.1688 | 0.0002 | 0.0188 | 0.0034 |
| (monosomy 17 or | | | | | | | | | |
| del(17p) or abnormal | | | | | | | | | |
| 17p) | | | | | | | | | |
| E6 | DNA methylation | 0.7105 | 2.035 | 38 | 0.1986 | 0.239 | 0.0003 | 0.0302 | 0.0055 |
| monosomy 5 or del(5q) | CNA | 0.3989 | 1.4901 | 83 | 0.113 | 0.1583 | 0.0004 | 0.0302 | 0.0055 |
| monosomy 7 | CNA | 0.3774 | 1.4585 | 83 | 0.1064 | 0.1315 | 0.0004 | 0.0302 | 0.0055 |
| SHS: FLT3_ITD | Gene: DNA methylation | 0.2618 | 1.2992 | 112 | 0.0775 | 0.1318 | 0.0007 | 0.0484 | 0.0089 |
| | interaction | | | | | | | | |
| t(9; 11) | Fusions | −0.8056 | 0.4468 | 24 | 0.2475 | 0.277 | 0.0011 | 0.0688 | 0.0126 |
| E13 | DNA methylation | 0.2975 | 1.3466 | 109 | 0.0942 | 0.1439 | 0.0016 | 0.0888 | 0.0162 |
| plus11 or +11q | CNA | 0.382 | 1.4652 | 26 | 0.1236 | 0.178 | 0.002 | 0.1039 | 0.019 |
| E8: NPM1 | Gene: DNA methylation | −0.21 | 0.8106 | 156 | 0.0688 | 0.1331 | 0.0023 | 0.1095 | 0.02 |
| | interaction | | | | | | | | |
| NPM1: WT1 | Gene: Gene interaction | 0.2062 | 1.229 | 36 | 0.0695 | 0.1493 | 0.003 | 0.138 | 0.0252 |
| LDH | Clinical | 0.149 | 1.1607 | 759 | 0.0531 | 0.1607 | 0.005 | 0.2161 | 0.0395 |
| IDH1 | SNV/Indel | 0.2277 | 1.2557 | 88 | 0.0828 | 0.1333 | 0.006 | 0.2416 | 0.0442 |
| FLT3-ITD | SNV/Indel | 0.2185 | 1.2442 | 223 | 0.0819 | 0.1164 | 0.0077 | 0.2938 | 0.0537 |
| NPM1: FLT3-TKD | Gene: Gene interaction | −0.1773 | 0.8376 | 60 | 0.0675 | 0.1434 | 0.0087 | 0.3159 | 0.0577 |
| SF3A1 | SNV/Indel | 0.1767 | 1.1932 | 9 | 0.0718 | 0.1854 | 0.0138 | 0.4666 | 0.0853 |
| E2 | DNA methylation | −0.6267 | 0.5343 | 29 | 0.2553 | 0.361 | 0.0141 | 0.4666 | 0.0853 |
| NRAS | SNV/Indel | 0.2015 | 1.2232 | 167 | 0.0826 | 0.1091 | 0.0147 | 0.4666 | 0.0853 |
| Splenomegaly | Clinical | 0.057 | 1.0586 | 69 | 0.0238 | 0.1699 | 0.0169 | 0.4917 | 0.0899 |
| tAML | Clinical | 0.057 | 1.0586 | 7 | 0.0238 | 0.1699 | 0.0169 | 0.4917 | 0.0899 |
| plus22 | CNA | −0.2788 | 0.7567 | 25 | 0.1188 | 0.1707 | 0.019 | 0.5316 | 0.0972 |
| E5 | DNA methylation | 0.3328 | 1.3949 | 80 | 0.1457 | 0.1861 | 0.0224 | 0.6022 | 0.1101 |
| Gender | Demographics | 0.1248 | 1.1329 | 1021 | 0.0549 | 0.0663 | 0.0232 | 0.6022 | 0.1101 |
| SHS: NPM1 | Gene: DNA methylation | 0.1645 | 1.1788 | 117 | 0.0735 | 0.1338 | 0.0251 | 0.6309 | 0.1153 |
| | interaction | | | | | | | | |
| E11: NPM1 | Gene: DNA methylation | −0.114 | 0.8923 | 36 | 0.0537 | 0.1539 | 0.0337 | 0.7855 | 0.1436 |
| | interaction | | | | | | | | |
| MLL | SNV/Indel | 0.1822 | 1.1999 | 14 | 0.0861 | 0.1723 | 0.0344 | 0.7855 | 0.1436 |
| WT1: FLT3-ITD | Gene: Gene interaction | 0.1517 | 1.1638 | 31 | 0.0718 | 0.148 | 0.0345 | 0.7855 | 0.1436 |
| SRFS2 | SNV/Indel | 0.1844 | 1.2024 | 74 | 0.0884 | 0.1353 | 0.037 | 0.8166 | 0.1492 |
| E8 | DNA methylation | 0.1546 | 1.1672 | 224 | 0.0749 | 0.1444 | 0.039 | 0.8205 | 0.15 |
| complex karyotype | CNA | 0.2187 | 1.2444 | 121 | 0.1062 | 0.1447 | 0.0395 | 0.8205 | 0.15 |
| SF1 | SNV/Indel | −0.1797 | 0.8355 | 9 | 0.0913 | 0.1659 | 0.0491 | 0.9462 | 0.1729 |
| SF3B1 | SNV/Indel | 0.1961 | 1.2166 | 37 | 0.0998 | 0.1454 | 0.0493 | 0.9462 | 0.1729 |
| E8: FLT3_ITD | Gene: DNA methylation | 0.1495 | 1.1612 | 77 | 0.0761 | 0.1347 | 0.0494 | 0.9462 | 0.1729 |
| | interaction | | | | | | | | |
| ECOG Performance | Clinical | 0.0917 | 1.0961 | 880 | 0.0471 | 0.0504 | 0.0515 | 0.9609 | 0.1756 |
| status | | | | | | | | | |
| abnormal chr 7 (other) | CNA | 0.236 | 1.2661 | 12 | 0.1222 | 0.2129 | 0.0535 | 0.9731 | 0.1778 |
| TP53 | SNV/Indel | 0.1859 | 1.2043 | 88 | 0.0974 | 0.1342 | 0.0563 | 0.9985 | 0.1825 |
| Platelets | Clinical | −0.0974 | 0.9072 | 1002 | 0.0526 | 0.0572 | 0.0643 | 1 | 0.2035 |
| abnormal chr 12 | CNA | 0.2182 | 1.2439 | 40 | 0.1203 | 0.1567 | 0.0696 | 1 | 0.2154 |
| (monosomy 12 or | | | | | | | | | |
| del(12p) or abnormal | | | | | | | | | |
| 12p) | | | | | | | | | |
| PB Blasts | Clinical | 0.0284 | 1.0288 | 1015 | 0.0161 | 0.0167 | 0.078 | 1 | 0.2357 |
| plus8 or +8q | CNA | 0.1708 | 1.1863 | 104 | 0.1 | 0.116 | 0.0875 | 1 | 0.2586 |
| monosomy 18 or del(18q) | CNA | 0.2119 | 1.236 | 23 | 0.125 | 0.179 | 0.0899 | 1 | 0.26 |
| U2AF1 | SNV/Indel | 0.1573 | 1.1704 | 40 | 0.0984 | 0.1383 | 0.1098 | 1 | 0.3057 |
| del(9q) | CNA | 0.2 | 1.2214 | 15 | 0.1252 | 0.1958 | 0.1103 | 1 | 0.3057 |
| STAG2 | SNV/Indel | 0.1541 | 1.1666 | 30 | 0.0975 | 0.1465 | 0.1139 | 1 | 0.3091 |
| del(7q) | CNA | 0.1745 | 1.1906 | 53 | 0.1135 | 0.1454 | 0.124 | 1 | 0.3299 |
| t(v; 11) | Fusions | 0.2885 | 1.3345 | 39 | 0.1928 | 0.2088 | 0.1346 | 1 | 0.3447 |
| E8: WT1 | Gene: DNA methylation | 0.1058 | 1.1116 | 40 | 0.0707 | 0.1463 | 0.1348 | 1 | 0.3447 |
| | interaction | | | | | | | | |
| WT1 | SNV/Indel | 0.1243 | 1.1324 | 86 | 0.0878 | 0.1309 | 0.1567 | 1 | 0.3917 |
| E3 | DNA methylation | −0.3383 | 0.713 | 26 | 0.2403 | 0.4114 | 0.1592 | 1 | 0.3917 |
| MPL | SNV/Indel | 0.1169 | 1.124 | 11 | 0.0836 | 0.1759 | 0.162 | 1 | 0.3917 |
| TET2 | SNV/Indel | 0.1103 | 1.1167 | 137 | 0.0819 | 0.1135 | 0.1777 | 1 | 0.422 |
| NF1 | SNV/Indel | 0.1258 | 1.1341 | 47 | 0.0944 | 0.1394 | 0.1827 | 1 | 0.4263 |
| BCOR | SNV/Indel | 0.1221 | 1.1298 | 63 | 0.0939 | 0.1211 | 0.1939 | 1 | 0.4446 |
| PTPN11 | SNV/Indel | 0.1124 | 1.119 | 101 | 0.0874 | 0.1293 | 0.1982 | 1 | 0.4469 |
| DNMT3A: IDH1 | Gene: Gene interaction | 0.0938 | 1.0984 | 42 | 0.0738 | 0.1419 | 0.2033 | 1 | 0.4497 |
| NRAS: TET2 | Gene: Gene interaction | −0.0966 | 0.9079 | 25 | 0.0764 | 0.1453 | 0.2062 | 1 | 0.4497 |
| IDH2 p140 | SNV/Indel | −0.0914 | 0.9127 | 91 | 0.0806 | 0.1319 | 0.2571 | 1 | 0.5515 |
| E7 | DNA methylation | 0.0803 | 1.0836 | 144 | 0.0723 | 0.1589 | 0.2665 | 1 | 0.5515 |
| abnormal chr 4 | CNA | 0.1346 | 1.1441 | 17 | 0.1224 | 0.1911 | 0.2716 | 1 | 0.5515 |
| (monosomy 4 or del(4p) | | | | | | | | | |
| or abnormal 4p) | | | | | | | | | |
| PTEN | SNV/Indel | 0.0745 | 1.0774 | 5 | 0.068 | 0.1866 | 0.2733 | 1 | 0.5515 |
| ASXL1: RUNX1 | Gene: Gene interaction | −0.0793 | 0.9238 | 25 | 0.0724 | 0.1471 | 0.2737 | 1 | 0.5515 |
| DNMT3A: TET2 | Gene: Gene interaction | 0.0811 | 1.0845 | 31 | 0.0763 | 0.1442 | 0.2883 | 1 | 0.5684 |
| minusY | CNA | 0.1329 | 1.1422 | 35 | 0.1265 | 0.1751 | 0.2934 | 1 | 0.5684 |
| SHS: E8 | DNA methylation: DNA | 0.0761 | 1.0791 | 71 | 0.0728 | 0.1373 | 0.2956 | 1 | 0.5684 |
| | methylation | | | | | | | | |
| DNMT3A: PTPN11 | Gene: Gene interaction | −0.0733 | 0.9293 | 34 | 0.0706 | 0.1482 | 0.2993 | 1 | 0.5684 |
| E9 | DNA methylation | −0.0852 | 0.9183 | 22 | 0.0828 | 0.1682 | 0.3034 | 1 | 0.5684 |
| NPM1: NRAS | Gene: Gene interaction | −0.0752 | 0.9276 | 67 | 0.0753 | 0.137 | 0.3183 | 1 | 0.5801 |
| DNMT3A: NRAS | Gene: Gene interaction | 0.0765 | 1.0795 | 47 | 0.0767 | 0.1414 | 0.3184 | 1 | 0.5801 |
| SRFS2: IDH2_p140 | Gene: Gene interaction | −0.0685 | 0.9337 | 29 | 0.0699 | 0.1478 | 0.3268 | 1 | 0.5874 |
| E7: FLT3_ITD | Gene: DNA methylation | 0.0601 | 1.0619 | 56 | 0.065 | 0.1486 | 0.3556 | 1 | 0.6307 |
| | interaction | | | | | | | | |
| NF1: NPM1 | Gene: Gene interaction | 0.058 | 1.0597 | 18 | 0.0634 | 0.1582 | 0.3605 | 1 | 0.6309 |
| BM Blasts | Clinical | −0.0191 | 0.9811 | 1014 | 0.0213 | 0.022 | 0.3695 | 1 | 0.6341 |
| FLT3-TKD | SNV/Indel | 0.0785 | 1.0817 | 99 | 0.0879 | 0.1298 | 0.3719 | 1 | 0.6341 |
| E7: NPM1 | Gene: DNA methylation | 0.0505 | 1.0518 | 130 | 0.0584 | 0.1476 | 0.3867 | 1 | 0.6445 |
| | interaction | | | | | | | | |
| inv(16) | Fusions | −0.2153 | 0.8063 | 24 | 0.2492 | 0.4251 | 0.3877 | 1 | 0.6445 |
| E8: NRAS | Gene: DNA methylation | −0.0633 | 0.9386 | 39 | 0.0752 | 0.1446 | 0.3999 | 1 | 0.654 |
| | interaction | | | | | | | | |
| monosomy 20 or del(20q) | CNA | −0.1026 | 0.9025 | 28 | 0.1242 | 0.1677 | 0.4088 | 1 | 0.654 |
| SHS: WT1 | Gene: DNA methylation | 0.06 | 1.0618 | 32 | 0.073 | 0.1461 | 0.4114 | 1 | 0.654 |
| | interaction | | | | | | | | |
| ETV6 | SNV/Indel | 0.0793 | 1.0825 | 25 | 0.0972 | 0.1513 | 0.4146 | 1 | 0.654 |
| E8:; FLT3_TKD | Gene: DNA methylation | −0.0568 | 0.9448 | 36 | 0.0708 | 0.1487 | 0.4223 | 1 | 0.654 |
| | interaction | | | | | | | | |
| RUNX1: SRFS2 | SNV/Indel | 0.0683 | 1.0707 | 112 | 0.0852 | 0.11 | 0.4229 | 1 | 0.654 |
| E7: DNMT3A | Gene: DNA methylation | −0.0471 | 0.954 | 119 | 0.0632 | 0.1466 | 0.4563 | 1 | 0.6862 |
| | interaction | | | | | | | | |
| t(8; 21) | Fusions | −0.208 | 0.8122 | 22 | 0.2805 | 0.4055 | 0.4585 | 1 | 0.6862 |
| E8: TET2 | Gene: DNA methylation | −0.056 | 0.9455 | 34 | 0.0757 | 0.1437 | 0.4592 | 1 | 0.6862 |
| | interaction | | | | | | | | |
| NPM1: FLT3-ITD | Gene: Gene interaction | −0.0497 | 0.9515 | 136 | 0.074 | 0.1299 | 0.5022 | 1 | 0.7421 |
| BRAF | SNV/Indel | −0.0515 | 0.9498 | 6 | 0.0807 | 0.1782 | 0.523 | 1 | 0.7644 |
| NPM1: IDH2 p140 | Gene: Gene interaction | −0.0456 | 0.9554 | 47 | 0.073 | 0.1424 | 0.5326 | 1 | 0.77 |
| E10 | DNA methylation | −0.0436 | 0.9573 | 40 | 0.0729 | 0.1673 | 0.5493 | 1 | 0.7856 |
| CBL | SNV/Indel | 0.0553 | 1.0568 | 21 | 0.0969 | 0.1589 | 0.5686 | 1 | 0.8044 |
| SHS: E7 | DNA methylation: DNA | 0.0331 | 1.0337 | 65 | 0.0658 | 0.1472 | 0.6149 | 1 | 0.8609 |
| | methylation | | | | | | | | |
| t(6; 9) | Fusions | 0.1562 | 1.1691 | 6 | 0.3251 | 0.4109 | 0.6309 | 1 | 0.8723 |
| IDH1: NPM1 | Gene: Gene interaction | 0.0335 | 1.0341 | 49 | 0.0727 | 0.1421 | 0.6448 | 1 | 0.8723 |
| JAK2 | SNV/Indel | 0.0317 | 1.0322 | 5 | 0.0697 | 0.1864 | 0.6493 | 1 | 0.8723 |
| GATA2: CEBPA-bi | Gene: Gene interaction | −0.0268 | 0.9736 | 27 | 0.0603 | 0.1576 | 0.6573 | 1 | 0.8723 |
| E4: GATA2 | Gene: DNA methylation | 0.025 | 1.0253 | 27 | 0.0572 | 0.1611 | 0.6624 | 1 | 0.8723 |
| | interaction | | | | | | | | |
| NPM1: CEBPA-mono | Gene: Gene interaction | −0.0305 | 0.97 | 27 | 0.0698 | 0.1487 | 0.6624 | 1 | 0.8723 |
| CEBPA-dm | SNV/Indel | 0.0387 | 1.0395 | 72 | 0.0915 | 0.1558 | 0.6724 | 1 | 0.8755 |
| DNMT3A; NPM1 | Gene: Gene interaction | −0.0292 | 0.9712 | 182 | 0.0703 | 0.1345 | 0.678 | 1 | 0.8755 |
| ASXL1: SRFS2 | Gene: Gene interaction | 0.0263 | 1.0266 | 25 | 0.07 | 0.1499 | 0.7077 | 1 | 0.8965 |
| IDH2 p172 | SNV/Indel | −0.0355 | 0.9652 | 33 | 0.096 | 0.152 | 0.7118 | 1 | 0.8965 |
| E11 | DNA methylation | 0.0301 | 1.0306 | 55 | 0.084 | 0.146 | 0.7197 | 1 | 0.8965 |
| E8: DNMT3A | Gene: DNA methylation | 0.0265 | 1.0269 | 45 | 0.0744 | 0.1432 | 0.7213 | 1 | 0.8965 |
| | interaction | | | | | | | | |
| HB | Clinical | −0.0285 | 0.9719 | 987 | 0.0821 | 0.1336 | 0.7282 | 1 | 0.8968 |
| RUNX1: SRFS2 | Gene: Gene interaction | −0.0235 | 0.9768 | 25 | 0.0706 | 0.1496 | 0.7397 | 1 | 0.9025 |
| RAD21 | SNV/Indel | 0.0302 | 1.0307 | 24 | 0.0972 | 0.1573 | 0.7558 | 1 | 0.9139 |
| NPM1: TET2 | Gene: Gene interaction | −0.0222 | 0.978 | 64 | 0.0751 | 0.1364 | 0.767 | 1 | 0.9179 |
| GATA2 | SNV/Indel | 0.0275 | 1.0278 | 57 | 0.0956 | 0.1401 | 0.7741 | 1 | 0.9179 |
| DNMT3A: FLT3-TKD | Gene: Gene interaction | 0.0191 | 1.0192 | 31 | 0.069 | 0.1515 | 0.7823 | 1 | 0.9179 |
| KRAS | SNV/Indel | −0.0269 | 0.9734 | 55 | 0.0995 | 0.1345 | 0.7868 | 1 | 0.9179 |
| CEBPA-sm | SNV/Indel | 0.0237 | 1.024 | 54 | 0.0927 | 0.1381 | 0.7983 | 1 | 0.9184 |
| PHF6 | SNV/Indel | 0.0243 | 1.0246 | 27 | 0.0965 | 0.1484 | 0.801 | 1 | 0.9184 |
| SHS: DNMT3A | Gene: DNA methylation | 0.0153 | 1.0154 | 88 | 0.0743 | 0.1369 | 0.8371 | 1 | 0.94 |
| | interaction | | | | | | | | |
| DNMT3A: FLT3-ITD | Gene: Gene interaction | 0.0137 | 1.0138 | 85 | 0.0709 | 0.1378 | 0.8464 | 1 | 0.94 |
| plus21 | CNA | 0.0225 | 1.0227 | 25 | 0.125 | 0.172 | 0.8572 | 1 | 0.94 |
| E4: CEBPA_bi | Gene: DNA methylation | −0.0092 | 0.9909 | 57 | 0.0549 | 0.1609 | 0.8674 | 1 | 0.94 |
| | interaction | | | | | | | | |
| DNMT3A | SNV/Indel | −0.0121 | 0.988 | 266 | 0.0784 | 0.1168 | 0.8775 | 1 | 0.94 |
| E8: PTPN11 | Gene: DNA methylation | −0.0108 | 0.9893 | 39 | 0.0708 | 0.1489 | 0.8791 | 1 | 0.94 |
| | interaction | | | | | | | | |
| IKZF1 | SNV/Indel | 0.014 | 1.0141 | 20 | 0.0941 | 0.1579 | 0.8818 | 1 | 0.94 |
| ASXL1 | SNV/Indel | 0.0124 | 1.0125 | 68 | 0.0885 | 0.129 | 0.8887 | 1 | 0.94 |
| plus13 | CNA | 0.017 | 1.0171 | 23 | 0.1255 | 0.176 | 0.8924 | 1 | 0.94 |
| KIT | SNV/Indel | 0.0122 | 1.0122 | 40 | 0.0986 | 0.1434 | 0.9018 | 1 | 0.94 |
| abnormal chr 3q | CNA | 0.0146 | 1.0147 | 12 | 0.1212 | 0.1971 | 0.9043 | 1 | 0.94 |
| DNMT3A: IDH2 p140 | Gene: Gene interaction | 0.0084 | 1.0084 | 33 | 0.0726 | 0.1453 | 0.9082 | 1 | 0.94 |
| NPM1: FLT3- | Gene: Gene interaction | 0.0063 | 1.0063 | 65 | 0.0614 | 0.1486 | 0.9178 | 1 | 0.94 |
| ITD: DNMT3A | | | | | | | | | |
| NPM1: PTPN11 | Gene: Gene interaction | −0.0063 | 0.9937 | 64 | 0.0664 | 0.1437 | 0.9243 | 1 | 0.94 |
| TET2: FLT3-ITD | Gene: Gene interaction | −0.0072 | 0.9928 | 34 | 0.0775 | 0.1422 | 0.9259 | 1 | 0.94 |
| EZH2 | SNV/Indel | 0.0032 | 1.0032 | 27 | 0.0982 | 0.1514 | 0.9743 | 1 | 0.9801 |
| ZRSR2 | SNV/Indel | −0.0024 | 0.9976 | 49 | 0.0965 | 0.1296 | 0.9801 | 1 | 0.9801 |

TABLE 7
Association of features with complete remission using multistage random effects modeling

| | | beta | hazard | | | sd | | Q-value | Q-value |
|---|---|---|---|---|---|---|---|---|---|
| Feature name | Feature class | (log-hazard) | exp(beta) | n | sd | (var) | P-value | (B-Y) | (B-H) |
| E12 | DNA methylation | −0.6996 | 0.4968 | 180 | 0.117 | 0.1642 | 0 | 0 | 0 |
| E13 | DNA methylation | −0.6658 | 0.5138 | 109 | 0.1286 | 0.1742 | 0 | 0.0001 | 0 |
| E3 | DNA methylation | 0.5042 | 1.6556 | 26 | 0.1321 | 0.2815 | 0.0001 | 0.0327 | 0.006 |
| E8:NPM1 | Gene:DNA methylation | 0.2566 | 1.2926 | 156 | 0.0761 | 0.1427 | 0.0007 | 0.1162 | 0.0212 |
| | interaction | | | | | | | | |
| E4 | DNA methylation | 0.5076 | 1.6613 | 74 | 0.1514 | 0.2202 | 0.0008 | 0.1162 | 0.0212 |
| plus11 or +11q | CNA | −0.4733 | 0.6229 | 26 | 0.1576 | 0.2566 | 0.0027 | 0.3237 | 0.0592 |
| inv(16) | Fusions | 0.3696 | 1.4472 | 24 | 0.1329 | 0.2851 | 0.0054 | 0.4942 | 0.0903 |
| WBC | Clinical | −0.1943 | 0.8234 | 1007 | 0.0709 | 0.0777 | 0.0061 | 0.4942 | 0.0903 |
| monosomy 18 or | CNA | −0.4219 | 0.6558 | 23 | 0.1552 | 0.262 | 0.0066 | 0.4942 | 0.0903 |
| del(18q) | | | | | | | | | |
| U2AF1 | SNV/Indel | −0.246 | 0.7819 | 40 | 0.0909 | 0.1427 | 0.0068 | 0.4942 | 0.0903 |
| Age of diagnosis | Demographics | −0.0724 | 0.9302 | 1021 | 0.0276 | 0.0286 | 0.0086 | 0.5388 | 0.0985 |
| NPM1 | SNV/Indel | 0.1921 | 1.2118 | 382 | 0.0735 | 0.1326 | 0.009 | 0.5388 | 0.0985 |
| FLT3-ITD | SNV/Indel | −0.2063 | 0.8136 | 223 | 0.0797 | 0.1237 | 0.0096 | 0.5388 | 0.0985 |
| E7:DNMT3A | Gene:DNA methylation | 0.1884 | 1.2073 | 119 | 0.076 | 0.1572 | 0.0132 | 0.6848 | 0.1252 |
| | interaction | | | | | | | | |
| t(9; 11) | Fusions | 0.425 | 1.5297 | 24 | 0.1804 | 0.2198 | 0.0185 | 0.8958 | 0.1637 |
| PB Blasts | Clinical | −0.0387 | 0.9621 | 1015 | 0.0174 | 0.0181 | 0.0261 | 1 | 0.2169 |
| E2 | DNA methylation | 0.3319 | 1.3936 | 29 | 0.1614 | 0.2586 | 0.0397 | 1 | 0.3109 |
| E8:FLT3_ITD | Gene:DNA methylation | −0.1754 | 0.8391 | 77 | 0.0872 | 0.146 | 0.0443 | 1 | 0.321 |
| | interaction | | | | | | | | |
| minusY | CNA | −0.3113 | 0.7325 | 35 | 0.1559 | 0.2046 | 0.0459 | 1 | 0.321 |
| SHS | DNA methylation | −0.154 | 0.8573 | 213 | 0.08 | 0.1261 | 0.0543 | 1 | 0.358 |
| HB | Clinical | 0.1951 | 1.2154 | 987 | 0.1023 | 0.1531 | 0.0565 | 1 | 0.358 |
| SHS:FLT3_ITD | Gene:DNA methylation | −0.1646 | 0.8482 | 112 | 0.0885 | 0.1461 | 0.063 | 1 | 0.372 |
| | interaction | | | | | | | | |
| BM Blasts | Clinical | 0.042 | 1.0429 | 1014 | 0.0227 | 0.0235 | 0.0643 | 1 | 0.372 |
| plus8 or +8q | CNA | −0.2262 | 0.7976 | 104 | 0.1262 | 0.1453 | 0.0731 | 1 | 0.3754 |
| PTPN11 | SNV/Indel | −0.1406 | 0.8688 | 101 | 0.0787 | 0.1328 | 0.074 | 1 | 0.3754 |
| GATA2:CEBPA-bi | Gene:Gene interaction | 0.1245 | 1.1326 | 27 | 0.0703 | 0.1698 | 0.0766 | 1 | 0.3754 |
| monosomy 7 | CNA | −0.2584 | 0.7723 | 83 | 0.1472 | 0.182 | 0.0793 | 1 | 0.3754 |
| SRFS2 | SNV/Indel | −0.1447 | 0.8653 | 74 | 0.0827 | 0.141 | 0.0801 | 1 | 0.3754 |
| E8:FLT3_TKD | Gene:DNA methylation | 0.1445 | 1.1554 | 36 | 0.083 | 0.1595 | 0.0819 | 1 | 0.3754 |
| | interaction | | | | | | | | |
| NPM1:FLT3- | Gene:Gene interaction | 0.1183 | 1.1256 | 65 | 0.0697 | 0.1636 | 0.0894 | 1 | 0.3842 |
| ITD:DNMT3A | | | | | | | | | |
| SHS:DNMT3A | Gene:DNA methylation | −0.142 | 0.8676 | 88 | 0.0836 | 0.1512 | 0.0895 | 1 | 0.3842 |
| | interaction | | | | | | | | |
| NPM1:IDH2 p140 | Gene:Gene interaction | 0.133 | 1.1422 | 47 | 0.0815 | 0.1538 | 0.1027 | 1 | 0.3993 |
| monosomy 5 or del(5q) | CNA | −0.2407 | 0.7861 | 83 | 0.148 | 0.1966 | 0.1039 | 1 | 0.3993 |
| NF1 | SNV/Indel | −0.1392 | 0.8701 | 47 | 0.0863 | 0.1429 | 0.1069 | 1 | 0.3993 |
| RAD21 | SNV/Indel | 0.1443 | 1.1552 | 24 | 0.0896 | 0.1511 | 0.1074 | 1 | 0.3993 |
| DNMT3A:FLT3-ITD | Gene:Gene interaction | 0.1214 | 1.129 | 85 | 0.0756 | 0.1565 | 0.1083 | 1 | 0.3993 |
| E8 | DNA methylation | −0.1161 | 0.8904 | 224 | 0.0732 | 0.1415 | 0.113 | 1 | 0.3993 |
| NPM1:FLT3-ITD | Gene:Gene interaction | 0.1287 | 1.1374 | 136 | 0.0815 | 0.1431 | 0.1141 | 1 | 0.3993 |
| NPM1:TET2 | Gene:Gene interaction | −0.1217 | 0.8854 | 64 | 0.0815 | 0.1497 | 0.1351 | 1 | 0.4377 |
| abnormal chr 17 | CNA | −0.2261 | 0.7976 | 63 | 0.1514 | 0.2241 | 0.1352 | 1 | 0.4377 |
| (monosomy 17 or | | | | | | | | | |
| del(17p) or | | | | | | | | | |
| abnormal 17p) | | | | | | | | | |
| E5 | DNA methylation | 0.199 | 1.2201 | 80 | 0.1335 | 0.1775 | 0.1363 | 1 | 0.4377 |
| ASXL1:RUNX1 | Gene:Gene interaction | 0.1117 | 1.1182 | 25 | 0.0753 | 0.174 | 0.1382 | 1 | 0.4377 |
| DNMT3A | SNV/Indel | −0.1137 | 0.8925 | 266 | 0.0778 | 0.124 | 0.1439 | 1 | 0.445 |
| WT1:FLT3-ITD | Gene:Gene interaction | −0.1203 | 0.8866 | 31 | 0.0831 | 0.1645 | 0.1475 | 1 | 0.4458 |
| E8:TET2 | Gene:DNA methylation | 0.1235 | 1.1314 | 34 | 0.0871 | 0.1587 | 0.156 | 1 | 0.4612 |
| | interaction | | | | | | | | |
| NPM1:NRAS | Gene:Gene interaction | 0.1136 | 1.1203 | 67 | 0.0832 | 0.1456 | 0.1719 | 1 | 0.497 |
| NRAS:TET2 | Gene:Gene interaction | 0.1155 | 1.1225 | 25 | 0.086 | 0.1641 | 0.1791 | 1 | 0.5003 |
| KIT | SNV/Indel | −0.1227 | 0.8846 | 40 | 0.0916 | 0.137 | 0.1805 | 1 | 0.5003 |
| BCOR | SNV/Indel | −0.1203 | 0.8867 | 63 | 0.092 | 0.133 | 0.1911 | 1 | 0.5091 |
| FLT3-TKD | SNV/Indel | 0.1038 | 1.1094 | 99 | 0.0798 | 0.1342 | 0.1934 | 1 | 0.5091 |
| NF1:NPM1 | Gene:Gene interaction | 0.101 | 1.1063 | 18 | 0.078 | 0.1706 | 0.1952 | 1 | 0.5091 |
| SRFS2:IDH2_p140 | Gene:Gene interaction | −0.1005 | 0.9044 | 29 | 0.0783 | 0.1676 | 0.1993 | 1 | 0.5098 |
| ZRSR2 | SNV/Indel | −0.1121 | 0.8939 | 49 | 0.092 | 0.1342 | 0.2229 | 1 | 0.5593 |
| PHF6 | SNV/Indel | 0.0966 | 1.1014 | 27 | 0.0828 | 0.1566 | 0.2434 | 1 | 0.5995 |
| ETV6 | SNV/Indel | −0.097 | 0.9076 | 25 | 0.0867 | 0.1523 | 0.2633 | 1 | 0.6352 |
| DNMT3A:NRAS | Gene:Gene interaction | −0.0989 | 0.9058 | 47 | 0.0892 | 0.1504 | 0.2675 | 1 | 0.6352 |
| IDH2 p172 | SNV/Indel | −0.0901 | 0.9139 | 33 | 0.0837 | 0.154 | 0.2817 | 1 | 0.6572 |
| DNMT3A:PTPN11 | Gene:Gene interaction | 0.0912 | 1.0955 | 34 | 0.0858 | 0.1588 | 0.2879 | 1 | 0.6601 |
| SHS:E8 | DNA methylation:DNA | −0.0845 | 0.919 | 71 | 0.0831 | 0.1522 | 0.3096 | 1 | 0.6797 |
| | methylation | | | | | | | | |
| ASXL1:SRFS2 | Gene:Gene interaction | −0.0758 | 0.927 | 25 | 0.0748 | 0.1737 | 0.311 | 1 | 0.6797 |
| del(7q) | CNA | −0.1536 | 0.8576 | 53 | 0.1519 | 0.1975 | 0.3117 | 1 | 0.6797 |
| NPM1:CEBPA-mono | Gene:Gene interaction | 0.0817 | 1.0851 | 27 | 0.0821 | 0.162 | 0.3199 | 1 | 0.6848 |
| ASXL1 | SNV/Indel | −0.0846 | 0.9189 | 68 | 0.0858 | 0.1399 | 0.3244 | 1 | 0.6848 |
| E8:NRAS | Gene:DNA methylation | 0.0824 | 1.0859 | 39 | 0.0878 | 0.1534 | 0.3479 | 1 | 0.723 |
| | interaction | | | | | | | | |
| E8:DNMT3A | Gene:DNA methylation | 0.0751 | 1.078 | 45 | 0.0846 | 0.1545 | 0.3747 | 1 | 0.7553 |
| | interaction | | | | | | | | |
| plus13 | CNA | −0.1418 | 0.8678 | 23 | 0.162 | 0.2458 | 0.3813 | 1 | 0.7553 |
| E7 | DNA methylation | 0.0578 | 1.0595 | 144 | 0.0666 | 0.1532 | 0.3853 | 1 | 0.7553 |
| CBL | SNV/Indel | 0.0709 | 1.0735 | 21 | 0.0818 | 0.1609 | 0.3862 | 1 | 0.7553 |
| DNMT3A:NPM1 | Gene:Gene interaction | 0.0657 | 1.0679 | 182 | 0.0767 | 0.1451 | 0.3921 | 1 | 0.7553 |
| DNMT3A:FLT3-TKD | Gene:Gene interaction | −0.0707 | 0.9317 | 31 | 0.084 | 0.1591 | 0.3999 | 1 | 0.7553 |
| E11 | DNA methylation | 0.0666 | 1.0688 | 55 | 0.0796 | 0.1459 | 0.4032 | 1 | 0.7553 |
| DNMT3A:IDH2 p140 | Gene:Gene interaction | −0.0684 | 0.9339 | 33 | 0.0838 | 0.1642 | 0.4145 | 1 | 0.7656 |
| abnormal chr 12 | CNA | −0.1253 | 0.8823 | 40 | 0.1576 | 0.2192 | 0.4268 | 1 | 0.7764 |
| (monosomy 12 or | | | | | | | | | |
| del(12p) or | | | | | | | | | |
| abnormal 12p) | | | | | | | | | |
| TET2:FLT3-ITD | Gene:Gene interaction | 0.0694 | 1.0719 | 34 | 0.0892 | 0.1597 | 0.4361 | 1 | 0.7764 |
| SF3B1 | SNV/Indel | −0.0699 | 0.9325 | 37 | 0.0914 | 0.1438 | 0.4442 | 1 | 0.7764 |
| MLL | SNV/Indel | −0.0608 | 0.941 | 14 | 0.0801 | 0.1614 | 0.4481 | 1 | 0.7764 |
| IDH1 | SNV/Indel | −0.0588 | 0.9429 | 88 | 0.0785 | 0.1358 | 0.4538 | 1 | 0.7764 |
| STAG2 | SNV/Indel | −0.064 | 0.938 | 30 | 0.0867 | 0.1509 | 0.4605 | 1 | 0.7764 |
| NPM1:PTPN11 | Gene:Gene interaction | 0.0552 | 1.0567 | 64 | 0.0749 | 0.1537 | 0.4612 | 1 | 0.7764 |
| E6 | DNA methylation | −0.1217 | 0.8854 | 38 | 0.1674 | 0.2199 | 0.4672 | 1 | 0.7767 |
| PTEN | SNV/Indel | −0.0361 | 0.9645 | 5 | 0.0526 | 0.1783 | 0.4926 | 1 | 0.8088 |
| t(6; 9) | Fusions | −0.1272 | 0.8806 | 6 | 0.1921 | 0.328 | 0.5078 | 1 | 0.8169 |
| SHS:WT1 | Gene:DNA methylation | −0.0535 | 0.9479 | 32 | 0.0817 | 0.1664 | 0.5123 | 1 | 0.8169 |
| | interaction | | | | | | | | |
| abnormal chr 3q | CNA | 0.0915 | 1.0958 | 12 | 0.1423 | 0.2808 | 0.5201 | 1 | 0.8169 |
| TET2 | SNV/Indel | −0.052 | 0.9493 | 137 | 0.0812 | 0.1244 | 0.5221 | 1 | 0.8169 |
| NPM1:WT1 | Gene:Gene interaction | −0.0503 | 0.9509 | 36 | 0.0807 | 0.1622 | 0.5331 | 1 | 0.8245 |
| plus22 | CNA | −0.0971 | 0.9075 | 25 | 0.1591 | 0.2387 | 0.5416 | 1 | 0.828 |
| monosomy 20 or | CNA | −0.096 | 0.9084 | 28 | 0.1623 | 0.2327 | 0.5541 | 1 | 0.8318 |
| del(20q) | | | | | | | | | |
| t(v; 11) | Fusions | −0.0974 | 0.9072 | 39 | 0.1656 | 0.1974 | 0.5566 | 1 | 0.8318 |
| DNMT3A:TET2 | Gene:Gene interaction | −0.0497 | 0.9515 | 31 | 0.0875 | 0.1587 | 0.5697 | 1 | 0.8418 |
| Gender | Demographics | −0.0402 | 0.9606 | 1021 | 0.0757 | 0.0798 | 0.5956 | 1 | 0.8705 |
| Platelets | Clinical | 0.0274 | 1.0278 | 1002 | 0.0557 | 0.0596 | 0.6221 | 1 | 0.8993 |
| BRAF | SNV/Indel | −0.0295 | 0.971 | 6 | 0.0647 | 0.1742 | 0.649 | 1 | 0.9174 |
| SHS:NPM1 | Gene:DNA methylation | −0.0365 | 0.9641 | 117 | 0.081 | 0.1482 | 0.6521 | 1 | 0.9174 |
| | interaction | | | | | | | | |
| complex karyotype | CNA | −0.0599 | 0.9419 | 121 | 0.1358 | 0.1831 | 0.6591 | 1 | 0.9174 |
| LDH | Clinical | −0.0303 | 0.9701 | 759 | 0.0694 | 0.193 | 0.6622 | 1 | 0.9174 |
| E4:GATA2 | Gene:DNA methylation | 0.0279 | 1.0282 | 27 | 0.07 | 0.1695 | 0.6906 | 1 | 0.9373 |
| | interaction | | | | | | | | |
| JAK2 | SNV/Indel | 0.0237 | 1.024 | 5 | 0.0605 | 0.1754 | 0.6952 | 1 | 0.9373 |
| NPM1:FLT3-TKD | Gene:Gene interaction | 0.0281 | 1.0285 | 60 | 0.0739 | 0.1538 | 0.7038 | 1 | 0.9373 |
| CEBPA-dm | SNV/Indel | 0.0304 | 1.0309 | 72 | 0.0815 | 0.1508 | 0.709 | 1 | 0.9373 |
| E11:NPM1 | Gene:DNA methylation | 0.0236 | 1.0239 | 36 | 0.0654 | 0.1663 | 0.7181 | 1 | 0.9373 |
| | interaction | | | | | | | | |
| RUNX1 | SNV/Indel | −0.0302 | 0.9703 | 112 | 0.0882 | 0.1233 | 0.732 | 1 | 0.9373 |
| DNMT3A:IDH1 | Gene:Gene interaction | −0.0294 | 0.9711 | 42 | 0.0859 | 0.1577 | 0.7324 | 1 | 0.9373 |
| IDH1:NPM1 | Gene:Gene interaction | 0.0267 | 1.027 | 49 | 0.0811 | 0.1545 | 0.742 | 1 | 0.9373 |
| IKZF1 | SNV/Indel | −0.0271 | 0.9733 | 20 | 0.0823 | 0.1597 | 0.7422 | 1 | 0.9373 |
| E7:NPM1 | Gene:DNA methylation | 0.0215 | 1.0217 | 130 | 0.0668 | 0.1598 | 0.7477 | 1 | 0.9373 |
| | interaction | | | | | | | | |
| SF3A1 | SNV/Indel | −0.0203 | 0.9799 | 9 | 0.0679 | 0.1718 | 0.765 | 1 | 0.9373 |
| E9 | DNA methylation | 0.0229 | 1.0231 | 22 | 0.0767 | 0.1623 | 0.7657 | 1 | 0.9373 |
| del(9q) | CNA | −0.0461 | 0.955 | 15 | 0.1644 | 0.2323 | 0.7793 | 1 | 0.9373 |
| abnormal chr 7 (other) | CNA | 0.0418 | 1.0427 | 12 | 0.1577 | 0.2652 | 0.7909 | 1 | 0.9373 |
| plus21 | CNA | 0.0418 | 1.0427 | 25 | 0.1612 | 0.2279 | 0.7954 | 1 | 0.9373 |
| E7:FLT3_ITD | Gene:DNA methylation | 0.0193 | 1.0195 | 56 | 0.0772 | 0.1626 | 0.8028 | 1 | 0.9373 |
| | interaction | | | | | | | | |
| SF1 | SNV/Indel | −0.0165 | 0.9836 | 9 | 0.0675 | 0.1722 | 0.8064 | 1 | 0.9373 |
| NRAS | SNV/Indel | 0.0193 | 1.0195 | 167 | 0.0806 | 0.112 | 0.8111 | 1 | 0.9373 |
| GATA2 | SNV/Indel | −0.0199 | 0.9803 | 57 | 0.0845 | 0.1382 | 0.814 | 1 | 0.9373 |
| E10 | DNA methylation | −0.0146 | 0.9855 | 40 | 0.0657 | 0.1597 | 0.8239 | 1 | 0.9373 |
| CEBPA-sm | SNV/Indel | −0.0183 | 0.9819 | 54 | 0.0843 | 0.1373 | 0.828 | 1 | 0.9373 |
| WT1 | SNV/Indel | −0.0174 | 0.9827 | 86 | 0.082 | 0.1363 | 0.8316 | 1 | 0.9373 |
| abnormal chr 4 | CNA | 0.0283 | 1.0287 | 17 | 0.1475 | 0.2737 | 0.8477 | 1 | 0.9474 |
| (monosomy 4 or | | | | | | | | | |
| del(4p) or | | | | | | | | | |
| abnormal 4p) | | | | | | | | | |
| RUNX1:SRFS2 | Gene:Gene interaction | 0.0125 | 1.0126 | 25 | 0.0759 | 0.1738 | 0.8694 | 1 | 0.9611 |
| MPL | SNV/Indel | −0.0119 | 0.9882 | 11 | 0.0754 | 0.1659 | 0.8744 | 1 | 0.9611 |
| ECOG Performance | Clinical | −0.0067 | 0.9933 | 880 | 0.0541 | 0.0575 | 0.9011 | 1 | 0.9742 |
| status | | | | | | | | | |
| E8:PTPN11 | Gene:DNA methylation | −0.0094 | 0.9906 | 39 | 0.083 | 0.1579 | 0.9096 | 1 | 0.9742 |
| | interaction | | | | | | | | |
| SHS:E7 | DNA methylation:DNA | 0.0083 | 1.0083 | 65 | 0.0776 | 0.1612 | 0.915 | 1 | 0.9742 |
| | methylation | | | | | | | | |
| E4:CEBPA_bi | Gene:DNA methylation | −0.0071 | 0.9929 | 57 | 0.0679 | 0.1687 | 0.9169 | 1 | 0.9742 |
| | interaction | | | | | | | | |
| IDH2 p140 | SNV/Indel | 0.0073 | 1.0073 | 91 | 0.0753 | 0.1367 | 0.9229 | 1 | 0.9742 |
| t(8; 21) | Fusions | −0.0127 | 0.9873 | 22 | 0.1661 | 0.2671 | 0.9388 | 1 | 0.9832 |
| E8:WT1 | Gene:DNA methylation | −0.0049 | 0.9951 | 40 | 0.0801 | 0.1623 | 0.9508 | 1 | 0.988 |
| | interaction | | | | | | | | |
| Splenomegaly | Clinical | −0.0008 | 0.9992 | 69 | 0.0282 | 0.206 | 0.9781 | 1 | 0.9922 |
| tAML | Clinical | −0.0008 | 0.9992 | 7 | 0.0282 | 0.206 | 0.9781 | 1 | 0.9922 |
| KRAS | SNV/Indel | −0.0022 | 0.9978 | 55 | 0.0934 | 0.1281 | 0.9811 | 1 | 0.9922 |
| EZH2 | SNV/Indel | −0.0017 | 0.9983 | 27 | 0.0895 | 0.1484 | 0.9847 | 1 | 0.9922 |
| TP53 | SNV/Indel | −0.0005 | 0.9995 | 88 | 0.091 | 0.1443 | 0.9952 | 1 | 0.9952 |

TABLE 8
Association of features with non-remission death using multistage random effects modeling

| | | beta | hazard | | | sd | | Q-value | Q-value |
|---|---|---|---|---|---|---|---|---|---|
| Feature name | Feature class | (log-hazard) | exp(beta) | n | sd | (var) | P-value | (B-Y) | (B-H) |
| Age of diagnosis | Demographics | 0.2226 | 1.2493 | 1021 | 0.0419 | 0.0451 | 0 | 0.0001 | 0 |
| abnormal chr 17 | CNA | 0.5447 | 1.7241 | 63 | 0.1346 | 0.2044 | 0.0001 | 0.019 | 0.0035 |
| (monosomy 17 or | | | | | | | | | |
| del(17p) or | | | | | | | | | |
| abnormal 17p) | | | | | | | | | |
| monosomy 7 | CNA | 0.4296 | 1.5366 | 83 | 0.128 | 0.1625 | 0.0008 | 0.1675 | 0.0306 |
| WBC | Clinical | 0.2459 | 1.2788 | 1007 | 0.0742 | 0.0874 | 0.0009 | 0.1675 | 0.0306 |
| SHS | DNA methylation | 0.2973 | 1.3462 | 213 | 0.1054 | 0.1578 | 0.0048 | 0.6954 | 0.1271 |
| NRAS | SNV/Indel | 0.2761 | 1.318 | 167 | 0.1062 | 0.1519 | 0.0093 | 1 | 0.2062 |
| SF3A1 | SNV/Indel | 0.1702 | 1.1855 | 9 | 0.0669 | 0.215 | 0.011 | 1 | 0.2083 |
| SHS:FLT3_ITD | Gene:DNA methylation interaction | 0.2181 | 1.2437 | 112 | 0.0892 | 0.1804 | 0.0144 | 1 | 0.2213 |
| E12 | DNA methylation | 0.2757 | 1.3174 | 180 | 0.1133 | 0.1764 | 0.015 | 1 | 0.2213 |
| monosomy 5 or | CNA | 0.314 | 1.3688 | 83 | 0.1342 | 0.1879 | 0.0193 | 1 | 0.257 |
| del(5q) | | | | | | | | | |
| E6 | DNA methylation | 0.5058 | 1.6583 | 38 | 0.2234 | 0.3124 | 0.0236 | 1 | 0.2614 |
| SRFS2:IDH2_p140 | Gene:Gene interaction | −0.2027 | 0.8165 | 29 | 0.0896 | 0.1964 | 0.0236 | 1 | 0.2614 |
| TP53 | SNV/Indel | 0.2484 | 1.282 | 88 | 0.1117 | 0.167 | 0.0262 | 1 | 0.268 |
| E5 | DNA methylation | 0.4164 | 1.5164 | 80 | 0.1942 | 0.2558 | 0.032 | 1 | 0.3042 |
| del(7q) | CNA | 0.2833 | 1.3275 | 53 | 0.1381 | 0.187 | 0.0402 | 1 | 0.3566 |
| E9 | DNA methylation | −0.1663 | 0.8468 | 22 | 0.0857 | 0.2028 | 0.0522 | 1 | 0.4115 |
| Gender | Demographics | 0.2037 | 1.2259 | 1021 | 0.1066 | 0.1199 | 0.056 | 1 | 0.4115 |
| ECOG Performance | Clinical | 0.1345 | 1.144 | 880 | 0.0707 | 0.0785 | 0.057 | 1 | 0.4115 |
| status | | | | | | | | | |
| E11:NPM1 | Gene:DNA methylation interaction | −0.1442 | 0.8658 | 36 | 0.0771 | 0.2085 | 0.0617 | 1 | 0.4115 |
| NPM1:CEBPA- | Gene:Gene interaction | −0.1527 | 0.8584 | 27 | 0.0821 | 0.2098 | 0.0628 | 1 | 0.4115 |
| mono | | | | | | | | | |
| plus22 | CNA | −0.2554 | 0.7746 | 25 | 0.1384 | 0.216 | 0.065 | 1 | 0.4115 |
| MPL | SNV/Indel | 0.1252 | 1.1334 | 11 | 0.0691 | 0.2154 | 0.0699 | 1 | 0.4223 |
| SHS:DNMT3A | Gene:DNA methylation interaction | 0.1563 | 1.1692 | 88 | 0.0883 | 0.188 | 0.0769 | 1 | 0.4309 |
| complex karyotype | CNA | 0.22 | 1.2461 | 121 | 0.1247 | 0.1903 | 0.0778 | 1 | 0.4309 |
| DNMT3A:FLT3- | Gene:Gene interaction | 0.1254 | 1.1336 | 31 | 0.0774 | 0.2136 | 0.1052 | 1 | 0.5594 |
| TKD | | | | | | | | | |
| E8:DNMT3A | Gene:DNA methylation interaction | 0.1204 | 1.128 | 45 | 0.0802 | 0.2092 | 0.1334 | 1 | 0.6802 |
| HB | Clinical | −0.1396 | 0.8697 | 987 | 0.0997 | 0.2002 | 0.1614 | 1 | 0.6802 |
| E7 | DNA methylation | 0.1201 | 1.1276 | 144 | 0.0858 | 0.2009 | 0.1615 | 1 | 0.6802 |
| NF1:NPM1 | Gene:Gene interaction | 0.0871 | 1.091 | 18 | 0.0623 | 0.2208 | 0.1625 | 1 | 0.6802 |
| E7:NPM1 | Gene:DNA methylation interaction | 0.0898 | 1.0939 | 130 | 0.0647 | 0.2033 | 0.1653 | 1 | 0.6802 |
| ZRSR2 | SNV/Indel | −0.1516 | 0.8594 | 49 | 0.1119 | 0.1667 | 0.1754 | 1 | 0.6802 |
| SHS:E8 | DNA methylation:DNA | 0.121 | 1.1286 | 71 | 0.09 | 0.1902 | 0.1788 | 1 | 0.6802 |
| | methylation | | | | | | | | |
| SF1 | SNV/Indel | −0.1274 | 0.8804 | 9 | 0.096 | 0.1956 | 0.1845 | 1 | 0.6802 |
| NPM1:WT1 | Gene:Gene interaction | 0.1076 | 1.1136 | 36 | 0.0823 | 0.2002 | 0.1909 | 1 | 0.6802 |
| SF3B1 | SNV/Indel | 0.1369 | 1.1467 | 37 | 0.1057 | 0.1816 | 0.1954 | 1 | 0.6802 |
| NF1 | SNV/Indel | 0.1361 | 1.1458 | 47 | 0.1077 | 0.1704 | 0.2064 | 1 | 0.6802 |
| E2 | DNA methylation | −0.2475 | 0.7808 | 29 | 0.1965 | 0.393 | 0.208 | 1 | 0.6802 |
| E11 | DNA methylation | 0.1183 | 1.1256 | 55 | 0.0949 | 0.1883 | 0.2123 | 1 | 0.6802 |
| LDH | Clinical | 0.0786 | 1.0818 | 759 | 0.064 | 0.2235 | 0.2197 | 1 | 0.6802 |
| plus8 or +8q | CNA | 0.156 | 1.1688 | 104 | 0.1274 | 0.1564 | 0.2208 | 1 | 0.6802 |
| monosomy 20 or | CNA | −0.1828 | 0.833 | 28 | 0.1496 | 0.2114 | 0.2219 | 1 | 0.6802 |
| del(20q) | | | | | | | | | |
| E4 | DNA methylation | −0.2815 | 0.7547 | 74 | 0.238 | 0.3585 | 0.2369 | 1 | 0.6802 |
| BCOR | SNV/Indel | 0.1302 | 1.1391 | 63 | 0.1102 | 0.1544 | 0.2375 | 1 | 0.6802 |
| SHS:NPM1 | Gene:DNA methylation interaction | 0.1006 | 1.1058 | 117 | 0.0856 | 0.1865 | 0.2397 | 1 | 0.6802 |
| E8 | DNA methylation | 0.1108 | 1.1172 | 224 | 0.0947 | 0.1817 | 0.2419 | 1 | 0.6802 |
| t(8; 21) | Fusions | −0.2282 | 0.7959 | 22 | 0.1952 | 0.3949 | 0.2424 | 1 | 0.6802 |
| STAG2 | SNV/Indel | 0.123 | 1.1309 | 30 | 0.1062 | 0.1812 | 0.2466 | 1 | 0.6802 |
| PTEN | SNV/Indel | 0.0836 | 1.0872 | 5 | 0.0728 | 0.2122 | 0.2505 | 1 | 0.6802 |
| Platelets | Clinical | −0.0897 | 0.9142 | 1002 | 0.0781 | 0.0873 | 0.2506 | 1 | 0.6802 |
| PTPN11 | SNV/Indel | 0.123 | 1.1309 | 101 | 0.1084 | 0.1627 | 0.2565 | 1 | 0.6823 |
| Splenomegaly | Clinical | 0.0357 | 1.0364 | 69 | 0.033 | 0.2333 | 0.2787 | 1 | 0.713 |
| tAML | Clinical | 0.0357 | 1.0364 | 7 | 0.033 | 0.2333 | 0.2787 | 1 | 0.713 |
| abnormal chr 7 | CNA | 0.1381 | 1.148 | 12 | 0.1292 | 0.2746 | 0.2852 | 1 | 0.7156 |
| (other) | | | | | | | | | |
| DNMT3A:IDH1 | Gene:Gene interaction | 0.0985 | 1.1036 | 42 | 0.0938 | 0.1968 | 0.2937 | 1 | 0.7234 |
| E7:DNMT3A | Gene:DNA methylation interaction | −0.0687 | 0.9336 | 119 | 0.0662 | 0.2031 | 0.2995 | 1 | 0.7243 |
| RUNX1:SRFS2 | Gene:Gene interaction | −0.0943 | 0.91 | 25 | 0.0939 | 0.1966 | 0.3151 | 1 | 0.7326 |
| IDH2 p140 | SNV/Indel | −0.0924 | 0.9118 | 91 | 0.0927 | 0.1737 | 0.3191 | 1 | 0.7326 |
| GATA2 | SNV/Indel | 0.1054 | 1.1112 | 57 | 0.1059 | 0.183 | 0.3195 | 1 | 0.7326 |
| NPM1 | SNV/Indel | −0.0978 | 0.9069 | 382 | 0.0999 | 0.182 | 0.3278 | 1 | 0.7389 |
| inv(16) | Fusions | 0.1844 | 1.2025 | 24 | 0.2113 | 0.4271 | 0.3826 | 1 | 0.8306 |
| E3 | DNA methylation | 0.1823 | 1.2 | 26 | 0.2116 | 0.4267 | 0.3888 | 1 | 0.8306 |
| t(9; 11) | Fusions | 0.201 | 1.2227 | 24 | 0.2339 | 0.4274 | 0.3901 | 1 | 0.8306 |
| DNMT3A:IDH2 | Gene:Gene interaction | −0.0778 | 0.9251 | 33 | 0.0912 | 0.1944 | 0.3934 | 1 | 0.8306 |
| p140 | | | | | | | | | |
| WT1 | SNV/Indel | 0.0833 | 1.0869 | 86 | 0.1033 | 0.1744 | 0.42 | 1 | 0.8571 |
| DNMT3A:PTPN11 | Gene:Gene interaction | −0.0617 | 0.9401 | 34 | 0.0791 | 0.2069 | 0.4352 | 1 | 0.8571 |
| plus13 | CNA | 0.1135 | 1.1202 | 23 | 0.1476 | 0.2232 | 0.442 | 1 | 0.8571 |
| NRAS:TET2 | Gene:Gene interaction | 0.055 | 1.0565 | 25 | 0.073 | 0.2156 | 0.4516 | 1 | 0.8571 |
| abnormal chr 4 | CNA | 0.1006 | 1.1059 | 17 | 0.136 | 0.2435 | 0.4594 | 1 | 0.8571 |
| (monosomy 4 or | | | | | | | | | |
| del(4p) or abnormal | | | | | | | | | |
| 4p) | | | | | | | | | |
| E8:NPM1 | Gene:DNA methylation interaction | −0.065 | 0.9371 | 156 | 0.088 | 0.189 | 0.4602 | 1 | 0.8571 |
| E10 | DNA methylation | −0.0601 | 0.9417 | 40 | 0.0818 | 0.2028 | 0.4625 | 1 | 0.8571 |
| plus21 | CNA | −0.1045 | 0.9008 | 25 | 0.1444 | 0.2217 | 0.4693 | 1 | 0.8571 |
| plus11 or +11q | CNA | 0.1017 | 1.1071 | 26 | 0.1406 | 0.2027 | 0.4694 | 1 | 0.8571 |
| DNMT3A:NRAS | Gene:Gene interaction | 0.0686 | 1.071 | 47 | 0.0951 | 0.1965 | 0.4705 | 1 | 0.8571 |
| FLT3-ITD | SNV/Indel | 0.0759 | 1.0789 | 223 | 0.1068 | 0.1539 | 0.4769 | 1 | 0.8571 |
| E7:FLT3_ITD | Gene:DNA methylation interaction | −0.0474 | 0.9537 | 56 | 0.0679 | 0.2058 | 0.4848 | 1 | 0.8597 |
| FLT3-TKD | SNV/Indel | 0.0723 | 1.0749 | 99 | 0.107 | 0.1765 | 0.4996 | 1 | 0.8742 |
| E4:CEBPA_bi | Gene:DNA methylation interaction | −0.0427 | 0.9582 | 57 | 0.0643 | 0.2185 | 0.507 | 1 | 0.8757 |
| CEBPA-sm | SNV/Indel | 0.066 | 1.0683 | 54 | 0.1054 | 0.182 | 0.5308 | 1 | 0.9051 |
| ASXL1 | SNV/Indel | −0.0633 | 0.9387 | 68 | 0.1042 | 0.1579 | 0.5434 | 1 | 0.9054 |
| RAD21 | SNV/Indel | 0.0458 | 1.0469 | 24 | 0.0758 | 0.2138 | 0.5454 | 1 | 0.9054 |
| SHS:WT1 | Gene:DNA methylation interaction | −0.0528 | 0.9486 | 32 | 0.0887 | 0.1915 | 0.5519 | 1 | 0.9054 |
| abnormal chr 3q | CNA | −0.0735 | 0.9291 | 12 | 0.1283 | 0.2589 | 0.5665 | 1 | 0.9054 |
| ASXL1:RUNX1 | Gene:Gene interaction | −0.0545 | 0.947 | 25 | 0.0961 | 0.1973 | 0.5708 | 1 | 0.9054 |
| JAK2 | SNV/Indel | 0.0313 | 1.0318 | 5 | 0.0561 | 0.2201 | 0.577 | 1 | 0.9054 |
| E13 | DNA methylation | 0.0661 | 1.0683 | 109 | 0.1205 | 0.1838 | 0.5835 | 1 | 0.9054 |
| SRFS2 | SNV/Indel | 0.0524 | 1.0538 | 74 | 0.0982 | 0.1691 | 0.5935 | 1 | 0.9054 |
| E8:FLT3_ITD | Gene:DNA methylation interaction | 0.0484 | 1.0496 | 77 | 0.0913 | 0.187 | 0.5957 | 1 | 0.9054 |
| NPM1:IDH2 p140 | Gene:Gene interaction | −0.0448 | 0.9562 | 47 | 0.0851 | 0.2053 | 0.5991 | 1 | 0.9054 |
| E8:TET2 | Gene:DNA methylation interaction | −0.0423 | 0.9586 | 34 | 0.0844 | 0.2046 | 0.6159 | 1 | 0.9204 |
| abnormal chr 12 | CNA | 0.0676 | 1.0699 | 40 | 0.1413 | 0.2026 | 0.6324 | 1 | 0.9213 |
| (monosomy 12 or | | | | | | | | | |
| del(12p) or | | | | | | | | | |
| abnormal 12p) | | | | | | | | | |
| NPM1:NRAS | Gene:Gene interaction | −0.0389 | 0.9618 | 67 | 0.0823 | 0.2086 | 0.6367 | 1 | 0.9213 |
| BM Blasts | Clinical | 0.0147 | 1.0148 | 1014 | 0.0325 | 0.0345 | 0.6515 | 1 | 0.9213 |
| t(v; 11) | Fusions | 0.099 | 1.1041 | 39 | 0.2201 | 0.2821 | 0.6527 | 1 | 0.9213 |
| CEBPA-dm | SNV/Indel | 0.0392 | 1.0399 | 72 | 0.0878 | 0.2028 | 0.6556 | 1 | 0.9213 |
| BRAF | SNV/Indel | −0.0348 | 0.9658 | 6 | 0.079 | 0.2112 | 0.6592 | 1 | 0.9213 |
| PHF6 | SNV/Indel | 0.0427 | 1.0436 | 27 | 0.0987 | 0.1892 | 0.665 | 1 | 0.9213 |
| E8:FLT3_TKD | Gene:DNA methylation interaction | −0.0289 | 0.9715 | 36 | 0.0713 | 0.2142 | 0.6854 | 1 | 0.9398 |
| NPM1:TET2 | Gene:Gene interaction | −0.0352 | 0.9654 | 64 | 0.0904 | 0.1937 | 0.6965 | 1 | 0.9406 |
| E8:PTPN11 | Gene:DNA methylation interaction | −0.0292 | 0.9712 | 39 | 0.0782 | 0.2135 | 0.7085 | 1 | 0.9406 |
| DNMT3A | SNV/Indel | −0.0372 | 0.9635 | 266 | 0.1007 | 0.1588 | 0.7116 | 1 | 0.9406 |
| ETV6 | SNV/Indel | −0.0387 | 0.962 | 25 | 0.1058 | 0.1821 | 0.7143 | 1 | 0.9406 |
| IDH2 p172 | SNV/Indel | −0.0355 | 0.9651 | 33 | 0.1028 | 0.1845 | 0.7297 | 1 | 0.9451 |
| TET2:FLT3-ITD | Gene:Gene interaction | −0.0312 | 0.9693 | 34 | 0.0911 | 0.199 | 0.7319 | 1 | 0.9451 |
| RUNX1 | SNV/Indel | 0.0346 | 1.0352 | 112 | 0.1046 | 0.1425 | 0.7406 | 1 | 0.9472 |
| t(6; 9) | Fusions | 0.0775 | 1.0806 | 6 | 0.2556 | 0.3739 | 0.7618 | 1 | 0.9504 |
| ASXL1:SRFS2 | Gene:Gene interaction | 0.0281 | 1.0285 | 25 | 0.0929 | 0.1964 | 0.7621 | 1 | 0.9504 |
| del(9q) | CNA | 0.0374 | 1.0382 | 15 | 0.1287 | 0.271 | 0.7711 | 1 | 0.9504 |
| WT1:FLT3-ITD | Gene:Gene interaction | −0.0249 | 0.9754 | 31 | 0.086 | 0.195 | 0.7718 | 1 | 0.9504 |
| KRAS | SNV/Indel | −0.0259 | 0.9745 | 55 | 0.1072 | 0.1825 | 0.8094 | 1 | 0.9743 |
| PB Blasts | Clinical | 0.0058 | 1.0058 | 1015 | 0.0257 | 0.0272 | 0.8215 | 1 | 0.9743 |
| IDH1 | SNV/Indel | 0.0229 | 1.0231 | 88 | 0.1021 | 0.1676 | 0.8227 | 1 | 0.9743 |
| IKZF1 | SNV/Indel | −0.0215 | 0.9788 | 20 | 0.0986 | 0.1901 | 0.8276 | 1 | 0.9743 |
| NPM1:FLT3- | Gene:Gene interaction | −0.014 | 0.9861 | 65 | 0.069 | 0.204 | 0.8394 | 1 | 0.9743 |
| ITD:DNMT3A | | | | | | | | | |
| monosomy 18 or | CNA | −0.0277 | 0.9727 | 23 | 0.1434 | 0.2096 | 0.8471 | 1 | 0.9743 |
| del(18q) | | | | | | | | | |
| NPM1:FLT3-ITD | Gene:Gene interaction | 0.0159 | 1.016 | 136 | 0.0845 | 0.1852 | 0.8508 | 1 | 0.9743 |
| U2AF1 | SNV/Indel | 0.019 | 1.0191 | 40 | 0.1098 | 0.167 | 0.8629 | 1 | 0.9743 |
| E8:NRAS | Gene:DNA methylation interaction | −0.0136 | 0.9865 | 39 | 0.0789 | 0.2136 | 0.8635 | 1 | 0.9743 |
| SHS:E7 | DNA methylation:DNA methylation | −0.0108 | 0.9893 | 65 | 0.0686 | 0.205 | 0.8751 | 1 | 0.9743 |
| MLL | SNV/Indel | −0.0124 | 0.9877 | 14 | 0.0861 | 0.1997 | 0.8858 | 1 | 0.9743 |
| GATA2:CEBPA-bi | Gene:Gene interaction | −0.0064 | 0.9937 | 27 | 0.0452 | 0.2263 | 0.8883 | 1 | 0.9743 |
| IDH1:NPM1 | Gene:Gene interaction | −0.012 | 0.9881 | 49 | 0.0877 | 0.2043 | 0.8915 | 1 | 0.9743 |
| DNMT3A:NPM1 | Gene:Gene interaction | 0.0108 | 1.0109 | 182 | 0.0834 | 0.1884 | 0.8966 | 1 | 0.9743 |
| CBL | SNV/Indel | 0.0105 | 1.0106 | 21 | 0.0956 | 0.197 | 0.9126 | 1 | 0.9743 |
| DNMT3A:FLT3- | Gene:Gene interaction | −0.0098 | 0.9902 | 85 | 0.0906 | 0.1858 | 0.9137 | 1 | 0.9743 |
| ITD | | | | | | | | | |
| NPM1:FLT3-TKD | Gene:Gene interaction | −0.0081 | 0.9919 | 60 | 0.0835 | 0.2046 | 0.9228 | 1 | 0.9743 |
| KIT | SNV/Indel | −0.0102 | 0.9899 | 40 | 0.1055 | 0.183 | 0.9231 | 1 | 0.9743 |
| E8:WT1 | Gene:DNA methylation interaction | 0.0067 | 1.0067 | 40 | 0.0871 | 0.1996 | 0.9387 | 1 | 0.9799 |
| TET2 | SNV/Indel | −0.0074 | 0.9927 | 137 | 0.103 | 0.1476 | 0.943 | 1 | 0.9799 |
| NPM1:PTPN11 | Gene:Gene interaction | 0.0036 | 1.0036 | 64 | 0.0864 | 0.2018 | 0.9671 | 1 | 0.9909 |
| E4:GATA2 | Gene:DNA methylation interaction | 0.0016 | 1.0016 | 27 | 0.0548 | 0.2236 | 0.9765 | 1 | 0.9909 |
| EZH2 | SNV/Indel | −0.0023 | 0.9977 | 27 | 0.0978 | 0.1918 | 0.9811 | 1 | 0.9909 |
| minus Y | CNA | 0.0031 | 1.0031 | 35 | 0.1472 | 0.2337 | 0.9834 | 1 | 0.9909 |
| DNMT3A:TET2 | Gene:Gene interaction | 0.0008 | 1.0008 | 31 | 0.0932 | 0.1999 | 0.9929 | 1 | 0.9929 |

TABLE 9
Association of features with non-relapse death using multistage random effects modeling

| | | beta | | | | | | | |
|---|---|---|---|---|---|---|---|---|---|
| | | (log- | hazard | | | sd | | Q-value | Q-value |
| Feature name | Feature class | hazard) | exp(beta) | n | sd | (var) | P-value | (B-Y) | (B-H) |
| SHS | DNA methylation | 0.4584 | 1.5816 | 213 | 0.1391 | 0.2428 | 0.001 | 0.6546 | 0.1196 |
| DNMT3A:NPM1 | Gene:Gene interaction | −0.3441 | 0.7088 | 182 | 0.1102 | 0.2762 | 0.0018 | 0.6546 | 0.1196 |
| MLL | SNV/Indel | 0.3088 | 1.3618 | 14 | 0.1075 | 0.2846 | 0.0041 | 0.783 | 0.1431 |
| Age of diagnosis | Demographics | 0.2444 | 1.2769 | 1021 | 0.0871 | 0.0917 | 0.005 | 0.783 | 0.1431 |
| U2AF1 | SNV/Indel | 0.3618 | 1.4359 | 40 | 0.13 | 0.2677 | 0.0054 | 0.783 | 0.1431 |
| RAD21 | SNV/Indel | 0.3482 | 1.4166 | 24 | 0.1352 | 0.2636 | 0.01 | 1 | 0.202 |
| SRFS2 | SNV/Indel | 0.3227 | 1.3809 | 74 | 0.1321 | 0.2596 | 0.0146 | 1 | 0.202 |
| FLT3-ITD | SNV/Indel | 0.3049 | 1.3564 | 223 | 0.1253 | 0.2448 | 0.0149 | 1 | 0.202 |
| E7 | DNA methylation | 0.29 | 1.3364 | 144 | 0.1191 | 0.2648 | 0.0149 | 1 | 0.202 |
| E13 | DNA methylation | 0.5999 | 1.822 | 109 | 0.2488 | 0.3791 | 0.0159 | 1 | 0.202 |
| IDH2 p172 | SNV/Indel | 0.2882 | 1.334 | 33 | 0.1215 | 0.2778 | 0.0177 | 1 | 0.202 |
| KRAS | SNV/Indel | 0.338 | 1.4021 | 55 | 0.1432 | 0.2584 | 0.0182 | 1 | 0.202 |
| SHS:FLT3_ITD | Gene:DNA methylation | 0.2679 | 1.3072 | 112 | 0.1168 | 0.2847 | 0.0218 | 1 | 0.2189 |
| | interaction | | | | | | | | |
| SF3A1 | SNV/Indel | 0.2422 | 1.2741 | 9 | 0.1066 | 0.285 | 0.023 | 1 | 0.2189 |
| IDH1 | SNV/Indel | 0.2845 | 1.3291 | 88 | 0.1283 | 0.2541 | 0.0265 | 1 | 0.2354 |
| monosomy 7 | CNA | 0.5266 | 1.6931 | 83 | 0.2438 | 0.4054 | 0.0308 | 1 | 0.2558 |
| E8:PTPN11 | Gene:DNA methylation | 0.2362 | 1.2665 | 39 | 0.1107 | 0.2893 | 0.0328 | 1 | 0.2568 |
| | interaction | | | | | | | | |
| JAK2 | SNV/Indel | 0.2262 | 1.2538 | 5 | 0.1098 | 0.2822 | 0.0395 | 1 | 0.2915 |
| CBL | SNV/Indel | 0.2694 | 1.3092 | 21 | 0.1337 | 0.2678 | 0.044 | 1 | 0.3078 |
| IKZF1 | SNV/Indel | 0.218 | 1.2436 | 20 | 0.1119 | 0.2797 | 0.0513 | 1 | 0.3413 |
| NF1 | SNV/Indel | 0.2382 | 1.269 | 47 | 0.1247 | 0.268 | 0.0562 | 1 | 0.3524 |
| E8:NRAS | Gene:DNA methylation | −0.2237 | 0.7995 | 39 | 0.1181 | 0.2882 | 0.0583 | 1 | 0.3524 |
| | interaction | | | | | | | | |
| CEBPA-sm | SNV/Indel | 0.2508 | 1.2851 | 54 | 0.1342 | 0.2583 | 0.0616 | 1 | 0.356 |
| SHS:E7 | DNA methylation: DNA | 0.1857 | 1.204 | 65 | 0.102 | 0.2922 | 0.0688 | 1 | 0.3595 |
| | methylation | | | | | | | | |
| SF3B1 | SNV/Indel | 0.2252 | 1.2526 | 37 | 0.1247 | 0.275 | 0.071 | 1 | 0.3595 |
| GATA2:CEBPA-bi | Gene:Gene interaction | −0.1544 | 0.8569 | 27 | 0.088 | 0.3072 | 0.0792 | 1 | 0.3595 |
| E8 | DNA methylation | 0.2182 | 1.2438 | 224 | 0.1243 | 0.2627 | 0.0793 | 1 | 0.3595 |
| BRAF | SNV/Indel | 0.1942 | 1.2144 | 6 | 0.111 | 0.2778 | 0.0802 | 1 | 0.3595 |
| E5 | DNA methylation | 0.4527 | 1.5725 | 80 | 0.26 | 0.3561 | 0.0817 | 1 | 0.3595 |
| ETV6 | SNV/Indel | 0.1997 | 1.2211 | 25 | 0.1149 | 0.2769 | 0.0822 | 1 | 0.3595 |
| NPM1:FLT3- | Gene:Gene interaction | −0.1649 | 0.848 | 65 | 0.0954 | 0.2969 | 0.0838 | 1 | 0.3595 |
| ITD:DNMT3A | | | | | | | | | |
| NPM1:WT1 | Gene:Gene interaction | −0.147 | 0.8633 | 36 | 0.0874 | 0.3076 | 0.0925 | 1 | 0.3662 |
| PTPN11 | SNV/Indel | 0.2167 | 1.2419 | 101 | 0.1299 | 0.2501 | 0.0954 | 1 | 0.3662 |
| ASXL1 | SNV/Indel | 0.2164 | 1.2416 | 68 | 0.13 | 0.2527 | 0.0959 | 1 | 0.3662 |
| DNMT3A:PTPN11 | Gene:Gene interaction | −0.1807 | 0.8347 | 34 | 0.1087 | 0.2965 | 0.0966 | 1 | 0.3662 |
| SF1 | SNV/Indel | 0.1929 | 1.2127 | 9 | 0.117 | 0.2784 | 0.0991 | 1 | 0.3662 |
| PTEN | SNV/Indel | 0.1717 | 1.1873 | 5 | 0.1052 | 0.2838 | 0.1028 | 1 | 0.3694 |
| PHF6 | SNV/Indel | 0.2019 | 1.2238 | 27 | 0.1254 | 0.2685 | 0.1073 | 1 | 0.3741 |
| E4:GATA2 | Gene:DNA methylation | −0.132 | 0.8763 | 27 | 0.0825 | 0.3094 | 0.1097 | 1 | 0.3741 |
| | interaction | | | | | | | | |
| TP53 | SNV/Indel | 0.2045 | 1.2269 | 88 | 0.129 | 0.271 | 0.1128 | 1 | 0.3752 |
| NRAS:TET2 | Gene:Gene interaction | −0.168 | 0.8453 | 25 | 0.11 | 0.2968 | 0.1268 | 1 | 0.4016 |
| FLT3-TKD | SNV/Indel | 0.2034 | 1.2255 | 99 | 0.1337 | 0.2513 | 0.1282 | 1 | 0.4016 |
| E9 | DNA methylation | 0.183 | 1.2008 | 22 | 0.1208 | 0.2756 | 0.1298 | 1 | 0.4016 |
| abnormal chr 17 | CNA | −0.3083 | 0.7347 | 63 | 0.2052 | 0.4714 | 0.133 | 1 | 0.4019 |
| (monosomy 17 or | | | | | | | | | |
| del(17p) or abnormal | | | | | | | | | |
| 17p) | | | | | | | | | |
| NPM1:FLT3-ITD | Gene:Gene interaction | −0.1702 | 0.8435 | 136 | 0.1183 | 0.2709 | 0.1504 | 1 | 0.4435 |
| Platelets | Clinical | −0.1593 | 0.8528 | 1002 | 0.1116 | 0.1517 | 0.1534 | 1 | 0.4435 |
| E11 | DNA methylation | 0.1792 | 1.1963 | 55 | 0.1283 | 0.2642 | 0.1623 | 1 | 0.4538 |
| EZH2 | SNV/Indel | 0.1791 | 1.1961 | 27 | 0.1316 | 0.2698 | 0.1735 | 1 | 0.4538 |
| E8:DNMT3A | Gene:DNA methylation | −0.1445 | 0.8655 | 45 | 0.1066 | 0.298 | 0.1755 | 1 | 0.4538 |
| | interaction | | | | | | | | |
| E8:TET2 | Gene:DNA methylation | 0.1619 | 1.1757 | 34 | 0.1205 | 0.2832 | 0.179 | 1 | 0.4538 |
| | interaction | | | | | | | | |
| E10 | DNA methylation | 0.155 | 1.1677 | 40 | 0.1161 | 0.2727 | 0.1818 | 1 | 0.4538 |
| KIT | SNV/Indel | 0.1742 | 1.1903 | 40 | 0.1309 | 0.2668 | 0.1833 | 1 | 0.4538 |
| DNMT3A:FLT3-ITD | Gene: Gene interaction | −0.1366 | 0.8723 | 85 | 0.1028 | 0.2923 | 0.1837 | 1 | 0.4538 |
| RUNX1:SRFS2 | Gene:Gene interaction | 0.117 | 1.1241 | 25 | 0.0881 | 0.306 | 0.1843 | 1 | 0.4538 |
| IDH1:NPM1 | Gene:Gene interaction | 0.1545 | 1.1671 | 49 | 0.1179 | 0.2825 | 0.19 | 1 | 0.4596 |
| PB Blasts | Clinical | −0.0607 | 0.9411 | 1015 | 0.0468 | 0.0514 | 0.1947 | 1 | 0.4608 |
| RUNX1 | SNV/Indel | 0.1693 | 1.1845 | 112 | 0.1323 | 0.2485 | 0.2005 | 1 | 0.4608 |
| SHS:DNMT3A | Gene:DNA methylation | 0.1376 | 1.1475 | 88 | 0.1076 | 0.2893 | 0.201 | 1 | 0.4608 |
| | interaction | | | | | | | | |
| plus11 or +11q | CNA | 0.2112 | 1.2352 | 26 | 0.167 | 0.5268 | 0.206 | 1 | 0.461 |
| TET2 | SNV/Indel | 0.1657 | 1.1802 | 137 | 0.1316 | 0.2412 | 0.208 | 1 | 0.461 |
| t(v; 11) | Fusions | 0.3154 | 1.3708 | 39 | 0.2728 | 0.4113 | 0.2477 | 1 | 0.5322 |
| MPL | SNV/Indel | 0.1322 | 1.1414 | 11 | 0.1147 | 0.2767 | 0.249 | 1 | 0.5322 |
| WT1:FLT3-ITD | Gene:Gene interaction | −0.0872 | 0.9165 | 31 | 0.0761 | 0.3127 | 0.2521 | 1 | 0.5322 |
| BCOR | SNV/Indel | 0.1501 | 1.1619 | 63 | 0.1359 | 0.2542 | 0.2696 | 1 | 0.5603 |
| CEBPA-dm | SNV/Indel | 0.1386 | 1.1487 | 72 | 0.1288 | 0.2697 | 0.2818 | 1 | 0.5725 |
| monosomy 18 or | CNA | 0.1906 | 1.2099 | 23 | 0.1805 | 0.5222 | 0.291 | 1 | 0.5725 |
| del(18q) | | | | | | | | | |
| E12 | DNA methylation | −0.2684 | 0.7646 | 180 | 0.2547 | 0.4013 | 0.2919 | 1 | 0.5725 |
| STAG2 | SNV/Indel | 0.1323 | 1.1414 | 30 | 0.1258 | 0.2695 | 0.2932 | 1 | 0.5725 |
| DNMT3A | SNVAIndel | 0.1365 | 1.1463 | 266 | 0.1309 | 0.2445 | 0.297 | 1 | 0.5725 |
| DNMT3A:TET2 | Gene:Gene interaction | −0.1103 | 0.8956 | 31 | 0.107 | 0.2998 | 0.3027 | 1 | 0.5752 |
| HB | Clinical | −0.0768 | 0.926 | 987 | 0.0759 | 0.2111 | 0.3113 | 1 | 0.5831 |
| E8:FLT3_TKD | Gene:DNA methylation | −0.1091 | 0.8966 | 36 | 0.1091 | 0.2947 | 0.3173 | 1 | 0.5862 |
| | interaction | | | | | | | | |
| Splenomegaly | Clinical | −0.0495 | 0.9517 | 69 | 0.0509 | 0.2203 | 0.33 | 1 | 0.593 |
| tAML | Clinical | −0.0495 | 0.9517 | 7 | 0.0509 | 0.2203 | 0.33 | 1 | 0.593 |
| monosomy 20 or | CNA | −0.2163 | 0.8055 | 28 | 0.2278 | 0.4864 | 0.3422 | 1 | 0.6068 |
| del(20q) | | | | | | | | | |
| E8:FLT3_ITD | Gene:DNA methylation | −0.1098 | 0.896 | 77 | 0.1202 | 0.2847 | 0.3612 | 1 | 0.624 |
| | interaction | | | | | | | | |
| E4:CEBPA_bi | Gene:DNA methylation | −0.0894 | 0.9145 | 57 | 0.0979 | 0.2964 | 0.3613 | 1 | 0.624 |
| | interaction | | | | | | | | |
| NPM1:FLT3-TKD | Gene:Gene interaction | −0.1013 | 0.9036 | 60 | 0.1149 | 0.2805 | 0.3777 | 1 | 0.6441 |
| plus21 | CNA | −0.1982 | 0.8202 | 25 | 0.2286 | 0.4914 | 0.3859 | 1 | 0.6497 |
| del(7q) | CNA | −0.2099 | 0.8107 | 53 | 0.2469 | 0.4467 | 0.3952 | 1 | 0.657 |
| TET2:FLT3-ITD | Gene:Gene interaction | 0.0917 | 1.096 | 34 | 0.1163 | 0.2903 | 0.4307 | 1 | 0.7072 |
| complex karyotype | CNA | 0.1706 | 1.186 | 121 | 0.2206 | 0.4134 | 0.4395 | 1 | 0.7129 |
| NPM1:IDH2 p140 | Gene:Gene interaction | −0.0886 | 0.9152 | 47 | 0.1185 | 0.2873 | 0.4545 | 1 | 0.7283 |
| NPM1 | SNV/Indel | 0.0947 | 1.0993 | 382 | 0.13 | 0.2504 | 0.4666 | 1 | 0.7387 |
| t(8; 21) | Fusions | 0.1809 | 1.1983 | 22 | 0.2608 | 0.4121 | 0.488 | 1 | 0.7438 |
| minusY | CNA | −0.1756 | 0.8389 | 35 | 0.2539 | 0.4571 | 0.4891 | 1 | 0.7438 |
| NRAS | SNV/Indel | 0.0932 | 1.0977 | 167 | 0.1356 | 0.2349 | 0.4918 | 1 | 0.7438 |
| LDH | Clinical | −0.0382 | 0.9625 | 759 | 0.0556 | 0.2185 | 0.4921 | 1 | 0.7438 |
| E11:NPM1 | Gene:DNA methylation | −0.067 | 0.9352 | 36 | 0.1 | 0.2953 | 0.5028 | 1 | 0.7513 |
| | interaction | | | | | | | | |
| E3 | DNA methylation | 0.1679 | 1.1828 | 26 | 0.2588 | 0.4126 | 0.5164 | 1 | 0.7566 |
| t(9; 11) | Fusions | 0.1699 | 1.1852 | 24 | 0.2657 | 0.3925 | 0.5226 | 1 | 0.7566 |
| t(6; 9) | Fusions | 0.1529 | 1.1652 | 6 | 0.2403 | 0.4584 | 0.5247 | 1 | 0.7566 |
| inv(16) | Fusions | 0.1631 | 1.1772 | 24 | 0.2592 | 0.4104 | 0.529 | 1 | 0.7566 |
| del(9q) | CNA | −0.13 | 0.8781 | 15 | 0.21 | 0.5036 | 0.5361 | 1 | 0.7585 |
| abnormal chr 3q | CNA | −0.1221 | 0.8851 | 12 | 0.2035 | 0.5054 | 0.5484 | 1 | 0.7678 |
| ZRSR2 | SNV/Indel | 0.0757 | 1.0787 | 49 | 0.1338 | 0.2621 | 0.5715 | 1 | 0.782 |
| E7:NPM1 | Gene:DNA methylation | 0.0569 | 1.0586 | 130 | 0.1029 | 0.2823 | 0.5801 | 1 | 0.782 |
| | interaction | | | | | | | | |
| DNMT3A:FLT3- | Gene:Gene interaction | 0.0575 | 1.0591 | 31 | 0.1043 | 0.2989 | 0.5819 | 1 | 0.782 |
| TKD | | | | | | | | | |
| abnormal chr 7 | CNA | 0.1183 | 1.1256 | 12 | 0.215 | 0.5036 | 0.5821 | 1 | 0.782 |
| (other) | | | | | | | | | |
| abnormal chr 4 | CNA | −0.1032 | 0.902 | 17 | 0.1916 | 0.5066 | 0.5901 | 1 | 0.7849 |
| (monosomy 4 or | | | | | | | | | |
| del(4p) or abnormal | | | | | | | | | |
| 4p) | | | | | | | | | |
| NPM1:TET2 | Gene:Gene interaction | 0.0614 | 1.0634 | 64 | 0.1192 | 0.2762 | 0.6062 | 1 | 0.7983 |
| BM Blasts | Clinical | −0.0299 | 0.9705 | 1014 | 0.0607 | 0.0674 | 0.6223 | 1 | 0.8048 |
| SRFS2:IDH2_p140 | Gene:Gene interaction | −0.0479 | 0.9532 | 29 | 0.0975 | 0.3011 | 0.6233 | 1 | 0.8048 |
| E8:WT1 | Gene:DNA methylation | −0.0426 | 0.9583 | 40 | 0.0887 | 0.3072 | 0.6309 | 1 | 0.8068 |
| | interaction | | | | | | | | |
| E2 | DNA methylation | 0.1244 | 1.1325 | 29 | 0.2638 | 0.4071 | 0.6372 | 1 | 0.8071 |
| GATA2 | SNV/Indel | 0.0566 | 1.0583 | 57 | 0.1315 | 0.2666 | 0.6668 | 1 | 0.8264 |
| plus8 or +8q | CNA | −0.1127 | 0.8934 | 104 | 0.2634 | 0.363 | 0.6688 | 1 | 0.8264 |
| E7:FLT3_ITD | Gene:DNA methylation | −0.0428 | 0.9581 | 56 | 0.1009 | 0.2957 | 0.6711 | 1 | 0.8264 |
| | interaction | | | | | | | | |
| E4 | DNA methylation | −0.1038 | 0.9014 | 74 | 0.2722 | 0.4118 | 0.7029 | 1 | 0.8577 |
| NPM1:NRAS | Gene:Gene interaction | 0.0455 | 1.0466 | 67 | 0.1223 | 0.2745 | 0.7099 | 1 | 0.8584 |
| monosomy 5 or | CNA | 0.0762 | 1.0792 | 83 | 0.2102 | 0.4557 | 0.7169 | 1 | 0.859 |
| del(5q) | | | | | | | | | |
| ECOG Performance | Clinical | 0.036 | 1.0366 | 880 | 0.1071 | 0.1413 | 0.737 | 1 | 0.8727 |
| status | | | | | | | | | |
| Gender | Demographics | 0.0573 | 1.059 | 1021 | 0.1766 | 0.2131 | 0.7454 | 1 | 0.8727 |
| DNMT3A:IDH1 | Gene:Gene interaction | −0.0353 | 0.9653 | 42 | 0.1099 | 0.2973 | 0.748 | 1 | 0.8727 |
| abnormal chr 12 | CNA | 0.0721 | 1.0748 | 40 | 0.236 | 0.4518 | 0.76 | 1 | 0.8789 |
| (monosomy 12 or | | | | | | | | | |
| del(12p) or abnormal | | | | | | | | | |
| 12p) | | | | | | | | | |
| E8:NPM1 | Gene:DNA methylation | 0.033 | 1.0336 | 156 | 0.1138 | 0.2677 | 0.7717 | 1 | 0.8848 |
| | interaction | | | | | | | | |
| ASXL1:RUNX1 | Gene:Gene interaction | 0.0264 | 1.0267 | 25 | 0.1013 | 0.2996 | 0.7949 | 1 | 0.9036 |
| DNMT3A:IDH2 | Gene:Gene interaction | −0.0219 | 0.9784 | 33 | 0.0914 | 0.3077 | 0.8109 | 1 | 0.9103 |
| p140 | | | | | | | | | |
| WT1 | SNV/Indel | 0.0303 | 1.0308 | 86 | 0.1316 | 0.2632 | 0.818 | 1 | 0.9103 |
| plus22 | CNA | −0.0524 | 0.9489 | 25 | 0.232 | 0.4745 | 0.8213 | 1 | 0.9103 |
| NPM1:CEBPA-mono | Gene:Gene interaction | 0.022 | 1.0222 | 27 | 0.1132 | 0.2941 | 0.8462 | 1 | 0.9302 |
| DNMT3A.NRAS | Gene:Gene interaction | −0.0206 | 0.9796 | 47 | 0.1131 | 0.2933 | 0.8552 | 1 | 0.9323 |
| WBC | Clinical | −0.0178 | 0.9823 | 1007 | 0.1032 | 0.1826 | 0.8627 | 1 | 0.9328 |
| E7:DNMT3A | Gene:DNA methylation | −0.0143 | 0.9858 | 119 | 0.1027 | 0.2857 | 0.8889 | 1 | 0.9534 |
| | interaction | | | | | | | | |
| SHS:NPM1 | Gene:DNA methylation | 0.0135 | 1.0135 | 117 | 0.1164 | 0.2798 | 0.908 | 1 | 0.9661 |
| | interaction | | | | | | | | |
| E6 | DNA methylation | 0.0241 | 1.0244 | 38 | 0.2615 | 0.4289 | 0.9266 | 1 | 0.9781 |
| ASXL1:SRFS2 | Gene:Gene interaction | −0.0079 | 0.9921 | 25 | 0.0971 | 0.3026 | 0.9349 | 1 | 0.979 |
| NF1:NPM1 | Gene:Gene interaction | 0.007 | 1.007 | 18 | 0.1012 | 0.3019 | 0.9448 | 1 | 0.9817 |
| plus13 | CNA | −0.0142 | 0.9859 | 23 | 0.2371 | 0.483 | 0.9524 | 1 | 0.9819 |
| IDH2 p140 | SNV/Indel | −0.0045 | 0.9955 | 91 | 0.1313 | 0.2497 | 0.9727 | 1 | 0.9951 |
| SHS:WT1 | Gene:DNA methylation | −0.0005 | 0.9995 | 32 | 0.0821 | 0.3107 | 0.9947 | 1 | 0.9995 |
| | interaction | | | | | | | | |
| SHS:E8 | DNA methylation:DNA | 0.0007 | 1.0007 | 71 | 0.1109 | 0.2946 | 0.9949 | 1 | 0.9995 |
| | methylation | | | | | | | | |
| NPM1:PTPN11 | Gene:Gene interaction | −0.0001 | 0.9999 | 64 | 0.1113 | 0.2804 | 0.9995 | 1 | 0.9995 |

TABLE 10
Association of features with relapse using multistage random effects modeling

| | | beta | | | | | | | |
|---|---|---|---|---|---|---|---|---|---|
| | | (log- | hazard | | | sd | | Q-value | Q-value |
| Feature name | Feature class | hazard) | exp(beta) | n | sd | (var) | P-value | (B-Y) | (B-H) |
| E12 | DNA methylation | 0.3849 | 1.4695 | 180 | 0.0946 | 0.16 | 0 | 0.0097 | 0.0018 |
| del(9q) | CNA | 0.4238 | 1.5278 | 15 | 0.1019 | 0.1981 | 0 | 0.0097 | 0.0018 |
| WBC | Clinical | 0.3624 | 1.4368 | 1007 | 0.0872 | 0.099 | 0 | 0.0097 | 0.0018 |
| plus11 or +11q | CNA | 0.2923 | 1.3395 | 26 | 0.0723 | 0.2204 | 0.0001 | 0.0097 | 0.0018 |
| E4 | DNA methylation | −0.8319 | 0.4352 | 74 | 0.2254 | 0.2989 | 0.0002 | 0.0211 | 0.0039 |
| monosomy 18 or | CNA | 0.2774 | 1.3196 | 23 | 0.0753 | 0.2187 | 0.0002 | 0.0211 | 0.0039 |
| del(18q) | | | | | | | | | |
| abnormal chr 17 | CNA | 0.3288 | 1.3893 | 63 | 0.0892 | 0.1897 | 0.0002 | 0.0211 | 0.0039 |
| (monosomy 17 or | | | | | | | | | |
| del(17p) or abnormal | | | | | | | | | |
| 17p) | | | | | | | | | |
| abnormal chr 4 | CNA | 0.3132 | 1.3679 | 17 | 0.0851 | 0.2088 | 0.0002 | 0.0211 | 0.0039 |
| (monosomy 4 or del(4p) | | | | | | | | | |
| or abnormal 4p) | | | | | | | | | |
| monosomy 5 or del(5q) | CNA | 0.3233 | 1.3817 | 83 | 0.09 | 0.1818 | 0.0003 | 0.0266 | 0.0049 |
| E6 | DNA methylation | 0.8374 | 2.3102 | 38 | 0.2399 | 0.2928 | 0.0005 | 0.0348 | 0.0064 |
| NPM1 | SNV/Indel | −0.3066 | 0.7359 | 382 | 0.0884 | 0.1605 | 0.0005 | 0.0348 | 0.0064 |
| E7: FLT3_ITD | Gene: DNA methylation | 0.3653 | 1.441 | 56 | 0.1082 | 0.2195 | 0.0007 | 0.0448 | 0.0082 |
| | interaction | | | | | | | | |
| SHS: NPM1 | Gene: DNA methylation | 0.3552 | 1.4264 | 117 | 0.1136 | 0.1985 | 0.0018 | 0.099 | 0.0181 |
| | interaction | | | | | | | | |
| t(9; 11) | Fusions | −0.8825 | 0.4138 | 24 | 0.2865 | 0.3324 | 0.0021 | 0.1075 | 0.0196 |
| Age of diagnosis | Demographics | 0.1049 | 1.1106 | 1021 | 0.0366 | 0.0383 | 0.0041 | 0.2 | 0.0366 |
| Splenomegaly | Clinical | 0.0934 | 1.0979 | 69 | 0.0336 | 0.2397 | 0.0055 | 0.233 | 0.0426 |
| tAML | Clinical | 0.0934 | 1.0979 | 7 | 0.0336 | 0.2397 | 0.0055 | 0.233 | 0.0426 |
| LDH | Clinical | 0.1998 | 1.2211 | 759 | 0.0724 | 0.2281 | 0.0058 | 0.233 | 0.0426 |
| complex karyotype | CNA | 0.2512 | 1.2856 | 121 | 0.0921 | 0.1608 | 0.0064 | 0.2437 | 0.0445 |
| DNMT3A: FLT3-ITD | Gene: Gene interaction | 0.278 | 1.3205 | 85 | 0.1025 | 0.2153 | 0.0067 | 0.244 | 0.0446 |
| abnormal chr 7 (other) | CNA | 0.2208 | 1.2471 | 12 | 0.0894 | 0.2105 | 0.0135 | 0.4688 | 0.0857 |
| E8: TET2 | Gene: DNA methylation | −0.3015 | 0.7397 | 34 | 0.123 | 0.2098 | 0.0142 | 0.4708 | 0.086 |
| | interaction | | | | | | | | |
| SHS | DNA methylation | 0.2354 | 1.2654 | 213 | 0.0972 | 0.1594 | 0.0154 | 0.4884 | 0.0893 |
| inv(16) | Fusions | −0.6479 | 0.5231 | 24 | 0.2754 | 0.4487 | 0.0186 | 0.5457 | 0.0997 |
| minusY | CNA | 0.2531 | 1.288 | 35 | 0.1077 | 0.189 | 0.0187 | 0.5457 | 0.0997 |
| WT1: FLT3-ITD | Gene: Gene interaction | 0.2697 | 1.3095 | 31 | 0.1157 | 0.2269 | 0.0198 | 0.5535 | 0.1012 |
| t(8; 21) | Fusions | −0.7264 | 0.4837 | 22 | 0.3153 | 0.4388 | 0.0212 | 0.5643 | 0.1031 |
| E8: NPM1 | Gene: DNA methylation | −0.2369 | 0.7891 | 156 | 0.1032 | 0.1892 | 0.0217 | 0.5643 | 0.1031 |
| | interaction | | | | | | | | |
| SHS: E8 | DNA methylation: DNA | 0.2615 | 1.2988 | 71 | 0.1146 | 0.2041 | 0.0225 | 0.5643 | 0.1031 |
| | methylation | | | | | | | | |
| monosomy 7 | CNA | 0.2312 | 1.2601 | 83 | 0.1043 | 0.175 | 0.0266 | 0.6459 | 0.1181 |
| NPM1: FLT3-TKD | Gene: Gene interaction | −0.2338 | 0.7915 | 60 | 0.1065 | 0.2067 | 0.0282 | 0.6608 | 0.1208 |
| plus8 or +8q | CNA | 0.2251 | 1.2524 | 104 | 0.1086 | 0.1533 | 0.0383 | 0.8715 | 0.1593 |
| E7 | DNA methylation | −0.1598 | 0.8523 | 144 | 0.0814 | 0.188 | 0.0496 | 1 | 0.1998 |
| DNMT3A: IDH1 | Gene: Gene interaction | 0.2312 | 1.2602 | 42 | 0.1194 | 0.2104 | 0.0527 | 1 | 0.2063 |
| abnormal chr 3q | CNA | 0.1784 | 1.1953 | 12 | 0.0942 | 0.1982 | 0.0581 | 1 | 0.2209 |
| IDH1 | SNV/Indel | 0.1645 | 1.1788 | 88 | 0.0892 | 0.1726 | 0.0651 | 1 | 0.2404 |
| DNMT3A: TET2 | Gene: Gene interaction | 0.215 | 1.2399 | 31 | 0.1218 | 0.2171 | 0.0775 | 1 | 0.2786 |
| E13 | DNA methylation | 0.1798 | 1.197 | 109 | 0.103 | 0.1725 | 0.0808 | 1 | 0.2828 |
| plus21 | CNA | 0.1813 | 1.1988 | 25 | 0.1079 | 0.1872 | 0.0929 | 1 | 0.3106 |
| MLL | SNV/Indel | 0.1363 | 1.1461 | 14 | 0.0813 | 0.2074 | 0.0934 | 1 | 0.3106 |
| RUNX1 | SNV/Indel | 0.1634 | 1.1775 | 112 | 0.0994 | 0.1533 | 0.1002 | 1 | 0.3252 |
| WT1 | SNV/Indel | 0.1623 | 1.1762 | 86 | 0.1002 | 0.165 | 0.1053 | 1 | 0.3334 |
| SF1 | SNV/Indel | −0.1439 | 0.866 | 9 | 0.0904 | 0.1996 | 0.1116 | 1 | 0.3452 |
| SHS: WT1 | Gene: DNA methylation | 0.1782 | 1.195 | 32 | 0.1144 | 0.2295 | 0.1192 | 1 | 0.3536 |
| | interaction | | | | | | | | |
| BCOR | SNV/Indel | −0.1645 | 0.8483 | 63 | 0.1057 | 0.1606 | 0.1196 | 1 | 0.3536 |
| NPM1: FLT3-ITD | Gene: Gene interaction | −0.1671 | 0.8461 | 136 | 0.1113 | 0.1905 | 0.1331 | 1 | 0.3847 |
| ASXL1: RUNX1 | Gene: Gene interaction | −0.1627 | 0.8499 | 25 | 0.1105 | 0.2274 | 0.141 | 1 | 0.3916 |
| MPL | SNV/Indel | 0.1252 | 1.1334 | 11 | 0.0851 | 0.202 | 0.1413 | 1 | 0.3916 |
| NPM1: WT1 | Gene: Gene interaction | 0.1651 | 1.1795 | 36 | 0.1137 | 0.2203 | 0.1463 | 1 | 0.3971 |
| E8: NRAS | Gene: DNA methylation | 0.1744 | 1.1906 | 39 | 0.1229 | 0.2044 | 0.1558 | 1 | 0.4088 |
| | interaction | | | | | | | | |
| SF3B1 | SNV/Indel | 0.1499 | 1.1617 | 37 | 0.1058 | 0.1822 | 0.1567 | 1 | 0.4088 |
| ECOG Performance | Clinical | 0.0911 | 1.0953 | 880 | 0.0671 | 0.0727 | 0.175 | 1 | 0.4416 |
| status | | | | | | | | | |
| Platelets | Clinical | −0.0951 | 0.9093 | 1002 | 0.0704 | 0.077 | 0.1767 | 1 | 0.4416 |
| t(v; 11) | Fusions | 0.3322 | 1.394 | 39 | 0.2473 | 0.2777 | 0.1793 | 1 | 0.4416 |
| E8: WT1 | Gene: DNA methylation | 0.1489 | 1.1605 | 40 | 0.1119 | 0.2164 | 0.1832 | 1 | 0.4429 |
| | interaction | | | | | | | | |
| E8: FLT3_ITD | Gene: DNA methylation | 0.157 | 1.17 | 77 | 0.1205 | 0.2004 | 0.1928 | 1 | 0.4579 |
| | interaction | | | | | | | | |
| ZRSR2 | SNV/Indel | 0.1366 | 1.1463 | 49 | 0.107 | 0.1637 | 0.202 | 1 | 0.4713 |
| E8: FLT3_TKD | Gene: DNA methylation | 0.1468 | 1.1581 | 36 | 0.1187 | 0.2122 | 0.2162 | 1 | 0.4825 |
| | interaction | | | | | | | | |
| E8 | DNA methylation | 0.1083 | 1.1144 | 224 | 0.0878 | 0.1764 | 0.2173 | 1 | 0.4825 |
| E7: NPM1 | Gene: DNA methylation | −0.1215 | 0.8856 | 130 | 0.099 | 0.2145 | 0.2198 | 1 | 0.4825 |
| | interaction | | | | | | | | |
| PB Blasts | Clinical | 0.0265 | 1.0268 | 1015 | 0.0219 | 0.023 | 0.2267 | 1 | 0.4825 |
| abnormal chr 12 | CNA | 0.1259 | 1.1341 | 40 | 0.1051 | 0.1824 | 0.2311 | 1 | 0.4825 |
| (monosomy 12 or | | | | | | | | | |
| del(12p) or abnormal | | | | | | | | | |
| 12p) | | | | | | | | | |
| TET2 | SNV/Indel | 0.1101 | 1.1164 | 137 | 0.0921 | 0.1585 | 0.2319 | 1 | 0.4825 |
| del(7q) | CNA | 0.1258 | 1.134 | 53 | 0.1053 | 0.1734 | 0.2322 | 1 | 0.4825 |
| IDH2 p172 | SNV/Indel | −0.1182 | 0.8885 | 33 | 0.1006 | 0.1839 | 0.2402 | 1 | 0.4914 |
| KRAS | SNV/Indel | −0.1241 | 0.8833 | 55 | 0.1116 | 0.1606 | 0.2662 | 1 | 0.5365 |
| plus13 | CNA | 0.1128 | 1.1194 | 23 | 0.1031 | 0.198 | 0.2738 | 1 | 0.5436 |
| BRAF | SNV/Indel | −0.0864 | 0.9172 | 6 | 0.0808 | 0.205 | 0.2846 | 1 | 0.5516 |
| monosomy 20 or | CNA | 0.1052 | 1.111 | 28 | 0.0987 | 0.1888 | 0.2862 | 1 | 0.5516 |
| del(20g) | | | | | | | | | |
| FLT3-ITD | SNV/Indel | 0.0976 | 1.1025 | 223 | 0.095 | 0.1605 | 0.3042 | 1 | 0.578 |
| E4: GATA2 | Gene: DNA methylation | 0.1059 | 1.1117 | 27 | 0.1059 | 0.2348 | 0.3174 | 1 | 0.5945 |
| | interaction | | | | | | | | |
| TP53 | SNV/Indel | 0.0981 | 1.1031 | 88 | 0.1066 | 0.1777 | 0.3573 | 1 | 0.6601 |
| E2 | DNA methylation | −0.2493 | 0.7793 | 29 | 0.2828 | 0.3855 | 0.3779 | 1 | 0.6885 |
| SHS: DNMT3A | Gene: DNA methylation | −0.094 | 0.9103 | 88 | 0.1156 | 0.2065 | 0.416 | 1 | 0.7477 |
| | interaction | | | | | | | | |
| E8: DNMT3A | Gene: DNA methylation | 0.0927 | 1.0971 | 45 | 0.1176 | 0.2047 | 0.4306 | 1 | 0.7556 |
| | interaction | | | | | | | | |
| E11 | DNA methylation | −0.0744 | 0.9283 | 55 | 0.0947 | 0.1696 | 0.4318 | 1 | 0.7556 |
| plus22 | CNA | 0.0772 | 1.0802 | 25 | 0.1022 | 0.1899 | 0.4503 | 1 | 0.7768 |
| FLT3-TKD | SNV/Indel | −0.0733 | 0.9293 | 99 | 0.0986 | 0.1621 | 0.4569 | 1 | 0.7768 |
| E3 | DNA methylation | 0.1951 | 1.2154 | 26 | 0.2649 | 0.4376 | 0.4614 | 1 | 0.7768 |
| NPM1: TET2 | Gene: Gene interaction | −0.0823 | 0.921 | 64 | 0.1156 | 0.1999 | 0.4766 | 1 | 0.7838 |
| NRAS: TET2 | Gene: Gene interaction | −0.089 | 0.9148 | 25 | 0.1253 | 0.2088 | 0.4774 | 1 | 0.7838 |
| HB | Clinical | 0.0792 | 1.0825 | 987 | 0.1176 | 0.1809 | 0.5004 | 1 | 0.8116 |
| ETV6 | SNV/Indel | 0.0628 | 1.0648 | 25 | 0.0977 | 0.1867 | 0.5206 | 1 | 0.8191 |
| E11: NPM1 | Gene: DNA methylation | −0.0605 | 0.9413 | 36 | 0.0951 | 0.2242 | 0.5251 | 1 | 0.8191 |
| | interaction | | | | | | | | |
| DNMT3A | SNV/Indel | 0.059 | 1.0607 | 266 | 0.0934 | 0.1534 | 0.5279 | 1 | 0.8191 |
| DNMT3A: PTPN11 | Gene: Gene interaction | 0.0725 | 1.0751 | 34 | 0.1169 | 0.2169 | 0.5354 | 1 | 0.8191 |
| NPM1: NRAS | Gene: Gene interaction | −0.071 | 0.9314 | 67 | 0.1147 | 0.1955 | 0.5358 | 1 | 0.8191 |
| IDH1: NPM1 | Gene: Gene interaction | −0.0694 | 0.9329 | 49 | 0.1142 | 0.2064 | 0.5432 | 1 | 0.821 |
| DNMT3A: FLT3-TKD | Gene: Gene interaction | −0.0671 | 0.9351 | 31 | 0.1163 | 0.223 | 0.564 | 1 | 0.8428 |
| STAG2 | SNV/Indel | 0.0531 | 1.0546 | 30 | 0.1019 | 0.1829 | 0.6019 | 1 | 0.8585 |
| IDH2 p140 | SNV/Indel | 0.0455 | 1.0466 | 91 | 0.0874 | 0.1665 | 0.6023 | 1 | 0.8585 |
| NPM1: PTPN11 | Gene: Gene interaction | 0.05 | 1.0513 | 64 | 0.0965 | 0.213 | 0.6046 | 1 | 0.8585 |
| SHS: FLT3_ITD | Gene: DNA methylation | 0.063 | 1.065 | 112 | 0.1224 | 0.1961 | 0.6067 | 1 | 0.8585 |
| | interaction | | | | | | | | |
| JAK2 | SNV/Indel | −0.0401 | 0.9607 | 5 | 0.078 | 0.2087 | 0.6067 | 1 | 0.8585 |
| CBL | SNV/Indel | −0.0483 | 0.9528 | 21 | 0.0985 | 0.1945 | 0.6238 | 1 | 0.8596 |
| SRFS2: IDH2_p140 | Gene: Gene interaction | 0.0524 | 1.0538 | 29 | 0.1095 | 0.227 | 0.6326 | 1 | 0.8596 |
| SHS: E7 | DNA methylation: DNA | 0.0512 | 1.0525 | 65 | 0.1075 | 0.2175 | 0.634 | 1 | 0.8596 |
| | methylation | | | | | | | | |
| t(6; 9) | Fusions | 0.1333 | 1.1426 | 6 | 0.283 | 0.6833 | 0.6377 | 1 | 0.8596 |
| DNMT3A: IDH2 p140 | Gene: Gene interaction | 0.0551 | 1.0566 | 33 | 0.119 | 0.2218 | 0.6435 | 1 | 0.8596 |
| E10 | DNA methylation | −0.0344 | 0.9662 | 40 | 0.075 | 0.1957 | 0.6463 | 1 | 0.8596 |
| Gender | Demographics | −0.0398 | 0.961 | 1021 | 0.0931 | 0.0996 | 0.6691 | 1 | 0.881 |
| PHF6 | SNV/Indel | −0.0411 | 0.9598 | 27 | 0.1028 | 0.179 | 0.6895 | 1 | 0.8963 |
| NPM1: CEBPA-mono | Gene: Gene interaction | 0.046 | 1.0471 | 27 | 0.117 | 0.2142 | 0.6941 | 1 | 0.8963 |
| E8: PTPN11 | Gene: DNA methylation | −0.0447 | 0.9562 | 39 | 0.1165 | 0.2167 | 0.7009 | 1 | 0.8964 |
| | interaction | | | | | | | | |
| CEBPA-dm | SNV/Indel | −0.0352 | 0.9654 | 72 | 0.0974 | 0.1799 | 0.7177 | 1 | 0.9008 |
| CEBPA-sm | SNV/Indel | −0.0352 | 0.9654 | 54 | 0.0992 | 0.1716 | 0.7225 | 1 | 0.9008 |
| BM Blasts | Clinical | −0.0103 | 0.9897 | 1014 | 0.0294 | 0.0309 | 0.7247 | 1 | 0.9008 |
| TET2: FLT3-ITD | Gene: Gene interaction | −0.042 | 0.9589 | 34 | 0.1244 | 0.2123 | 0.7356 | 1 | 0.9059 |
| ASXL1: SRFS2 | Gene: Gene interaction | −0.0338 | 0.9667 | 25 | 0.1069 | 0.2378 | 0.7517 | 1 | 0.9116 |
| EZH2 | SNV/Indel | −0.0337 | 0.9669 | 27 | 0.1075 | 0.1787 | 0.7539 | 1 | 0.9116 |
| RAD21 | SNV/Indel | −0.0323 | 0.9683 | 24 | 0.1074 | 0.1798 | 0.764 | 1 | 0.9154 |
| KIT | SNV/Indel | 0.0284 | 1.0288 | 40 | 0.1087 | 0.1743 | 0.7936 | 1 | 0.9268 |
| PTEN | SNV/Indel | −0.0167 | 0.9834 | 5 | 0.0644 | 0.214 | 0.7954 | 1 | 0.9268 |
| RUNX1: SRFS2 | Gene: Gene interaction | 0.0268 | 1.0271 | 25 | 0.1038 | 0.2399 | 0.7964 | 1 | 0.9268 |
| E7: DNMT3A | Gene: DNA methylation | −0.0265 | 0.9738 | 119 | 0.1055 | 0.213 | 0.8014 | 1 | 0.9268 |
| | interaction | | | | | | | | |
| E9 | DNA methylation | 0.0207 | 1.021 | 22 | 0.09 | 0.1944 | 0.8177 | 1 | 0.9375 |
| NRAS | SNV/Indel | 0.018 | 1.0182 | 167 | 0.097 | 0.1451 | 0.8526 | 1 | 0.9692 |
| GATA2 | SNV/Indel | 0.0168 | 1.0169 | 57 | 0.0999 | 0.1738 | 0.8668 | 1 | 0.9723 |
| NPM1: FLT3- | Gene: Gene interaction | 0.0137 | 1.0138 | 65 | 0.0939 | 0.2248 | 0.8842 | 1 | 0.9723 |
| ITD: DNMT3A | | | | | | | | | |
| SF3A1 | SNV/Indel | −0.0106 | 0.9895 | 9 | 0.076 | 0.2108 | 0.8895 | 1 | 0.9723 |
| NPM1: IDH2 p140 | Gene: Gene interaction | −0.0148 | 0.9853 | 47 | 0.1127 | 0.2007 | 0.8952 | 1 | 0.9723 |
| NF1: NPM1 | Gene: Gene interaction | −0.0146 | 0.9855 | 18 | 0.1107 | 0.2328 | 0.8953 | 1 | 0.9723 |
| E4: CEBPA_bi | Gene: DNA methylation | −0.0132 | 0.9869 | 57 | 0.1065 | 0.2333 | 0.9012 | 1 | 0.9723 |
| | interaction | | | | | | | | |
| GATA2: CEBPA-bi | Gene: Gene interaction | 0.0125 | 1.0125 | 27 | 0.1061 | 0.2282 | 0.9065 | 1 | 0.9723 |
| IKZF1 | SNV/Indel | −0.0086 | 0.9914 | 20 | 0.0921 | 0.193 | 0.9254 | 1 | 0.9846 |
| E5 | DNA methylation | 0.0158 | 1.0159 | 80 | 0.1914 | 0.2397 | 0.9343 | 1 | 0.9862 |
| DNMT3A: NRAS | Gene: Gene interaction | −0.006 | 0.994 | 47 | 0.1217 | 0.2104 | 0.9605 | 1 | 0.9901 |
| DNMT3A: NPM1 | Gene: Gene interaction | 0.0046 | 1.0046 | 182 | 0.105 | 0.1959 | 0.9653 | 1 | 0.9901 |
| U2AF1 | SNV/Indel | −0.0043 | 0.9957 | 40 | 0.1006 | 0.187 | 0.9659 | 1 | 0.9901 |
| NF1 | SNV/Indel | −0.0038 | 0.9962 | 47 | 0.0946 | 0.1835 | 0.9678 | 1 | 0.9901 |
| PTPN11 | SNV/Indel | 0.0024 | 1.0024 | 101 | 0.092 | 0.1725 | 0.9789 | 1 | 0.9938 |
| SRFS2 | SNV/Indel | −0.0008 | 0.9992 | 74 | 0.0992 | 0.1758 | 0.9932 | 1 | 0.994 |
| ASXL1 | SNV/Indel | 0.0007 | 1.0007 | 68 | 0.0958 | 0.1736 | 0.994 | 1 | 0.994 |

TABLE 11
Association of features with post-relapse death using multistage random effects modeling

| | | beta | | | | | | | |
|---|---|---|---|---|---|---|---|---|---|
| | | (log- | hazard | | | sd | | Q-value | Q-value |
| Feature name | Feature class | hazard) | exp(beta) | n | sd | (var) | P-value | (B-Y) | (B-H) |
| Age of diagnosis | Demographics | 0.1956 | 1.2161 | 1021 | 0.0398 | 0.0422 | 0 | 0.0006 | 0.0001 |
| E7 | DNA methylation | 0.3214 | 1.379 | 144 | 0.0692 | 0.1937 | 0 | 0.0012 | 0.0002 |
| plusil or +11q | CNA | 0.1984 | 1.2194 | 26 | 0.0577 | 0.1886 | 0.0006 | 0.1421 | 0.026 |
| CBL | SNV/Indel | 0.2449 | 1.2775 | 21 | 0.073 | 0.2109 | 0.0008 | 0.1455 | 0.0266 |
| IDH1 | SNV/Indel | 0.2868 | 1.3321 | 88 | 0.09 | 0.173 | 0.0014 | 0.2092 | 0.0382 |
| E6 | DNA methylation | 0.5577 | 1.7467 | 38 | 0.1944 | 0.2632 | 0.0041 | 0.4869 | 0.089 |
| abnormal chr 17 | CNA | 0.2066 | 1.2294 | 63 | 0.0754 | 0.1632 | 0.0062 | 0.4869 | 0.089 |
| (monosomy | | | | | | | | | |
| 17 or del(17p) | | | | | | | | | |
| or abnormal | | | | | | | | | |
| 17p) | | | | | | | | | |
| E8: FLT3_ITD | Gene: DNA methylation | 0.2339 | 1.2635 | 77 | 0.0859 | 0.1798 | 0.0064 | 0.4869 | 0.089 |
| | interaction | | | | | | | | |
| E5 | DNA methylation | 0.4591 | 1.5826 | 80 | 0.1694 | 0.2207 | 0.0067 | 0.4869 | 0.089 |
| FLT3-ITD | SNV/Indel | 0.2481 | 1.2816 | 223 | 0.094 | 0.1554 | 0.0083 | 0.4869 | 0.089 |
| abnormal chr 12 | CNA | 0.2203 | 1.2464 | 40 | 0.0836 | 0.1684 | 0.0084 | 0.4869 | 0.089 |
| (monosomy | | | | | | | | | |
| 12 or del(12p) | | | | | | | | | |
| or abnormal | | | | | | | | | |
| 12p) | | | | | | | | | |
| SHS: WT1 | Gene: DNA methylation | 0.2101 | 1.2338 | 32 | 0.0804 | 0.1935 | 0.0089 | 0.4869 | 0.089 |
| | interaction | | | | | | | | |
| E7: NPMI | Gene: DNA methylation | 0.156 | 1.1689 | 130 | 0.06 | 0.1891 | 0.0093 | 0.4869 | 0.089 |
| | interaction | | | | | | | | |
| SHS: DNMT3A | Gene: DNA methylation | −0.2262 | 0.7975 | 88 | 0.0871 | 0.176 | 0.0094 | 0.4869 | 0.089 |
| | interaction | | | | | | | | |
| monosomy 7 | CNA | 0.223 | 1.2498 | 83 | 0.0875 | 0.1583 | 0.0108 | 0.5261 | 0.0962 |
| monosomy 5 or | CNA | 0.1729 | 1.1887 | 83 | 0.0754 | 0.158 | 0.0218 | 0.9903 | 0.181 |
| del(5g) | | | | | | | | | |
| E8: NPMI | Gene: DNA methylation | −0.1826 | 0.8331 | 156 | 0.0819 | 0.1696 | 0.0258 | 1 | 0.2021 |
| | interaction | | | | | | | | |
| E13 | DNA methylation | 0.1812 | 1.1986 | 109 | 0.0848 | 0.1581 | 0.0327 | 1 | 0.236 |
| monosomy 18 or | CNA | 0.1373 | 1.1472 | 23 | 0.0647 | 0.1833 | 0.0337 | 1 | 0.236 |
| del(18q) | | | | | | | | | |
| TP53 | SNV/Indel | 0.2195 | 1.2454 | 88 | 0.1058 | 0.1816 | 0.038 | 1 | 0.2479 |
| E11: NPM1 | Gene: DNA methylation | −0.1396 | 0.8697 | 36 | 0.0678 | 0.1905 | 0.0394 | 1 | 0.2479 |
| | interaction | | | | | | | | |
| NPM1 | SNV/Indel | −0.175 | 0.8395 | 382 | 0.0856 | 0.1582 | 0.041 | 1 | 0.2479 |
| FLT3-TKD | SNV/Indel | 0.1929 | 1.2128 | 99 | 0.0969 | 0.1664 | 0.0464 | 1 | 0.2683 |
| t(6; 9) | Fusions | 0.2742 | 1.3155 | 6 | 0.1432 | 0.4265 | 0.0554 | 1 | 0.3013 |
| abnormal chr 3q | CNA | 0.1416 | 1.1521 | 12 | 0.0743 | 0.1733 | 0.0566 | 1 | 0.3013 |
| complex karyotype | CNA | 0.1469 | 1.1583 | 121 | 0.078 | 0.1422 | 0.0595 | 1 | 0.3022 |
| abnormal chr 4 | CNA | 0.1296 | 1.1384 | 17 | 0.0693 | 0.1745 | 0.0614 | 1 | 0.3022 |
| (monosomy 4 | | | | | | | | | |
| or del(4p) or | | | | | | | | | |
| abnormal 4p) | | | | | | | | | |
| HB | Clinical | 0.1474 | 1.1588 | 987 | 0.0818 | 0.1643 | 0.0715 | 1 | 0.3395 |
| MLL | SNV/Indel | 0.1528 | 1.1651 | 14 | 0.0868 | 0.2033 | 0.0782 | 1 | 0.3587 |
| del(7q) | CNA | 0.1459 | 1.1571 | 53 | 0.0864 | 0.1578 | 0.0911 | 1 | 0.3972 |
| del(9q) | CNA | 0.1486 | 1.1603 | 15 | 0.0884 | 0.1683 | 0.0926 | 1 | 0.3972 |
| SHS: FLT3 ITD | Gene: DNA methylation | 0.1459 | 1.1571 | 112 | 0.088 | 0.1768 | 0.0974 | 1 | 0.405 |
| | interaction | | | | | | | | |
| TET2 | SNV/Indel | 0.1533 | 1.1657 | 137 | 0.0934 | 0.1543 | 0.1008 | 1 | 0.4057 |
| SF1 | SNV/Indel | 0.1046 | 1.1103 | 9 | 0.0645 | 0.2143 | 0.1049 | 1 | 0.4057 |
| abnormal chr 7 | CNA | 0.1143 | 1.1211 | 12 | 0.0717 | 0.1816 | 0.1108 | 1 | 0.4057 |
| (other) | | | | | | | | | |
| E8 | DNA methylation | 0.1419 | 1.1525 | 224 | 0.0895 | 0.1717 | 0.1129 | 1 | 0.4057 |
| E8: WT1 | Gene: DNA methylation | 0.123 | 1.1309 | 40 | 0.0777 | 0.1876 | 0.1136 | 1 | 0.4057 |
| | interaction | | | | | | | | |
| monosomy 20 or | CNA | 0.1295 | 1.1383 | 28 | 0.0825 | 0.169 | 0.1165 | 1 | 0.4057 |
| del(20q) | | | | | | | | | |
| NPM1: WT1 | Gene: Gene interaction | 0.114 | 1.1207 | 36 | 0.0738 | 0.1917 | 0.1227 | 1 | 0.4057 |
| NPMI: CEBPA- | Gene: Gene interaction | 0.126 | 1.1343 | 27 | 0.0821 | 0.19 | 0.1247 | 1 | 0.4057 |
| mono | | | | | | | | | |
| NPMI: FLT3-TKD | Gene: Gene interaction | −0.1208 | 0.8862 | 60 | 0.0787 | 0.1825 | 0.1251 | 1 | 0.4057 |
| t(v; 11) | Fusions | 0.2942 | 1.342 | 39 | 0.1971 | 0.2473 | 0.1356 | 1 | 0.4293 |
| BCOR | SNV/Indel | 0.1544 | 1.167 | 63 | 0.1049 | 0.1692 | 0.1411 | 1 | 0.436 |
| DNMT3A: PTPN11 | Gene: Gene interaction | −0.1188 | 0.888 | 34 | 0.0816 | 0.1874 | 0.1452 | 1 | 0.436 |
| t(9; 11) | Fusions | 0.3154 | 1.3708 | 24 | 0.2178 | 0.3113 | 0.1475 | 1 | 0.436 |
| minusY | CNA | 0.1201 | 1.1277 | 35 | 0.0847 | 0.1679 | 0.1563 | 1 | 0.4455 |
| PB Blasts | Clinical | 0.0319 | 1.0324 | 1015 | 0.0226 | 0.0241 | 0.1574 | 1 | 0.4455 |
| E3 | DNA methylation | −0.2429 | 0.7843 | 26 | 0.18 | 0.3102 | 0.1771 | 1 | 0.4907 |
| plus13 | CNA | 0.1043 | 1.11 | 23 | 0.0787 | 0.1778 | 0.1847 | 1 | 0.5014 |
| LDH | Clinical | 0.0744 | 1.0772 | 759 | 0.0575 | 0.1811 | 0.1959 | 1 | 0.521 |
| E9 | DNA methylation | 0.1106 | 1.1169 | 22 | 0.0868 | 0.1985 | 0.2028 | 1 | 0.5289 |
| NPM1: FLT3-ITD | Gene: Gene interaction | 0.1051 | 1.1109 | 136 | 0.0848 | 0.1718 | 0.2151 | 1 | 0.5447 |
| U2AF1 | SNV/Indel | 0.1197 | 1.1271 | 40 | 0.097 | 0.189 | 0.2171 | 1 | 0.5447 |
| E8: TET2 | Gene: DNA methylation | 0.1017 | 1.107 | 34 | 0.0833 | 0.1922 | 0.2224 | 1 | 0.5467 |
| | interaction | | | | | | | | |
| NPM1: NRAS | Gene: Gene interaction | 0.1035 | 1.109 | 67 | 0.0855 | 0.1775 | 0.2261 | 1 | 0.5467 |
| NPM1: IDH2 p140 | Gene: Gene interaction | −0.1 | 0.9049 | 47 | 0.0845 | 0.179 | 0.237 | 1 | 0.5628 |
| plus21 | CNA | 0.1006 | 1.1058 | 25 | 0.0869 | 0.1683 | 0.247 | 1 | 0.5675 |
| WTI: FLT3-ITD | Gene: Gene interaction | 0.0937 | 1.0983 | 31 | 0.081 | 0.1904 | 0.2475 | 1 | 0.5675 |
| ETV6 | SNV/Indel | 0.1078 | 1.1138 | 25 | 0.0996 | 0.1873 | 0.2794 | 1 | 0.6298 |
| E7: FLT3_ITD | Gene: DNA methylation | 0.0789 | 1.0821 | 56 | 0.0753 | 0.189 | 0.2952 | 1 | 0.6544 |
| | interaction | | | | | | | | |
| CEBPA-dm | SNV/Indel | 0.0994 | 1.1045 | 72 | 0.0977 | 0.1808 | 0.309 | 1 | 0.6737 |
| plus8 or +8q | CNA | 0.0879 | 1.0919 | 104 | 0.091 | 0.1448 | 0.3342 | 1 | 0.7036 |
| CEBPA-sm | SNV/Indel | −0.0982 | 0.9065 | 54 | 0.1017 | 0.1701 | 0.3344 | 1 | 0.7036 |
| DNMT3A: IDH2 | Gene: Gene interaction | 0.0795 | 1.0827 | 33 | 0.083 | 0.1915 | 0.3386 | 1 | 0.7036 |
| p140 | | | | | | | | | |
| IDH2 p172 | SNV/Indel | −0.0878 | 0.916 | 33 | 0.0993 | 0.1886 | 0.377 | 1 | 0.7444 |
| JAK2 | SNV/Indel | 0.062 | 1.064 | 5 | 0.0702 | 0.212 | 0.3772 | 1 | 0.7444 |
| PHF6 | SNV/Indel | 0.09 | 1.0942 | 27 | 0.102 | 0.1845 | 0.3774 | 1 | 0.7444 |
| NPMI: TET2 | Gene: Gene interaction | −0.075 | 0.9277 | 64 | 0.0869 | 0.176 | 0.3883 | 1 | 0.7444 |
| WT1 | SNV/Indel | 0.0839 | 1.0875 | 86 | 0.0994 | 0.1625 | 0.3985 | 1 | 0.7444 |
| Splenomegaly | Clinical | 0.026 | 1.0263 | 69 | 0.0313 | 0.1892 | 0.4063 | 1 | 0.7444 |
| tAML | Clinical | 0.026 | 1.0263 | 7 | 0.0313 | 0.1892 | 0.4063 | 1 | 0.7444 |
| SF3A1 | SNV/Indel | 0.0604 | 1.0622 | 9 | 0.0729 | 0.2112 | 0.4079 | 1 | 0.7444 |
| plus22 | CNA | 0.0668 | 1.0691 | 25 | 0.0818 | 0.1693 | 0.4141 | 1 | 0.7444 |
| SHS | DNA methylation | 0.0766 | 1.0796 | 213 | 0.0941 | 0.1567 | 0.4156 | 1 | 0.7444 |
| IKZF1 | SNV/Indel | 0.0776 | 1.0807 | 20 | 0.0961 | 0.1957 | 0.4198 | 1 | 0.7444 |
| t(8; 21) | Fusions | 0.1558 | 1.1686 | 22 | 0.1965 | 0.3416 | 0.4278 | 1 | 0.7487 |
| DNMT3A: IDHI | Gene: Gene interaction | 0.0633 | 1.0653 | 42 | 0.0848 | 0.1828 | 0.4554 | 1 | 0.7776 |
| E8: FLT3_TKD | Gene: DNA methylation | −0.0618 | 0.9401 | 36 | 0.0835 | 0.1862 | 0.4597 | 1 | 0.7776 |
| | interaction | | | | | | | | |
| RAD21 | SNV/Indel | 0.073 | 1.0757 | 24 | 0.0998 | 0.189 | 0.4648 | 1 | 0.7776 |
| BM Blasts | Clinical | −0.0216 | 0.9786 | 1014 | 0.0301 | 0.0321 | 0.4721 | 1 | 0.7776 |
| DNMT3A: NRAS | Gene: Gene interaction | 0.0622 | 1.0642 | 47 | 0.0879 | 0.1831 | 0.479 | 1 | 0.7776 |
| Platelets | Clinical | −0.0499 | 0.9513 | 1002 | 0.0706 | 0.0805 | 0.4795 | 1 | 0.7776 |
| TET2: FLT3-ITD | Gene: Gene interaction | 0.0607 | 1.0626 | 34 | 0.087 | 0.1878 | 0.4853 | 1 | 0.7776 |
| E8: NRAS | Gene: DNA methylation | −0.0542 | 0.9472 | 39 | 0.0864 | 0.1835 | 0.5302 | 1 | 0.826 |
| | interaction | | | | | | | | |
| E4: GATA2 | Gene: DNA methylation | −0.0443 | 0.9567 | 27 | 0.0721 | 0.1962 | 0.5387 | 1 | 0.826 |
| | interaction | | | | | | | | |
| ECOG Performance | Clinical | −0.0394 | 0.9613 | 880 | 0.0648 | 0.0726 | 0.5425 | 1 | 0.826 |
| status | | | | | | | | | |
| E2 | DNA methylation | −0.1224 | 0.8848 | 29 | 0.2029 | 0.3041 | 0.5464 | 1 | 0.826 |
| ZRSR2 | SNV/Indel | 0.0643 | 1.0664 | 49 | 0.1066 | 0.1626 | 0.5465 | 1 | 0.826 |
| NPMI: FLT3- | Gene: Gene interaction | 0.0431 | 1.044 | 65 | 0.0733 | 0.1869 | 0.557 | 1 | 0.8272 |
| ITD: DNMT3A | | | | | | | | | |
| EZH2 | SNV/Indel | 0.0586 | 1.0603 | 27 | 0.1035 | 0.1842 | 0.5713 | 1 | 0.8272 |
| DNMT3A: TET2 | Gene: Gene interaction | 0.0489 | 1.0502 | 31 | 0.0865 | 0.1853 | 0.5715 | 1 | 0.8272 |
| SRFS2 | SNV/Indel | 0.0552 | 1.0568 | 74 | 0.0987 | 0.178 | 0.5759 | 1 | 0.8272 |
| KRAS | SNV/Indel | −0.0585 | 0.9432 | 55 | 0.11 | 0.1679 | 0.5948 | 1 | 0.8272 |
| SHS: E7 | DNA methylation: DNA | 0.0412 | 1.0421 | 65 | 0.0778 | 0.1871 | 0.5961 | 1 | 0.8272 |
| | methylation | | | | | | | | |
| NRAS | SNV/Indel | 0.0488 | 1.05 | 167 | 0.0971 | 0.1448 | 0.6155 | 1 | 0.8272 |
| BRAF | SNV/Indel | 0.0337 | 1.0343 | 6 | 0.0675 | 0.2093 | 0.6172 | 1 | 0.8272 |
| E4: CEBPA_bi | Genc: DNA methylation | −0.0327 | 0.9678 | 57 | 0.0664 | 0.1958 | 0.622 | 1 | 0.8272 |
| | interaction | | | | | | | | |
| DNMT3A: NPM1 | Gene: Gene interaction | 0.0398 | 1.0406 | 182 | 0.0808 | 0.1716 | 0.6225 | 1 | 0.8272 |
| E7: DNMT3A | Gene: DNA methylation | 0.0295 | 1.0299 | 119 | 0.0621 | 0.1889 | 0.6349 | 1 | 0.8272 |
| | interaction | | | | | | | | |
| E8: PTPN11 | Gene: DNA methylation | −0.0394 | 0.9614 | 39 | 0.083 | 0.1897 | 0.6355 | 1 | 0.8272 |
| | interaction | | | | | | | | |
| WBC | Clinical | 0.0391 | 1.0399 | 1007 | 0.0831 | 0.1027 | 0.6379 | 1 | 0.8272 |
| SRFS2: IDH2_p140 | Gene: Gene interaction | 0.036 | 1.0366 | 29 | 0.0769 | 0.1926 | 0.64 | 1 | 0.8272 |
| GATA2: CEBPA-bi | Gene: Gene interaction | 0.0339 | 1.0345 | 27 | 0.0735 | 0.1947 | 0.6445 | 1 | 0.8272 |
| PTEN | SNV/Indel | 0.0307 | 1.0311 | 5 | 0.0672 | 0.2132 | 0.6481 | 1 | 0.8272 |
| E10 | DNA methylation | −0.0359 | 0.9647 | 40 | 0.0799 | 0.1951 | 0.653 | 1 | 0.8272 |
| STAG2 | SNV/Indel | 0.0423 | 1.0432 | 30 | 0.0977 | 0.1805 | 0.6649 | 1 | 0.8279 |
| IDH2 p140 | SNV/Indel | 0.0384 | 1.0391 | 91 | 0.0899 | 0.1648 | 0.6698 | 1 | 0.8279 |
| ASXL1: RUNX1 | Gene: Gene interaction | −0.0325 | 0.968 | 25 | 0.0769 | 0.1936 | 0.6723 | 1 | 0.8279 |
| NF1 | SNV/Indel | 0.0394 | 1.0401 | 47 | 0.1003 | 0.1817 | 0.6948 | 1 | 0.8398 |
| E11 | DNA methylation | −0.0379 | 0.9628 | 55 | 0.0973 | 0.1713 | 0.6968 | 1 | 0.8398 |
| DNMT3A: FLT3- | Gene: Gene interaction | 0.0305 | 1.031 | 85 | 0.0795 | 0.1811 | 0.7009 | 1 | 0.8398 |
| ITD | | | | | | | | | |
| ASXL1 | SNV/Indel | 0.0372 | 1.0379 | 68 | 0.0996 | 0.1729 | 0.7089 | 1 | 0.8418 |
| RUNX1 | SNV/Indel | 0.0364 | 1.0371 | 112 | 0.1007 | 0.1486 | 0.7179 | 1 | 0.8427 |
| DNMT3A: FLT3- | Gene: Gene interaction | 0.0278 | 1.0282 | 31 | 0.0793 | 0.1942 | 0.7257 | 1 | 0.8427 |
| TKD | | | | | | | | | |
| NPM1: PTPN11 | Gene: Gene interaction | −0.0253 | 0.975 | 64 | 0.073 | 0.1837 | 0.7287 | 1 | 0.8427 |
| E12 | DNA methylation | 0.0271 | 1.0275 | 180 | 0.0832 | 0.1443 | 0.7446 | 1 | 0.8538 |
| inv(16) | Fusions | −0.0532 | 0.9482 | 24 | 0.1798 | 0.3263 | 0.7671 | 1 | 0.872 |
| ASXL1: SRFS2 | Gene: Gene interaction | 0.0195 | 1.0197 | 25 | 0.0733 | 0.1991 | 0.7904 | 1 | 0.887 |
| MPL | SNVAndel | 0.0238 | 1.0241 | 11 | 0.0911 | 0.1958 | 0.7936 | 1 | 0.887 |
| IDH1: NPM1 | Gene: Gene interaction | 0.0176 | 1.0177 | 49 | 0.0833 | 0.1819 | 0.833 | 1 | 0.9172 |
| KIT | SNV/Indel | −0.0214 | 0.9788 | 40 | 0.1025 | 0.1784 | 0.8344 | 1 | 0.9172 |
| SHS: NPM1 | Gene: DNA methylation | −0.016 | 0.9842 | 117 | 0.0864 | 0.1711 | 0.8534 | 1 | 0.9303 |
| | interaction | | | | | | | | |
| NF1: NPM1 | Gene: Gene interaction | 0.0131 | 1.0132 | 18 | 0.0745 | 0.1986 | 0.8606 | 1 | 0.9305 |
| RUNX1: SRFS2 | Gene: Gene interaction | 0.0126 | 1.0127 | 25 | 0.0769 | 0.197 | 0.8697 | 1 | 0.9328 |
| GATA2 | SNV/Indel | −0.0141 | 0.986 | 57 | 0.1022 | 0.1741 | 0.89 | 1 | 0.9442 |
| E8: DNMT3A | Gene: DNA methylation | −0.0111 | 0.989 | 45 | 0.087 | 0.1799 | 0.8984 | 1 | 0.9442 |
| | interaction | | | | | | | | |
| SF3B1 | SNV/Indel | −0.0133 | 0.9868 | 37 | 0.1078 | 0.1784 | 0.902 | 1 | 0.9442 |
| DNMT3A | SNV/Indel | 0.0104 | 1.0104 | 266 | 0.0904 | 0.1532 | 0.9087 | 1 | 0.9442 |
| E4 | DNA methylation | −0.017 | 0.9831 | 74 | 0.1912 | 0.2835 | 0.929 | 1 | 0.946 |
| PTPN11 | SNV/Indel | −0.0078 | 0.9922 | 101 | 0.0902 | 0.1725 | 0.9309 | } | 0.946 |
| SHS: E8 | DNA methylation: DNA | 0.0072 | 1.0072 | 71 | 0.0844 | 0.1766 | 0.9318 | 1 | 0.946 |
| | methylation | | | | | | | | |
| NRAS: TET2 | Gene: Gene interaction | −0.0038 | 0.9962 | 25 | 0.0891 | 0.1839 | 0.9662 | 1 | 0.9736 |
| Gender | Demographics | −0.0011 | 0.9989 | 1021 | 0.102 | 0.1121 | 0.991 | 1 | 0.991 |

TABLE 12
Adjustment of raw methylation-iPLEX values to correspond with Illumina probe beta values

| | | | | Adjusted | |
|---|---|---|---|---|---|
| | | | | Coefficient of | R² |
| | | | | Determination | (independent |
| Assay Name | Gene Symbol | Illumina ID | Polynomial Regression Equation | (R²)† | validation)† † |
| TULP4 | TULP4 | cg00393348 | y = 0.05809 + −0.6427x + 4.430x\hat{ }2 + −2.914x\hat{ }3 | 0.972 | 0.898 |
| ZNF438 | ZNF438 | cg00428179 | y = 0.01026 + 0.1385x + 1.455x\hat{ }2 + −0.6646x\hat{ }3 | 0.921 | 0.667 |
| TM4SF19 | TM4SF19 | cg01883662 | y = 0.1979 + −1.323x + 4.329x\hat{ }2 + −2.260x\hat{ }3 | 0.921 | 0.625 |
| RGS12 | RGS12 | cg01919885 | y = 0.05813 + 0.7414x + 2.347x\hat{ }2 + −2.338x\hat{ }3 | 0.973 | 0.868 |
| HOXB3.1 | HOXB3 | cg01990102 | y = 0.03957 + 0.2985x + 1.858x\hat{ }2 + −1.305x\hat{ }3 | 0.986 | 0.924 |
| WT1 | WT1 | cg03052301 | y = (−0.0009366) + 0.5325x + 0.3240x\hat{ }2 + | 0.975 | 0.905 |
| | | | 0.2743x\hat{ }3 | | |
| CD34.1 | CD34 | cg03583857 | y = 0.1158 + 0.2291x + 2.320x\hat{ }2 + −1.715x\hat{ }3 | 0.964 | 0.929 |
| HIVEP3 | HIVEP3 | cg03884592 | y = 0.07705 + −1.179x + 5.075x\hat{ }2 + −3.098x\hat{ }3 | 0.966 | 0.878 |
| HOXB3.2.2 | HOXB3 | cg04117801 | y = 0.03886 + 1.304x+ −0.4394x\hat{ }2 + 0.1166x\hat{ }3 | 0.959 | 0.671 |
| LRPAP1 | LRPAP1 | cg04857395 | y = 0.06171 + −1.581x + 6.902x\hat{ }2 + −4.454x\hat{ }3 | 0.963 | 0.937 |
| PDYN-AS1 | PDYN-AS1 | cg07210840 | y = 0.05797 + 0.03776x + 2.682x\hat{ }2 + −1.869x\hat{ }3 | 0.968 | 0.919 |
| ZSCAN25 | ZSCAN25 | cg07375256 | y = 0.02788 + 0.1801x + 2.011x\hat{ }2 + −1.237x\hat{ }3 | 0.990 | 0.956 |
| HOXB-AS3.1 | HOXB-AS3 | cg07676709 | y = 0.07931 + −0.1544x + 2.471x\hat{ }2 + −1.510x\hat{ }3 | 0.977 | 0.871 |
| PALM.2 | PALM | cg07876162 | y = 0.1223 + 1.151x + 1.442x\hat{ }2 + −2.143x\hat{ }3 | 0.899 | 0.856 |
| ZZEF1 | ZZEF1 | cg08166720 | y = 0.05021 + −0.2306x + 1.806x\hat{ }2 + −0.6419x\hat{ }3 | 0.975 | 0.531 |
| GIMAP7 | GIMAP7 | cg08637514 | y = 0.01372 + −0.07764x + 3.146x\hat{ }2 + −2.106x\hat{ }3 | 0.987 | 0.949 |
| ESRP2 | ESRP2 | cg08694699 | y = 0.09514 + 0.5532x + 0.4061x\hat{ }2 + −0.1951x\hat{ }3 | 0.955 | 0.706 |
| CTTN | CTTN | cg09352338 | y = 0.1641 + -2.071x + 8.240x\hat{ }2 + −5.515x\hat{ }3 | 0.947 | 0.857 |
| DNMT3A.1 | DNMT3A | cg10239163 | y = 0.07693 + 1.178x + −0.6478x\hat{ }2 + 0.3036x\hat{ }3 | 0.963 | 0.885 |
| CELF2 | CELF2 | cg11002119 | y = 0.1109 + −0.6277x + 4.557x\hat{ }2 + −3.081x\hat{ }3 | 0.982 | 0.932 |
| MED13L | MED13L | cg12220034 | y = 0.3411 + −0.6795x + 2.799x\hat{ }2 + −1.492x\hat{ }3 | 0.911 | 0.840 |
| MLLT10 | MLLT10 | cg12225526 | y = 0.07036 + 0.1055x + 0.2936x\hat{ }2 + 0.4686x\hat{ }3 | 0.946 | 0.869 |
| MEF2B | MEF2B | cg12558012 | y = 0.01513 + 0.9348x + 2.346x\hat{ }2 + −2.481x\hat{ }3 | 0.980 | 0.912 |
| CCDC9B | CCDC9B | cg12732548 | y = 0.04310 + 0.5650x + 1.972x\hat{ }2 + −1.652x\hat{ }3 | 0.972 | 0.840 |
| HOXB3.2.1 | HOXB3 | cg13293524 | y = 0.05511 + 0.1442x + 2.098x\hat{ }2 + −1.385x\hat{ }3 | 0.932 | 0.657 |
| A4GALT | A4GALT | cg15429214 | y = 0.1304 + 0.04642x + 2.190x\hat{ }2 + −1.461x\hat{ }3 | 0.969 | 0.943 |
| CHML | CHML | cg15775914 | y = 0.05081 + 0.1627x + 1.955x\hat{ }2 + −1.149x\hat{ }3 | 0.973 | 0.862 |
| ACOT7 | ACOT7 | cg16034168 | y = 0.1235 + −1.635x + 5.844x\hat{ }2 + −3.366x\hat{ }3 | 0.924 | 0.826 |
| PRKAG2 | PRKAG2 | cg17192599 | y = 0.1280 + −2.106x + 7.617x\hat{ }2 + −4.693x\hat{ }3 | 0.977 | 0.854 |
| AIM2 | AIM2 | cg17515347 | y = 0.05808 + 0.2450x + 1.418x\hat{ }2 + −0.8441x\hat{ }3 | 0.948 | 0.897 |
| MIRLET7BHG | MIRLET7BHG | cg18066206 | y = 0.04883 + −0.4176x + 2.771x\hat{ }2 + −1.490x\hat{ }3 | 0.980 | 0.864 |
| REC8 | REC8 | cg18628371 | y = 0.04586 + −0.3698x + 2.615x\hat{ }2 + −1.205x\hat{ }3 | 0.957 | 0.961 |
| BEND7 | BEND7 | cg19695507 | y = 0.03068 + 1.800x + −2.419x\hat{ }2 + 1.218x\hat{ }3 | 0.876 | 0.967 |
| HMGA1 | HMGA1 | cg20294304 | y = 0.02942 + 1.134x + 0.05196x\hat{ }2 + −0.3010x\hat{ }3 | 0.964 | 0.899 |
| HCCA2 | HCCA2 | cg20299572 | y = 0.03737 + 0.2790x + 0.4673x\hat{ }2 + 0.05377x\hat{ }3 | 0.984 | 0.929 |
| HOXB-AS3.2 | HOXB-AS3 | cg21816532 | y = 0.08912 + 1.660x + −1.341x\hat{ }2 + 0.4578x\hat{ }3 | 0.896 | 0.830 |
| XXYLT1 | XXYLT1 | cg21937377 | y = 0.1015 + −1.761x + 10.74x\hat{ }2 + −9.327x\hat{ }3 | 0.956 | 0.860 |
| DNMT3A.2 | DNMT3A | cg23903708 | y = 0.07197 + 0.7044x + 1.566x\hat{ }2 + −1.399x\hat{ }3 | 0.981 | 0.855 |
| HOXB3.3 | HOXB3 | cg24767968 | y = 0.2458 + −2.448x + 6.923x\hat{ }2 + −3.880x\hat{ }3 | 0.952 | 0.924 |
| ALS2CL | ALS2CL | cg25104512 | y = 0.02332 + 0.1960x + 1.132x\hat{ }2 + −0.4252x\hat{ }3 | 0.951 | 0.876 |
| PPPIR18 | PPPIR18 | cg25659902 | y = 0.02726 + −0.5228x + 3.845x\hat{ }2 + −2.359x\hat{ }3 | 0.986 | 0.625 |
| CD34.2 | CD34 | cg26266618 | y = 0.04944 + −1.366x + 6.568x\hat{ }2 + −4.393x\hat{ }3 | 0.982 | 0.938 |
| PALM.1 | PALM | cg27183173 | y = 0.09841 + −0.5436x + 4.729x\hat{ }2 + −3.421x\hat{ }3 | 0.936 | 0.830 |

†Calculated using comparison of n = 223 samples from the Beat AML cohort
† †Calculated using comparison of n = 139 samples from an independent cohort

TABLE 13
Annotation CpGs composing the STAT Hypomethylation
Signature (SHS) Me-iPLEX assay

| | | | | Position |
|---|---|---|---|---|
| | | | Position | relative to |
| Assay Name | Illumina ID | Chromosome | (hg19) | gene |
| PRDM16 | cg17104202 | 1 | 3162404 | Body |
| CA6 | cg25919221 | 1 | 9006680 | Body |
| EDN2 | cg16736826 | 1 | 41951512 | TSS1500 |
| HS2ST1 | cg17907457 | 1 | 87416597 | Body |
| LRRC8D | cg03899643 | 1 | 90205170 | Intergenic |
| ST3GAL5 | cg04849850 | 2 | 86121000 | 5′Region |
| NOSTRIN | cg00174992 | 2 | 169659121 | 5′UTR |
| CISH | cg08996521 | 3 | 50649994 | TSS1500 |
| ARHGEF3 | cg18482892 | 3 | 56833426 | Body |
| VGLL3 | cg15867626 | 3 | 87045569 | 5′Region |
| CD80 | cg13458803 | 3 | 119276917 | 5′UTR |
| ADPRH | cg10994564 | 3 | 119306022 | Body |
| MUC4 | cg18513344 | 3 | 195531298 | Body |
| TRIM15 | cg00720829 | 6 | 30131219 | 5′UTR |
| CDKN1A | cg17526952 | 6 | 36643854 | TSS1500 |
| HEATR2 | cg10472711 | 7 | 797592 | Body |
| DLC1 | cg10941185 | 8 | 12988516 | Body |
| GPR124 | cg18715243 | 8 | 37658755 | Body |
| RAB11FIP1 | cg17218270 | 8 | 37749412 | Body |
| FAS | cg23195687 | 10 | 90847168 | Intergenic |
| DUSP5 | cg10080966 | 10 | 112260867 | Body |
| SMC3 | cg01269299 | 10 | 112287792 | Intergenic |
| CRADD | cg21359950 | 12 | 94083470 | Body |
| STARD13 | cg16522412 | 13 | 33926811 | 5′UTR |
| ACSBG1 | cg17519101 | 15 | 78526804 | 1stExon |
| TBC1D16 | cg00973876 | 17 | 77899208 | Intergenic |
| MAPRE2 | cg25477456 | 18 | 32552858 | 5′Region |
| SBNO2 | cg07573872 | 19 | 1126342 | Body |
| TXN2 | cg16549994 | 22 | 36854045 | Intergenic |

TABLE 14
Alliance Average Probe Heatmap Raw Data

| Probe name | E1 | E2 | E3 | E4 | E5 | E6 |
|---|---|---|---|---|---|---|
| ZSCAN25 | 9.6% | 21.9% | 14.6% | 4.7% | 41.4% | 17.1% |
| ESRP2 | 46.9% | 28.9% | 25.8% | 80.8% | 13.2% | 57.1% |
| RGS12 | 10.8% | 11.6% | 11.2% | 12.2% | 17.5% | 21.8% |
| HIVEP3 | 79.5% | 62.5% | 72.9% | 82.4% | 32.5% | 64.3% |
| ALS2CL | 43.7% | 9.7% | 10.5% | 28.1% | 9.6% | 14.9% |
| PDYN-AS1 | 87.3% | 57.3% | 20.7% | 77.5% | 9.8% | 58.6% |
| HCCA2 | 10.9% | 14.0% | 76.2% | 6.7% | 14.6% | 10.4% |
| HOXB3.3 | 86.5% | 90.8% | 98.7% | 84.4% | 46.2% | 84.0% |
| PALM.2 | 98.0% | 32.6% | 20.6% | 72.5% | 48.7% | 71.8% |
| CELF2 | 90.9% | 96.0% | 92.8% | 97.0% | 25.2% | 23.8% |
| MED13L | 100.0% | 97.9% | 96.9% | 99.9% | 36.5% | 89.0% |
| CTTN | 63.9% | 95.3% | 20.2% | 87.8% | 47.7% | 34.3% |
| PRKAG2 | 19.5% | 27.4% | 82.7% | 98.9% | 42.9% | 61.5% |
| MLLT10 | 41.7% | 93.3% | 90.0% | 92.7% | 57.8% | 60.8% |
| BEND7 | 1.3% | 2.9% | 3.9% | 1.5% | 5.5% | 2.5% |
| ZNF438 | 98.4% | 87.7% | 84.4% | 96.4% | 51.3% | 91.9% |
| HOXB-AS3.2 | 7.7% | 8.1% | 7.7% | 7.9% | 8.6% | 8.9% |
| CD34.2 | 99.6% | 6.4% | 5.4% | 22.0% | 35.6% | 61.0% |
| TM4SF19 | 45.6% | 67.2% | 31.2% | 82.5% | 60.5% | 78.8% |
| ZZEF1 | 100.0% | 17.7% | 93.9% | 98.3% | 95.0% | 99.3% |
| ACOT7 | 100.0% | 27.7% | 95.7% | 87.7% | 94.3% | 98.6% |
| HOXB-AS3.1 | 92.8% | 76.6% | 77.7% | 79.0% | 56.1% | 69.4% |
| MIRLET7BHG | 97.7% | 96.2% | 95.8% | 93.7% | 33.0% | 68.4% |
| MEF2B | 20.7% | 9.7% | 11.5% | 68.3% | 6.4% | 27.8% |
| DNMT3A.1 | 20.0% | 75.6% | 47.1% | 88.2% | 34.9% | 51.2% |
| PPP1R18 | 66.6% | 1.5% | 1.9% | 2.9% | 2.8% | 5.0% |
| REC8 | 78.7% | 70.1% | 28.9% | 74.3% | 21.0% | 40.2% |
| PALM.1 | 96.8% | 15.6% | 13.2% | 57.4% | 32.8% | 62.5% |
| DNMT3A.2 | 32.2% | 82.6% | 61.8% | 95.4% | 51.1% | 69.1% |
| WT1 | 86.5% | 33.1% | 78.3% | 29.1% | 10.9% | 64.5% |
| GIMAP7 | 56.5% | 85.3% | 43.8% | 77.6% | 26.8% | 69.9% |
| TULP4 | 96.5% | 69.9% | 92.9% | 20.0% | 73.9% | 90.0% |
| XXYLT1 | 82.7% | 64.2% | 60.6% | 66.2% | 30.5% | 63.9% |
| CCDC9B | 88.1% | 70.1% | 22.4% | 87.7% | 74.6% | 93.2% |
| AIM2 | 88.4% | 47.2% | 55.6% | 57.5% | 51.1% | 51.6% |
| A4GALT | 25.3% | 23.3% | 26.7% | 71.3% | 31.4% | 38.7% |
| CHML | 90.5% | 88.3% | 84.0% | 85.8% | 31.5% | 73.1% |
| CD34.1 | 99.9% | 18.8% | 23.0% | 21.3% | 50.5% | 72.7% |
| HOXB3.2.2 | 100.0% | 96.3% | 99.6% | 93.9% | 69.2% | 92.6% |
| LRPAP1 | 5.9% | 31.3% | 37.9% | 41.2% | 95.4% | 96.5% |
| HOXB3.1 | 5.0% | 15.2% | 85.8% | 15.2% | 10.6% | 37.9% |
| HMGA1 | 14.4% | 5.5% | 2.2% | 11.3% | 10.1% | 12.6% |
| HOXB3.2.1 | 60.2% | 90.1% | 95.7% | 89.5% | 59.5% | 87.0% |

TABLE 15
Training Average Probe Heatmap Raw Data

| Probe name | E1 | E2 | E3 | E4 | E5 | E6 |
|---|---|---|---|---|---|---|
| ZSCAN25 | 9.9% | 15.3% | 14.5% | 6.1% | 41.5% | 13.3% |
| HCCA2 | 12.0% | 10.7% | 73.5% | 6.2% | 17.5% | 8.3% |
| RGS12 | 11.9% | 9.9% | 7.3% | 12.0% | 17.6% | 27.0% |
| HOXB3.1 | 16.1% | 14.3% | 79.7% | 27.7% | 14.5% | 89.4% |
| BEND7 | 16.9% | 4.7% | 4.4% | 4.0% | 5.0% | 6.1% |
| ALS2CL | 3.8% | 3.5% | 3.2% | 25.3% | 2.9% | 13.7% |
| HMGA1 | 48.7% | 5.2% | 5.3% | 25.4% | 10.9% | 25.9% |
| HOXB-AS3.2 | 6.3% | 5.7% | 5.8% | 6.4% | 8.4% | 55.7% |
| PPPIR18 | 77.3% | 1.3% | 1.7% | 1.8% | 1.4% | 3.0% |
| DNMT3A.2 | 27.1% | 85.7% | 69.8% | 95.6% | 62.2% | 89.8% |
| MLLT10 | 52.1% | 90.9% | 93.9% | 92.7% | 68.3% | 90.0% |
| DNMT3A.1 | 18.7% | 74.1% | 54.7% | 89.6% | 42.9% | 75.3% |
| PRKAG2 | 15.8% | 32.5% | 84.9% | 87.5% | 59.4% | 92.0% |
| TM4SF19 | 29.9% | 59.8% | 15.4% | 91.0% | 65.3% | 91.3% |
| CCDC9B | 54.9% | 62.2% | 14.3% | 84.0% | 74.6% | 86.6% |
| ZNF438 | 93.5% | 91.6% | 88.7% | 94.2% | 52.2% | 93.1% |
| MED13L | 96.6% | 96.5% | 92.9% | 96.0% | 63.8% | 95.6% |
| CHML | 91.2% | 87.2% | 83.4% | 83.9% | 43.8% | 84.3% |
| TULP4 | 95.2% | 58.6% | 90.1% | 23.4% | 79.0% | 91.2% |
| ZZEF1 | 98.4% | 14.7% | 96.3% | 98.0% | 94.9% | 91.9% |
| ACOT7 | 95.8% | 19.6% | 94.6% | 86.5% | 94.6% | 95.4% |
| LRPAP1 | 17.1% | 10.8% | 11.1% | 22.2% | 84.0% | 95.6% |
| PALM.2 | 63.3% | 26.9% | 14.1% | 72.8% | 42.7% | 82.7% |
| PALM.1 | 62.1% | 29.1% | 16.7% | 75.3% | 45.0% | 81.1% |
| ESRP2 | 43.9% | 21.2% | 24.2% | 78.0% | 20.8% | 86.2% |
| MEF2B | 15.0% | 11.1% | 7.0% | 74.9% | 4.8% | 74.0% |
| REC8 | 67.9% | 64.0% | 14.9% | 67.2% | 12.8% | 78.9% |
| PDYN-AS1 | 37.4% | 59.3% | 15.5% | 74.1% | 19.3% | 83.2% |
| GIMAP7 | 69.6% | 74.0% | 43.2% | 71.5% | 29.8% | 85.9% |
| XXYLT1 | 87.0% | 79.3% | 77.8% | 84.6% | 36.4% | 86.1% |
| HIVEP3 | 52.6% | 56.1% | 81.8% | 77.2% | 22.3% | 83.7% |
| WT1 | 75.3% | 27.4% | 71.0% | 27.5% | 16.3% | 84.4% |
| CD34.2 | 85.0% | 12.4% | 8.8% | 14.0% | 29.0% | 27.7% |
| CD34.1 | 90.2% | 26.4% | 21.1% | 20.8% | 42.1% | 41.3% |
| AIM2 | 20.8% | 86.3% | 87.5% | 85.2% | 31.8% | 35.5% |
| A4GALT | 34.4% | 20.7% | 20.7% | 75.3% | 39.0% | 24.5% |
| CTTN | 36.0% | 83.3% | 19.8% | 77.7% | 47.5% | 25.6% |
| CELF2 | 81.8% | 95.6% | 93.5% | 95.7% | 34.6% | 67.5% |
| HOXB-AS3.1 | 76.5% | 78.1% | 80.6% | 83.9% | 47.4% | 38.3% |
| MIRLET7BHG | 93.4% | 92.8% | 92.7% | 92.6% | 33.6% | 89.8% |
| HOXB3.2.2 | 86.7% | 87.6% | 90.5% | 88.3% | 63.5% | 91.3% |
| HOXB3.2.1 | 91.7% | 95.2% | 94.9% | 93.9% | 73.6% | 95.0% |
| HOXB3.3 | 81.9% | 72.2% | 85.8% | 78.0% | 51.3% | 89.2% |

| Probe name | E7 | E8 | E9 | E10 | E11 | E12 | E13 |
|---|---|---|---|---|---|---|---|
| ZSCAN25 | 9.0% | 8.5% | 66.7% | 81.0% | 85.3% | 73.8% | 67.8% |
| HCCA2 | 6.5% | 8.4% | 56.3% | 81.0% | 79.1% | 46.1% | 58.2% |
| RGS12 | 11.8% | 23.7% | 9.2% | 54.1% | 88.5% | 72.3% | 69.2% |
| HOXB3.1 | 6.4% | 11.8% | 12.1% | 55.4% | 69.5% | 59.0% | 31.5% |
| BEND7 | 3.7% | 4.6% | 23.2% | 29.2% | 64.7% | 6.6% | 24.4% |
| ALS2CL | 2.9% | 4.9% | 9.0% | 66.7% | 35.5% | 14.1% | 8.6% |
| HMGA1 | 11.1% | 18.1% | 46.4% | 81.2% | 73.9% | 20.0% | 22.0% |
| HOXB-AS3.2 | 17.1% | 68.8% | 40.8% | 61.8% | 18.7% | 16.3% | 9.0% |
| PPPIR18 | 1.4% | 2.6% | 17.8% | 13.3% | 6.4% | 1.5% | 2.3% |
| DNMT3A.2 | 83.6% | 91.7% | 92.4% | 95.8% | 96.3% | 92.1% | 93.7% |
| MLLT10 | 68.6% | 86.9% | 93.1% | 93.2% | 91.7% | 90.7% | 90.7% |
| DNMT3A.1 | 62.8% | 75.5% | 89.9% | 93.9% | 92.5% | 81.2% | 82.1% |
| PRKAG2 | 91.4% | 95.5% | 96.4% | 95.9% | 94.3% | 92.8% | 92.0% |
| TM4SF19 | 78.6% | 86.8% | 90.8% | 94.4% | 92.0% | 86.4% | 88.9% |
| CCDC9B | 83.5% | 86.9% | 88.5% | 88.1% | 80.7% | 87.7% | 79.3% |
| ZNF438 | 90.2% | 94.2% | 87.4% | 94.5% | 93.9% | 87.0% | 88.7% |
| MED13L | 84.2% | 92.5% | 95.3% | 94.6% | 97.1% | 94.5% | 94.5% |
| CHML | 70.3% | 81.2% | 93.4% | 93.7% | 93.1% | 84.9% | 82.0% |
| TULP4 | 85.3% | 89.9% | 94.5% | 94.9% | 95.8% | 91.0% | 88.2% |
| ZZEF1 | 98.0% | 97.5% | 96.0% | 98.5% | 98.4% | 98.5% | 98.1% |
| ACOT7 | 95.8% | 95.9% | 95.7% | 96.1% | 95.1% | 95.1% | 95.8% |
| LRPAP1 | 94.0% | 96.1% | 94.6% | 92.7% | 91.1% | 91.0% | 85.6% |
| PALM.2 | 12.4% | 45.7% | 92.6% | 94.4% | 87.8% | 58.2% | 63.1% |
| PALM.1 | 14.4% | 46.8% | 90.4% | 92.4% | 87.4% | 58.2% | 63.5% |
| ESRP2 | 19.8% | 77.2% | 48.3% | 84.5% | 71.2% | 75.8% | 35.4% |
| MEF2B | 12.8% | 66.3% | 21.4% | 84.3% | 67.5% | 57.9% | 20.2% |
| REC8 | 17.2% | 64.8% | 26.1% | 69.4% | 47.5% | 39.7% | 13.0% |
| PDYN-AS1 | 39.5% | 80.4% | 78.9% | 84.2% | 43.6% | 62.5% | 28.9% |
| GIMAP7 | 76.6% | 86.1% | 73.9% | 63.3% | 12.5% | 8.1% | 10.1% |
| XXYLT1 | 66.6% | 82.4% | 88.9% | 86.9% | 38.6% | 17.4% | 21.3% |
| HIVEP3 | 55.9% | 81.9% | 58.3% | 64.0% | 24.7% | 37.8% | 17.8% |
| WT1 | 68.5% | 80.8% | 72.9% | 72.1% | 43.7% | 36.8% | 15.1% |
| CD34.2 | 69.4% | 70.6% | 88.6% | 82.3% | 22.7% | 9.0% | 13.5% |
| CD34.1 | 86.6% | 80.7% | 92.0% | 86.8% | 34.8% | 14.8% | 22.6% |
| AIM2 | 10.3% | 13.8% | 57.6% | 67.1% | 89.3% | 81.9% | 78.3% |
| A4GALT | 21.4% | 21.0% | 55.6% | 69.5% | 82.5% | 78.5% | 79.3% |
| CTTN | 14.6% | 11.4% | 55.0% | 68.2% | 85.3% | 60.9% | 80.3% |
| CELF2 | 14.4% | 21.7% | 16.7% | 31.4% | 91.6% | 90.2% | 80.6% |
| HOXB-AS3.1 | 9.9% | 11.5% | 15.8% | 26.2% | 76.5% | 62.3% | 43.9% |
| MIRLET7BHG | 6.4% | 12.7% | 8.6% | 86.2% | 92.2% | 83.9% | 78.8% |
| HOXB3.2.2 | 11.7% | 22.4% | 25.4% | 79.0% | 90.4% | 87.0% | 84.3% |
| HOXB3.2.1 | 14.6% | 26.7% | 33.8% | 86.1% | 92.0% | 92.1% | 89.4% |
| HOXB3.3 | 6.4% | 17.5% | 19.8% | 72.4% | 86.8% | 80.7% | 79.9% |

1. A method of treating a subject with cancer, the method comprising:
a) obtaining a tissue sample from the subject;
b) extracting a nucleic acid from the tissue sample;
c) analyzing an epigenetic pattern of the nucleic acid;
d) comparing the epigenetic pattern from the subject to a control panel;
e) categorizing the subject into an epitype selected from epitype 1, epitype 2, epitype 3, epitype 4, epitype 5, epitype 6, epitype 7, epitype 8, epitype 9, epitype 10, epitype 11, epitype 12, or epitype 13 based on the epigenetic pattern; and
f) administering a treatment to the subject according to the at least one epitype.

2. The method of claim 1, wherein the epigenetic pattern comprises a methylation of a deoxyribonucleic acid (DNA) sequence.

3. The method of claim 2, wherein the methylation comprises a hypermethylation or a hypomethylation.

4. The method of claim 1, wherein the methylation occurs at a cytosine-phosphate-guanosine (CpG) island of the nucleic acid.

5. The method of claim 1, wherein the cancer comprises an acute myeloid leukemia (AML).

6. The method of claim 1, wherein the treatment method comprises regular monitoring by a physician.

7. The method of claim 1, wherein the treatment comprises a drug.

8. The method of claim 7, wherein the drug is a Menin inhibitor.

9. The method of claim 1, wherein the subject retains a methylation pattern associated with a tumor genetic marker yet lacks the tumor genetic marker.

10. The method of claim 9, wherein the genetic marker comprises FLT3-ITD, KMT2A, or NPM1.

11. The method of claim 1, wherein the thirteen epitypes are further divided into 4 superclusters (SC) selected from a transcription factor (TF)-SC, a MLL-SC, a NPM1-SC, or a stem-cell like (SL)-SC.

12. The method of 11, wherein the TF-SC comprises epitype 1, epitype 2, epitype 3, or epitype 4.

13. The method of claim 11, wherein the TF-SC comprises a disruption to one or more transcription factors (TFs).

14. The method of claim 11, wherein the MLL-SC comprises epitype 5 or epitype 6.

15. The method of claim 11, wherein the MLL-SC comprises a rearrangement of a KMT2A/MLL gene.

16. The method of claim 11, wherein the NPM1-SC comprises epitype 7, epitype 8, epitype 9, or epitype 10.

17. The method of claim 11, wherein the NPM1-SC comprises at least one NPM1 mutation.

18. The method of claim 11, wherein the SL-SC comprises epitype 11, epitype 12, or epitype 13.

19. The method of claim 11, wherein the SL-SC displays DNA methylation patterns similar to DNA methylation patterns in hematopoietic stem cells.

20. The method of claim 3, wherein the hypomethylation occurs at a signal transducer and activator of transcription (STAT) gene.

21-32. (canceled)
